SEQUENCE LISTING (113 sequences)

SEQ ID NO: 1 - DNA, 528 bp, Homo sapiens
  CDS (1)..(525)
gttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctga

SEQ ID NO: 2 - protein, 175 aa, Homo sapiens
VPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 3 - DNA, 531 bp, Homo sapiens
  CDS (1)..(528)
atggttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctga

SEQ ID NO: 4 - protein, 176 aa, Homo sapiens
MVPVARLHGALPDARGCHIAQFKSLSPQELQAFKRAKDALEESLLLKDCRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 5 - DNA, 528 bp, Homo sapiens
  CDS (1)..(525)
  misc_feature (146)..(147): n =  A, T, G or C
atggttccggttgctcgtctgcacggtgctctgccggacgctcgtggttgccacatcgctcagttcaaatctctgtctccgcaggaactgcaggctttcaaacgtgctaaagacgctctggaagaatctctgctgctgaaagacdnncgttgccactctcgtctgttcccgcgtacctgggacctgcgtcagctgcaggttcgtgaacgtccgatggctctggaagctgaactggctctgaccctgaaagttctggaagctaccgctgacaccgacccggctctggttgacgttctggaccagccgctgcacaccctgcaccacatcctgtctcagttccgtgcttgcatccagccgcagccgaccgctggtccgcgtacccgtggtcgtctgcaccactggctgtaccgtctgcaggaagctccgaaaaaagaatctccgggttgcctggaagcttctgttaccttcaacctgttccgtctgctgacccgtgacctgaactgcgttgcttctggtgacctgtgcgtt

SEQ ID NO: 6 - protein, 175 aa, Homo sapiens
  misc_feature (49)..(49): The ′Xaa′ at location 49 stands for Lys, Asn, Arg, Ser, Thr, Ile, Met, Glu, Asp, Gly, Ala, Val, Tyr, Trp, Cys, Leu, or Phe.
MVPVARLHGALPDARGCHIAQFKSLSPQELQAFKRAKDALEESLLLKDXRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLC

SEQ ID NO: 7 - DNA, 531 bp, Homo sapiens
  CDS (1)..(528)
atggttcctgtcgccaggctccacggggctctcccggatgcaaggggctgccacatagcccagttcaagtccctgtctccacaggagctgcaggcctttaagagggccaaagatgccttagaagagtcgcttctgctgaaggactccaggtgccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctga

SEQ ID NO: 8 - protein, 176 aa, Homo sapiens
MVPVARLHGALPDARGCHIAQFKSLSPQELQAFKRAKDALEESLLLKDSRCHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 9 - DNA, 528 bp, Homo sapiens
  CDS (1)..(525)
  misc_feature (152)..(153): n = A, T, G or C
atggttccggttgctcgtctgcacggtgctctgccggacgctcgtggttgccacatcgctcagttcaaatctctgtctccgcaggaactgcaggctttcaaacgtgctaaagacgctctggaagaatctctgctgctgaaagactgccgtdnncactctcgtctgttcccgcgtacctgggacctgcgtcagctgcaggttcgtgaacgtccgatggctctggaagctgaactggctctgaccctgaaagttctggaagctaccgctgacaccgacccggctctggttgacgttctggaccagccgctgcacaccctgcaccacatcctgtctcagttccgtgcttgcatccagccgcagccgaccgctggtccgcgtacccgtggtcgtctgcaccactggctgtaccgtctgcaggaagctccgaaaaaagaatctccgggttgcctggaagcttctgttaccttcaacctgttccgtctgctgacccgtgacctgaactgcgttgcttctggtgacctgtgcgtt

SEQ ID NO: 10 - protein, 175 aa, Homo sapiens
  misc_feature (51)..(51): The ′Xaa′ at location 51 stands for Lys, Asn, Arg, Ser, Thr, Ile, Met, Glu, Asp, Gly, Ala, Val, Tyr, Trp, Cys, Leu, or Phe.
MVPVARLHGALPDARGCHIAQFKSLSPQELQAFKRAKDALEESLLLKDCRXHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLC

SEQ ID NO: 11 - DNA, 531 bp, Homo sapiens
  CDS (1)..(528)
atggttcctgtcgccaggctccacggggctctcccggatgcaaggggctgccacatagcccagttcaagtccctgtctccacaggagctgcaggcctttaagagggccaaagatgccttagaagagtcgcttctgctgaaggactgcaggtcccactcccgcctcttccccaggacctgggacctgaggcagctgcaggtgagggagcgccccatggctttggaggctgagctggccctgacgctgaaggttctggaggccaccgctgacactgacccagccctggtggacgtcttggaccagccccttcacaccctgcaccatatcctctcccagttccgggcctgtatccagcctcagcccacggcagggcccaggacccggggccgcctccaccattggctgtaccggctccaggaggccccaaaaaaggagtcccctggctgcctcgaggcctctgtcaccttcaacctcttccgcctcctcacgcgagacctgaattgtgttgccagtggggacctgtgtgtctga

SEQ ID NO: 12 - protein, 176 aa, Homo sapiens
MVPVARLHGALPDARGCHIAQFKSLSPQELQAFKRAKDALEESLLLKDCRSHSRLFPRTWDLRQLQVRERPMALEAELALTLKVLEATADTDPALVDVLDQPLHTLHHILSQFRACIQPQPTAGPRTRGRLHHWLYRLQEAPKKESPGCLEASVTFNLFRLLTRDLNCVASGDLCV

SEQ ID NO: 13 - DNA, 528 bp, Homo sapiens
  CDS (1)..(525)
gttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 14 - protein, 175 aa, Homo sapiens
VPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 15 - DNA, 531 bp, Homo sapiens
  CDS (1)..(528)
atggttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 16 - protein, 176 aa, Homo sapiens
MVPVARLRGALPDARGCHIAQFKSLSPQELQAFKRAKDALEESLLLKDCKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 17 - DNA, 528 bp, Homo sapiens
  CDS (1)..(525)
  misc_feature (146)..(147): n = A, T, G or C
atggttccggttgctcgtctgcgtggtgctctgccggacgctcgtggttgccacatcgctcagttcaaatctctgtctccgcaggaactgcaggctttcaaacgtgctaaagacgctctggaagaatctctgctgctgaaagacdnnaaatgccgttctcgtctgttcccgcgtacctgggacctgcgtcagctgcaggttcgtgaacgtccggttgctctggaagctgaactggctctgaccctgaaagttctggaagctwsngctgacaccgacccggctctgggtgacgttctggaccagccgctgcacaccctgcaccacatcctgtctcagctgcgtgcttgcatccagccgcagccgaccgctggtccgcgtacccgtggtcgtctgcaccactggctgyaycgtctgcaggaagctccgaaaaaagaatctccgggttgcctggaagcttctgttaccttcaacctgttccgtctgctgacccgtgacctgaactgcgttgcttctggtgacctgtgcgtt

SEQ ID NO: 18 - protein, 175 aa, Homo sapiens
  misc_feature (49)..(49): The ′Xaa′ at location 49 stands for Lys, Asn, Arg, Ser, Thr, Ile, Met, Glu, Asp, Gly, Ala, Val, Tyr, Trp, Cys, Leu, or Phe.
MVPVARLRGALPDARGCHIAQFKSLSPQELQAFKRAKDALEESLLLKDXKCRSRLFPRTWDLRQLQVRERPVALEAELALTLKVLEASADTDPALGDVLDQPLHTLHHILSQLRACIQPQPTAGPRTRGRLHHWLXRLQEAPKKESPGCLEASVTFNLFRLLTRDLNCVASGDLC

SEQ ID NO: 19 - DNA, 531 bp, Homo sapiens
  CDS (1)..(528)
  misc_feature (146)..(147): n = A, T, G or C
atggttcctgtcgccaggctccgcggggctctcccggatgcaaggggctgccacatagcccagttcaagtccctgtctccacaggagctgcaggcctttaagagggccaaagatgccttagaagagtcgcttctgctgaaggacdnnaagtgccgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 20 - protein, 176 aa, Homo sapiens
  misc_feature (49)..(49): The ′Xaa′ at location 49 stands for Lys, Asn, Arg, Ser, Thr, Ile, Met, Glu, Asp, Gly, Ala, Val, Tyr, Trp, Cys, Leu, or Phe.
MVPVARLRGALPDARGCHIAQFKSLSPQELQAFKRAKDALEESLLLKDXKCRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 21 - DNA, 528 bp, Homo sapiens
  CDS (1)..(525)
  misc_feature (152)..(153): n = A, T, G or C
atggttccggttgctcgtctgcgtggtgctctgccggacgctcgtggttgccacatcgctcagttcaaatctctgtctccgcaggaactgcaggctttcaaacgtgctaaagacgctctggaagaatctctgctgctgaaagactgcaaadnncgttctcgtctgttcccgcgtacctgggacctgcgtcagctgcaggttcgtgaacgtccggttgctctggaagctgaactggctctgaccctgaaagttctggaagctwsngctgacaccgacccggctctgggtgacgttctggaccagccgctgcacaccctgcaccacatcctgtctcagctgcgtgcttgcatccagccgcagccgaccgctggtccgcgtacccgtggtcgtctgcaccactggctgyaycgtctgcaggaagctccgaaaaaagaatctccgggttgcctggaagcttctgttaccttcaacctgttccgtctgctgacccgtgacctgaactgcgttgcttctggtgacctgtgcgtt

SEQ ID NO: 22 - protein, 175 aa, Homo sapiens
  misc_feature (51)..(51): The ′Xaa′ at location 51 stands for Lys, Asn, Arg, Ser, Thr, Ile, Met, Glu, Asp, Gly, Ala, Val, Tyr, Trp, Cys, Leu, or Phe.
MVPVARLRGALPDARGCHIAQFKSLSPQELQAFKRAKDALEESLLLKDCKXRSRLFPRTWDLRQLQVRERPVALEAELALTLKVLEASADTDPALGDVLDQPLHTLHHILSQLRACIQPQPTAGPRTRGRLHHWLXRLQEAPKKESPGCLEASVTFNLFRLLTRDLNCVASGDLC

SEQ ID NO: 23 - DNA, 531 bp, Homo sapiens
  CDS (1)..(528)
  misc_feature (152)..(153): n = A, T, G or C
atggttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagdnncgctcccgcctcttccccaggacctgggacctgaggcagctgcaggtgagggagcgccccgtggctttggaggctgagctggccctgacgctgaaggttctggaggccaccgctgacactgacccagccctgggggatgtcttggaccagccccttcacaccctgcaccatatcctctcccagctccgggcctgtatccagcctcagcccacggcagggcccaggacccggggccgcctccaccattggctgcaccggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 24 - protein, 176 aa, Homo sapiens
  misc_feature (51)..(51): The ′Xaa′ at location 51 stands for Lys, Asn, Arg, Ser, Thr, Ile, Met, Glu, Asp, Gly, Ala, Val, Tyr, Trp, Cys, Leu, or Phe.
MVPVARLRGALPDARGCHIAQFKSLSPQELQAFKRAKDALEESLLLKDCKXRSRLFPRTWDLRQLQVRERPVALEAELALTLKVLEATADTDPALGDVLDQPLHTLHHILSQLRACIQPQPTAGPRTRGRLHHWLHRLQEAPKKESPGCLEASVTFNLFRLLTRDLNCVASGDLCV

SEQ ID NO: 25 - DNA, 528 bp, Homo sapiens
  CDS (1)..(525)
  misc_feature (143)..(144): n = A, T, G or C
gttcctgtcgccaggctccgcggggctctcccggatgcaaggggctgccacatagcccagttcaagtccctgtctccacaggagctgcaggcctttaagagggccaaagatgccttagaagagtcgcttctgctgaaggacdnnaagtgccgctcccgcctcttccccaggacctgggacctgaggcagctgcaggtgagggagcgccccgtggctttggaggctgagctggccctgacgctgaaggttctggaggccwsngctgacactgacccagccctgggggatgtcttggaccagccccttcacaccctgcaccatatcctctcccagctccgggcctgtatccagcctcagcccacggcagggcccaggacccggggccgcctccaccattggctgtaycggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 26 - protein, 175 aa, Homo sapiens
  misc_feature (48)..(48): The ′Xaa′ at location 48 stands for Lys, Asn, Arg, Ser, Thr, Ile, Met, Glu, Asp, Gly, Ala, Val, Tyr, Trp, Cys, Leu, or Phe.
VPVARLRGALPDARGCHIAQFKSLSPQELQAFKRAKDALEESLLLKDXKCRSRLFPRTWDLRQLQVRERPVALEAELALTLKVLEASADTDPALGDVLDQPLHTLHHILSQLRACIQPQPTAGPRTRGRLHHWLYRLQEAPKKESPGCLEASVTFNLFRLLTRDLNCVASGDLCV

SEQ ID NO: 27 - DNA, 531 bp, Homo sapiens
  CDS (1)..(528)
  misc_feature (1)..(528): n = A, T, G or C
atggttcctgtcgccaggctccgcggggctctcccggatgcaaggggctgccacatagcccagttcaagtccctgtctccacaggagctgcaggcctttaagagggccaaagatgccttagaagagtcgcttctgctgaaggacdnnaagtgccgctcccgcctcttccccaggacctgggacctgaggcagctgcaggtgagggagcgccccgtggctttggaggctgagctggccctgacgctgaaggttctggaggccwsngctgacactgacccagccctgggggatgtcttggaccagccccttcacaccctgcaccatatcctctcccagctccgggcctgtatccagcctcagcccacggcagggcccaggacccggggccgcctccaccattggctgtaycggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 28 - protein, 176 aa, Homo sapiens
  misc_feature (49)..(49): The ′Xaa′ at location 49 stands for Lys, Asn, Arg, Ser, Thr, Ile, Met, Glu, Asp, Gly, Ala, Val, Tyr, Trp, Cys, Leu, or Phe.
MVPVARLRGALPDARGCHIAQFKSLSPQELQAFKRAKDALEESLLLKDXKCRSRLFPRTWDLRQLQVRERPVALEAELALTLKVLEASADTDPALGDVLDQPLHTLHHILSQLRACIQPQPTAGPRTRGRLHHWLYRLQEAPKKESPGCLEASVTFNLFRLLTRDLNCVASGDLCV

SEQ ID NO: 29 - DNA, 528 bp, Homo sapiens
  CDS (1)..(525)
  misc_feature (149)..(150): n = A, T, G or C
gttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagdnncgctcccgcctcttccccaggacctgggacctgaggcagctgcaggtgagggagcgccccgtggctttggaggctgagctggccctgacgctgaaggttctggaggccwsngctgacactgacccagccctgggggatgtcttggaccagccccttcacaccctgcaccatatcctctcccagctccgggcctgtatccagcctcagcccacggcagggcccaggacccggggccgcctccaccattggctgtaycggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 30 - protein, 175 aa, Homo sapiens
  misc_feature (50)..(50): The ′Xaa′ at location 50 stands for Lys, Asn, Arg, Ser, Thr, Ile, Met, Glu, Asp, Gly, Ala, Val, Tyr, Trp, Cys, Leu, or Phe.
VPVARLRGALPDARGCHIAQFKSLSPQELQAFKRAKDALEESLLLKDCKXRSRLFPRTWDLRQLQVRERPVALEAELALTLKVLEASADTDPALGDVLDQPLHTLHHILSQLRACIQPQPTAGPRTRGRLHHWLYRLQEAPKKESPGCLEASVTFNLFRLLTRDLNCVASGDLCV

SEQ ID NO: 31 - DNA, 531 bp, Homo sapiens
  CDS (1)..(528)
  misc_feature (152)..(153): n =  A, T, G or C
atggttcctgtcgccaggctccgcggggctctcccggatgcaaggggctgccacatagcccagttcaagtccctgtctccacaggagctgcaggcctttaagagggccaaagatgccttagaagagtcgcttctgctgaaggactgcaagdnncgctcccgcctcttccccaggacctgggacctgaggcagctgcaggtgagggagcgccccgtggctttggaggctgagctggccctgacgctgaaggttctggaggccwsngctgacactgacccagccctgggggatgtcttggaccagccccttcacaccctgcaccatatcctctcccagctccgggcctgtatccagcctcagcccacggcagggcccaggacccggggccgcctccaccattggctgtaycggctccaggaggccccaaaaaaggagtcccctggctgcctcgaggcctctgtcaccttcaacctcttccgcctcctcacgcgagacctgaattgtgttgccagcggggacctgtgtgtctga

SEQ ID NO: 32 - protein, 176 aa, Homo sapiens
  misc_feature (51)..(51): The ′Xaa′ at location 51 stands for Lys, Asn, Arg, Ser, Thr, Ile, Met, Glu, Asp, Gly, Ala, Val, Tyr, Trp, Cys, Leu, or Phe.
MVPVARLRGALPDARGCHIAQFKSLSPQELQAFKRAKDALEESLLLKDCKXRSRLFPRTWDLRQLQVRERPVALEAELALTLKVLEASADTDPALGDVLDQPLHTLHHILSQLRACIQPQPTAGPRTRGRLHHWLYRLQEAPKKESPGCLEASVTFNLFRLLTRDLNCVASGDLCV

SEQ ID NO: 33 - DNA, 546 bp, Homo sapiens
  CDS (1)..(543)
ggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgtgtctgagaacgtcaacccaccctgagtccacctga

SEQ ID NO: 34 - protein, 181 aa, Homo sapiens
GPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 35 - DNA, 549 bp, Homo sapiens
  CDS (1)..(546)
  misc_feature (33)..(33): n =  A, T, G or C
atgggtccggttccgacctctaaaccaaccmcnactggtaaaggtdnncacatcgrncgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtrayctgtgcctgcgtacctctacccatccggaatctacctaa

SEQ ID NO: 36 - protein, 182 aa, Homo sapiens
  misc_feature (11)..(11): The ′Xaa′ at location 11 stands for Thr, or Pro.
MGPVPTSKPTXTGKGXHIXRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGBLCLRTSTHPEST

SEQ ID NO: 37 - DNA, 549 bp, Homo sapiens
  CDS (1)..(546)
  misc_feature (33)..(33): n =  A, T, G or C
atgggtccggttccgacctctaaaccaaccmcnactggtaaaggttgccacatcgrncgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtrayctgdnnctgcgtacctctacccatccggaatctacctaa

SEQ ID NO: 38 - protein, 182 aa, Homo sapiens
  misc_feature (11)..(11): The ′Xaa′ at location 11 stands for Thr, or Pro.
MGPVPTSKPTXTGKGCHIXRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGBLXLRTSTHPEST

SEQ ID NO: 39 - DNA, 549 bp, Homo sapiens
  CDS (1)..(546)
atgggtccggttccgacctctaaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtaacctgtctctgcgtacctctacccatccggaatctacctaa

SEQ ID NO: 40 - protein, 182 aa, Homo sapiens
MGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLSLRTSTHPEST

SEQ ID NO: 41 - DNA, 546 bp, Homo sapiens
  CDS (1)..(543)
  misc_feature (44)..(45): n =  A, T, G or C
ggccctgtccccacttccaagcccaccacaactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 42 - protein, 181 aa, Homo sapiens
  misc_feature (15)..(15): The ′Xaa′ at location 15 stands for Lys, Asn, Arg, Ser, Thr, Ile, Met, Glu, Asp, Gly, Ala, Val, Tyr, Trp, Cys, Leu, or Phe.
GPVPTSKPTTTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 43 - DNA, 549 bp, Homo sapiens
  CDS (1)..(546)
  misc_feature (47)..(48): n =  A, T, G or C
atgggccctgtccccacttccaagcccaccacaactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 44 - protein, 182 aa, Homo sapiens
  misc_feature (16)..(16): The ′Xaa′ at location 16 stands for Lys, Asn, Arg, Ser, Thr, Ile, Met, Glu, Asp, Gly, Ala, Val, Tyr, Trp, Cys, Leu, or Phe.
MGPVPTSKPTTTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 45 - DNA, 546 bp, Homo sapiens
  CDS (1)..(543)
  misc_feature (44)..(45): n =  A, T, G or C
ggccctgtccccacttccaagcccaccacaactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 46 - protein, 181 aa, Homo sapiens
  misc_feature (15)..(15): The ′Xaa′ at location 15 stands for Lys, Asn, Arg, Ser, Thr, Ile, Met, Glu, Asp, Gly, Ala, Val, Tyr, Trp, Cys, Leu, or Phe.
GPVPTSKPTTTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 47 - DNA, 549 bp, Homo sapiens
  CDS (1)..(546)
  misc_feature (47)..(48): n =  A, T, G or C
atgggccctgtccccacttccaagcccaccacaactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 48 - protein, 182 aa, Homo sapiens
  misc_feature (16)..(16): The ′Xaa′ at location 16 stands for Lys, Asn, Arg, Ser, Thr, Ile, Met, Glu, Asp, Gly, Ala, Val, Tyr, Trp, Cys, Leu, or Phe.
MGPVPTSKPTTTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 49 - DNA, 546 bp, Homo sapiens
  CDS (1)..(543)
  misc_feature (512)..(513): n =  A, T, G or C
ggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 50 - protein, 181 aa, Homo sapiens
  misc_feature (171)..(171): The ′Xaa′ at location 171 stands for Lys, Asn, Arg, Ser, Thr, Ile, Met, Glu, Asp, Gly, Ala, Val, Tyr, Trp, Cys, Leu, or Phe.
GPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 51 - DNA, 549 bp, Homo sapiens
  CDS (1)..(546)
  misc_feature (1)..(546): n =  A, T, G or C
atgggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 52 - protein, 182 aa, Homo sapiens
  misc_feature (172)..(172): The ′Xaa′ at location 172 stands for Lys, Asn, Arg, Ser, Thr, Ile, Met, Glu, Asp, Gly, Ala, Val, Tyr, Trp, Cys, Leu, or Phe.
MGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 53 - DNA, 546 bp, Homo sapiens
  CDS (1)..(543)
  misc_feature (30)..(30): n =  A, T, G or C
ggccctgtccccacttccaagcccaccccnactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 54 - protein, 181 aa, Homo sapiens
  misc_feature (171)..(171): The ′Xaa′ at location 171 stands for Lys, Asn, Arg, Ser, Thr, Ile, Met, Glu, Asp, Gly, Ala, Val, Tyr, Trp, Cys, Leu, or Phe.
GPVPTSKPTPTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 55 - DNA, 549 bp, Homo sapiens
  CDS (1)..(546)
  misc_feature (33)..(33): n =  A, T, G or C
atgggccctgtccccacttccaagcccaccccnactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 56 - protein, 182 aa, Homo sapiens
  misc_feature (172)..(172): The ′Xaa′ at location 172 stands for Lys, Asn, Arg, Ser, Thr, Ile, Met, Glu, Asp, Gly, Ala, Val, Tyr, Trp, Cys, Leu, or Phe.
MGPVPTSKPTPTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 57 - DNA, 546 bp, Homo sapiens
  CDS (1)..(543)
  misc_feature (30)..(30): n =  A, T, G or C
ggccctgtccccacttccaagcccaccccnactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 58 - protein, 181 aa, Homo sapiens
  misc_feature (15)..(15): The ′Xaa′ at location 15 stands for Lys, Asn, Arg, Ser, Thr, Ile, Met, Glu, Asp, Gly, Ala, Val, Tyr, Trp, Cys, Leu, or Phe.
GPVPTSKPTPTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 59 - DNA, 549 bp, Homo sapiens
  CDS (1)..(546)
  misc_feature (33)..(33): n =  A, T, G or C
atgggccctgtccccacttccaagcccaccccnactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 60 - protein, 182 aa, Homo sapiens
  misc_feature (16)..(16): The ′Xaa′ at location 16 stands for Lys, Asn, Arg, Ser, Thr, Ile, Met, Glu, Asp, Gly, Ala, Val, Tyr, Trp, Cys, Leu, or Phe.
MGPVPTSKPTPTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 61 - DNA, 546 bp, Homo sapiens
  CDS (1)..(543)
  misc_feature (30)..(30): n =  A, T, G or C
ggccctgtccccacttccaagcccaccccnactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 62 - protein, 181 aa, Homo sapiens
  misc_feature (171)..(171): The ′Xaa′ at location 171 stands for Lys, Asn, Arg, Ser, Thr, Ile, Met, Glu, Asp, Gly, Ala, Val, Tyr, Trp, Cys, Leu, or Phe.
GPVPTSKPTPTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 63 - DNA, 546 bp, Homo sapiens
  CDS (1)..(543)
  misc_feature (33)..(33): n =  A, T, G or C
atgggccctgtccccacttccaagcccaccccnactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacc

SEQ ID NO: 64 - protein, 181 aa, Homo sapiens
  misc_feature (172)..(172): The ′Xaa′ at location 172 stands for Lys, Asn, Arg, Ser, Thr, Ile, Met, Glu, Asp, Gly, Ala, Val, Tyr, Trp, Cys, Leu, or Phe.
MGPVPTSKPTPTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPES

SEQ ID NO: 65 - DNA, 546 bp, Homo sapiens
  CDS (1)..(543)
  misc_feature (30)..(30): n =  A, T, G or C
ggccctgtccccacttccaagcccaccccnactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 66 - protein, 181 aa, Homo sapiens
  misc_feature (15)..(15): The ′Xaa′ at location 15 stands for Lys, Asn, Arg, Ser, Thr, Ile, Met, Glu, Asp, Gly, Ala, Val, Tyr, Trp, Cys, Leu, or Phe.
GPVPTSKPTPTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 67 - DNA, 549 bp, Homo sapiens
  CDS (1)..(546)
  misc_feature (33)..(33): n =  A, T, G or C
atgggccctgtccccacttccaagcccaccccnactgggaagggcdnncacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 68 - protein, 182 aa, Homo sapiens
  misc_feature (16)..(16): The ′Xaa′ at location 16 stands for Lys, Asn, Arg, Ser, Thr, Ile, Met, Glu, Asp, Gly, Ala, Val, Tyr, Trp, Cys, Leu, or Phe.
MGPVPTSKPTPTGKGXHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 69 - DNA, 546 bp, Homo sapiens
  CDS (1)..(543)
  misc_feature (512)..(513): n =  A, T, G or C
ggccctgtccccacttccaagcccaccacaactgggaagggctgccacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 70 - protein, 181 aa, Homo sapiens
  misc_feature (171)..(171): The ′Xaa′ at location 171 stands for Lys, Asn, Arg, Ser, Thr, Ile, Met, Glu, Asp, Gly, Ala, Val, Tyr, Trp, Cys, Leu, or Phe.
GPVPTSKPTTTGKGCHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 71 - DNA, 549 bp, Homo sapiens
  CDS (1)..(546)
  misc_feature (515)..(516): n =  A, T, G or C
atgggccctgtccccacttccaagcccaccacaactgggaagggctgccacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 72 - protein, 182 aa, Homo sapiens
  misc_feature (172)..(172): The ′Xaa′ at location 172 stands for Lys, Asn, Arg, Ser, Thr, Ile, Met, Glu, Asp, Gly, Ala, Val, Tyr, Trp, Cys, Leu, or Phe.
MGPVPTSKPTTTGKGCHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 73 - DNA, 546 bp, Homo sapiens
  CDS (1)..(543)
  misc_feature (44)..(45): n =  A, T, G or C
ggccctgtccccacttccaagcccaccacaactgggaagggcdnncacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 74 - protein, 181 aa, Homo sapiens
  misc_feature (15)..(15): The ′Xaa′ at location 15 stands for Lys, Asn, Arg, Ser, Thr, Ile, Met, Glu, Asp, Gly, Ala, Val, Tyr, Trp, Cys, Leu, or Phe.
GPVPTSKPTTTGKGXHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 75 - DNA, 549 bp, Homo sapiens
  CDS (1)..(546)
  misc_feature (47)..(48): n =  A, T, G or C
atgggccctgtccccacttccaagcccaccacaactgggaagggcdnncacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaayctgtgtctgagaacgtcaacccaccctgagtccacctga

SEQ ID NO: 76 - protein, 182 aa, Homo sapiens
  misc_feature (16)..(16): The ′Xaa′ at location 16 stands for Lys, Asn, Arg, Ser, Thr, Ile, Met, Glu, Asp, Gly, Ala, Val, Tyr, Trp, Cys, Leu, or Phe.
MGPVPTSKPTTTGKGXHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLCLRTSTHPEST

SEQ ID NO: 77 - DNA, 546 bp, Homo sapiens
  CDS (1)..(543)
  misc_feature (512)..(513): n =  A, T, G or C
ggccctgtccccacttccaagcccaccacaactgggaagggctgccacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 78 - protein, 181 aa, Homo sapiens
  misc_feature (171)..(171): The ′Xaa′ at location 171 stands for Lys, Asn, Arg, Ser, Thr, Ile, Met, Glu, Asp, Gly, Ala, Val, Tyr, Trp, Cys, Leu, or Phe.
GPVPTSKPTTTGKGCHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 79 - DNA, 549 bp, Homo sapiens
  CDS (1)..(546)
  misc_feature (515)..(516): n =  A, T, G or C
atgggccctgtccccacttccaagcccaccacaactgggaagggctgccacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgdnnctgagaacgtcaacccaccctgagtccacctga

SEQ ID NO: 80 - protein, 182 aa, Homo sapiens
  misc_feature (172)..(172): The ′Xaa′ at location 172 stands for Lys, Asn, Arg, Ser, Thr, Ile, Met, Glu, Asp, Gly, Ala, Val, Tyr, Trp, Cys, Leu, or Phe.
MGPVPTSKPTTTGKGCHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLXLRTSTHPEST

SEQ ID NO: 81 - DNA, 546 bp, Homo sapiens
  CDS (1)..(543)
  misc_feature (44)..(45): n =  A, T, G or C
ggccctgtccccacttccaagcccaccacaactgggaagggcdnncacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 82 - protein, 181 aa, Homo sapiens
  misc_feature (15)..(15): The ′Xaa′ at location 15 stands for Lys, Asn, Arg, Ser, Thr, Ile, Met, Glu, Asp, Gly, Ala, Val, Tyr, Trp, Cys, Leu, or Phe.
GPVPTSKPTTTGKGXHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 83 - DNA, 549 bp, Homo sapiens
  CDS (1)..(546)
  misc_feature (47)..(48): n =  A, T, G or C
atgggccctgtccccacttccaagcccaccacaactgggaagggcdnncacattgayaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatggggayctgtgtctgagaacgtcaacccaccctgagtccacctga

SEQ ID NO: 84 - protein, 182 aa, Homo sapiens
  misc_feature (16)..(16): The ′Xaa′ at location 16 stands for Lys, Asn, Arg, Ser, Thr, Ile, Met, Glu, Asp, Gly, Ala, Val, Tyr, Trp, Cys, Leu, or Phe.
MGPVPTSKPTTTGKGXHIDRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGDLCLRTSTHPEST

SEQ ID NO: 85 - DNA, 543 bp, Homo sapiens
  CDS (1)..(540)
  misc_feature (509)..(510): n =  A, T, G or C
cctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 86 - protein, 180 aa, Homo sapiens
  misc_feature (170)..(170): The ′Xaa′ at location 170 stands for Lys, Asn, Arg, Ser, Thr, Ile, Met, Glu, Asp, Gly, Ala, Val, Tyr, Trp, Cys, Leu, or Phe.
PVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 87 - DNA, 540 bp, Homo sapiens
  CDS (1)..(537)
  misc_feature (506)..(507): n =  A, T, G or C
gtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 88 - protein, 179 aa, Homo sapiens
  misc_feature (169)..(169): The ′Xaa′ at location 169 stands for Lys, Asn, Arg, Ser, Thr, Ile, Met, Glu, Asp, Gly, Ala, Val, Tyr, Trp, Cys, Leu, or Phe.
VPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 89 - DNA, 537 bp, Homo sapiens
  CDS (1)..(534)
  misc_feature (503)..(504): n =  A, T, G or C
cccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 90 - protein, 178 aa, Homo sapiens
  misc_feature (168)..(168): The ′Xaa′ at location 168 stands for Lys, Asn, Arg, Ser, Thr, Ile, Met, Glu, Asp, Gly, Ala, Val, Tyr, Trp, Cys, Leu, or Phe.
PTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 91 - DNA, 534 bp, Homo sapiens
  CDS (1)..(531)
  misc_feature (500)..(501): n =  A, T, G or C
acttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 92 - protein, 177 aa, Homo sapiens
  misc_feature (167)..(167): The ′Xaa′ at location 167 stands for Lys, Asn, Arg, Ser, Thr, Ile, Met, Glu, Asp, Gly, Ala, Val, Tyr, Trp, Cys, Leu, or Phe.
TSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 93 - DNA, 531 bp, Homo sapiens
  CDS (1)..(528)
  misc_feature (497)..(498): n =  A, T, G or C
tccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 94 - protein, 176 aa, Homo sapiens
  misc_feature (166)..(166): The ′Xaa′ at location 166 stands for Lys, Asn, Arg, Ser, Thr, Ile, Met, Glu, Asp, Gly, Ala, Val, Tyr, Trp, Cys, Leu, or Phe.
SKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 95 - DNA, 528 bp, Homo sapiens
  CDS (1)..(525)
  misc_feature (494)..(495): n =  A, T, G or C
aagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 96 - protein, 175 aa, Homo sapiens
  misc_feature (165)..(165): The ′Xaa′ at location 165 stands for Lys, Asn, Arg, Ser, Thr, Ile, Met, Glu, Asp, Gly, Ala, Val, Tyr, Trp, Cys, Leu, or Phe.
KPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 97 - DNA, 552 bp, Homo sapiens
  CDS (1)..(549)
  misc_feature (6)..(6): n =  A, T, G or C
atgytnggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 98 - protein, 183 aa, Homo sapiens
  misc_feature (173)..(173): The ′Xaa′ at location 173 stands for Lys, Asn, Arg, Ser, Thr, Ile, Met, Glu, Asp, Gly, Ala, Val, Tyr, Trp, Cys, Leu, or Phe.
MLGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 99 - DNA, 549 bp, Homo sapiens
  CDS (1)..(546)
  misc_feature (6)..(6): n =  A, T, G or C
atgytncctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 100 - protein, 182 aa, Homo sapiens
  misc_feature (172)..(172): The ′Xaa′ at location 172 stands for Lys, Asn, Arg, Ser, Thr, Ile, Met, Glu, Asp, Gly, Ala, Val, Tyr, Trp, Cys, Leu, or Phe.
MLPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 101 - DNA, 552 bp, Homo sapiens
  CDS (1)..(549)
  misc_feature (518)..(519): n =  A, T, G or C
atgathggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 102 - protein, 183 aa, Homo sapiens
  misc_feature (173)..(173): The ′Xaa′ at location 173 stands for Lys, Asn, Arg, Ser, Thr, Ile, Met, Glu, Asp, Gly, Ala, Val, Tyr, Trp, Cys, Leu, or Phe.
MIGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 103 - DNA, 549 bp, Homo sapiens
  CDS (1)..(546)
  misc_feature (515)..(516): n =  A, T, G or C
atgathcctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 104 - protein, 182 aa, Homo sapiens
  misc_feature (172)..(172): The ′Xaa′ at location 172 stands for Lys, Asn, Arg, Ser, Thr, Ile, Met, Glu, Asp, Gly, Ala, Val, Tyr, Trp, Cys, Leu, or Phe.
MIPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 105 - DNA, 531 bp, Homo sapiens
  CDS (1)..(531)
atgaaaccaaccaccactggtaaaggttgccacatcggtcgtttcaaatctctgtctccgcaggaactggcttctttcaaaaaagctcgtgacgctctggaagaatctctgaaactgaaaaactggtcttgctcttctccggttttcccgggtaactgggatctgcgtctgctgcaggttcgtgaacgtccggttgctctggaagctgaactggctctgaccctgaaagttctggaagctgctgcaggtcctgctctggaagatgttctggatcagccgctgcacactctgcaccacatcctgtctcagctgcaggcttgcattcaaccgcaaccgaccgctggtccgcgtccgcgtggtcgtctgcaccactggctgcatcgtctgcaggaagctccgaaaaaagaatctgctggttgcctggaagcttctgttaccttcaacctgttccgtctgctgacccgtgatctgaaatacgttgctgatggtaacctgtctctgcgtacctctacccatccggaatctacctaa

SEQ ID NO: 106 - protein, 176 aa, Homo sapiens
MKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLSLRTSTHPEST

SEQ ID NO: 107 - DNA, 531 bp, Homo sapiens
  CDS (1)..(528)
  misc_feature (497)..(498): n =  A, T, G or C
atgaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 108 - protein, 176 aa, Homo sapiens
  misc_feature (166)..(166): The ′Xaa′ at location 166 stands for Lys, Asn, Arg, Ser, Thr, Ile, Met, Glu, Asp, Gly, Ala, Val, Tyr, Trp, Cys, Leu, or Phe.
MKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 109 - DNA, 558 bp, Homo sapiens
  CDS (1)..(555)
  misc_feature (9)..(9): n =  A, T, G or C
atggargcngarggccctgtccccacttccaagcccaccacaactgggaagggctgccacattggcaggttcaaatctctgtcaccacaggagctagcgagcttcaagaaggccagggacgccttggaagagtcactcaagctgaaaaactggagttgcagctctcctgtcttccccgggaattgggacctgaggcttctccaggtgagggagcgccctgtggccttggaggctgagctggccctgacgctgaaggtcctggaggccgctgctggcccagccctggaggacgtcctagaccagccccttcacaccctgcaccacatcctctcccagctccaggcctgtatccagcctcagcccacagcagggcccaggccccggggccgcctccaccactggctgcaccggctccaggaggcccccaaaaaggagtccgctggctgcctggaggcatctgtcaccttcaacctcttccgcctcctcacgcgagacctcaaatatgtggccgatgggaacctgdnnctgagaacgtcaacccaccctgagtccacctga

SEQ ID NO: 110 - protein, 185 aa, Homo sapiens
  misc_feature (175)..(175): The ′Xaa′ at location 175 stands for Lys, Asn, Arg, Ser, Thr, Ile, Met, Glu, Asp, Gly, Ala, Val, Tyr, Trp, Cys, Leu, or Phe.
MEAEGPVPTSKPTTTGKGCHIGRFKSLSPQELASFKKARDALEESLKLKNWSCSSPVFPGNWDLRLLQVRERPVALEAELALTLKVLEAAAGPALEDVLDQPLHTLHHILSQLQACIQPQPTAGPRPRGRLHHWLHRLQEAPKKESAGCLEASVTFNLFRLLTRDLKYVADGNLXLRTSTHPEST

SEQ ID NO: 111 - protein, 520 aa, Homo sapiens
MAGPERWGPLLLCLLQAAPGRPRLAPPQNVTLLSQNFSVYLTWLPGLGNPQDVTYFVAYQSSPTRRRWREVEECAGTKELLCSMMCLKKQDLYNKFKGRVRTVSPSSKSPWVESEYLDYLFEVEPAPPVLVLTQTEEILSANATYQLPPCMPPLDLKYEVAFWKEGAGNKTLFPVTPHGQPVQITLQPAASEHHCLSARTIYTFSVPKYSKFSKPTCFLLEVPEANWAFLVLPSLLILLLVIAAGGVIWKTLMGNPWFQRAKMPRALDFSGHTHPVATFQPSRPESVNDLFLCPQKELTRGVRPTPRVRAPATQQTRWKKDLAEDEEEEDEEDTEDGVSFQPYIEPPSFLGQEHQAPGHSEAGGVDSGRPRAPLVPSEGSSAWDSSDRSWASTVDSSWDRAGSSGYLAEKGPGQGPGGDGHQESLPPPEFSKDSGFLEELPEDNLSSWATWGTLPPEPNLVPGGPPVSLQTLTFCWESSPEEEEEARESEIEDSDAGSWGAESTQRTEDRGRTLGHYMAR

SEQ ID NO: 112 - DNA, 978 bp, Homo sapiens
  CDS (1)..(975)
atggcgtggagccttgggagctggctgggtggctgcctgctggtgtcagcattgggaatggtaccacctcccgaaaatgtcagaatgaattctgttaatttcaagaacattctacagtgggagtcacctgcttttgccaaagggaacctgactttcacagctcagtacctaagttataggatattccaagataaatgcatgaatactaccttgacggaatgtgatttctcaagtctttccaagtatggtgaccacaccttgagagtcagggctgaatttgcagatgagcattcagactgggtaaacatcaccttctgtcctgtggatgacaccattattggaccccctggaatgcaagtagaagtacttgctgattctttacatatgcgtttcttagcccctaaaattgagaatgaatacgaaacttggactatgaagaatgtgtataactcatggacttataatgtgcaatactggaaaaacggtactgatgaaaagtttcaaattactccccagtatgactttgaggtcctcagaaacctggagccatggacaacttattgtgttcaagttcgagggtttcttcctgatcggaacaaagctggggaatggagtgagcctgtctgtgagcaaacaacccatgacgaaacggtcccctcctggatggtggccgtcatcctcatggcctcggtcttcatggtctgcctggcactcctcggctgcttctccttgctgtggtgcgtttacaagaagacaaagtacgccttctcccctaggaattctcttccacagcacctgaaagagtttttgggccatcctcatcataacacacttctgtttttctcctttccattgtcggatgagaatgatgtttttgacaagctaagtgtcattgcagaagactctgagagcggcaagcagaatcctggtgacagctgcagcctcgggaccccgcctgggcaggggccccaaagctag

SEQ ID NO: 113 - protein, 325 aa, Homo sapiens [sequence omitted]

(19) United States
(12) Patent Application Publication
(10) Pub. No.: US 2011/0165121 A1
(43) Pub. Date: Jul. 7, 2011

(54) USE OF PEGYLATED TYPE III INTERFERONS FOR THE TREATMENT OF HEPATITIS C

(76) Inventors: Diana F. Hausman, Seattle, WA (US); Michael G. Dodds, Normandy Park, WA (US)

(21) Appl. No.: 12/996,358

(22) PCT Filed: Jun. 5, 2009
(86) PCT No.: PCT/US09/46451

§ 371 Date: Mar. 23, 2011

Related U.S. Application Data
(60) Provisional application No. 61/059,237, filed on Jun. 5, 2008.
(60) Provisional application No. 61/109,455, filed on Oct. 29, 2008.
(60) Provisional application No. 61/167,763, filed on Apr. 8, 2009.

(51) Int. Cl. A61K 38/21; A61P 31/14; A61P 29/00; A61P 37/02; A61P 37/04; A61P 31/00; A61P 31/12

(52) U.S. Cl. 424/85.5; 424/85.4; 424/85.7

(57) ABSTRACT

Methods for treating human patients infected with the hepatitis C virus using pegylated Type III Interferons (IL-28A, IL-28B and IL-29) alone or in combination with other antiviral agents.

### BACKGROUND OF THE INVENTION

It has been estimated that 3% of the world's population, i.e., 130 million individuals are infected with hepatitis C. Stauber R E and Stadlbauer V., Journal of Clinical Virology, 36:87-94 (2006). The majority have been infected via parenteral exposure with contaminated injections, either related to injection drug use or contaminated injections or transfusion with blood products received as part of an individual' health care. The current standard of care for hepatitis C is pegylated interferon (PEG-IFN) alpha (given once weekly) in combination with oral ribavirin (given daily). Heathcote J. and Main J., Journal of Viral Hepatitis, 12:223-235 (2005).

Chronic infection with hepatitis C virus (HCV) is a leading cause of cirrhosis, liver failure, and hepatocellular carcinoma in the United States and worldwide. The primary goal of treatment is to eradicate the virus and prevent development of long-term complications. Successful treatment is defined as achievement of a sustained virologic response (SVR) as evidenced by undetectable HCV RNA levels at least 6 months following discontinuation of therapy (Pearlman B L. Hepatitis C treatment update. Am J Med 2004; 117(5):344-352).

For patients infected with genotype 1 HCV, the most common genotype in the United States, treatment consists of weekly administration of a PEGylated interferon alpha (PEG-IFN-α) in combination with daily ribavirin for 48 weeks. The two currently approved forms of PEG-IFN-α are peginterferon alpha-2a (PEGASYS®), and peginterferon alpha-2b (PEG-INTRON®), both of which are associated with SVR rates of about 50% in patients infected with genotype 1 HCV (Seeff L B. Natural history of chronic hepatitis C. Hepatology 2002A; 36(5 Suppl 1):535-46; Strader D B, Wright T, Thomas D L, Seeff L B. Diagnosis, management, and treatment of hepatitis C. Hepatology 2004; 39(4):1147-1171). For those patients who fail to achieve an SVR, there is currently no standard treatment.

Relapsed patients, who compose about 20% of all treated genotype 1 HCV patients, represent a unique population of PEG-IFN-α treatment failures (Hadziyannis S J, Sette H, Jr., Morgan T R, Balan V, Diago M, Marcellin P, Ramadori G, Bodenheimer H, Jr., Bernstein D, Rizzetto M, Zeuzem S, Pockros P J, Lin A, Ackrill A M. Peginterferon-alpha2a and ribavirin combination therapy in chronic hepatitis C: a randomized study of treatment duration and ribavirin dose. Ann Intern Med 2004; 140(5):346-355). While these patients have undetectable HCV RNA levels at the end of treatment, they relapse with detectable HCV RNA levels less than 6 months later (Hoofnagle J H, Seeff L B. Peginterferon and ribavirin for chronic hepatitis C. N Engl J Med 2006; 355(23):2444-2451). Factors contributing to relapse may include dose reduction in ribavirin, especially during the first 24 weeks of treatment (Shiffman M L. Chronic hepatitis C: treatment of pegylated interferon/ribavirin nonresponders. Curr Gastroenterol Rep 2006; 8(1):46-52.). Upon retreatment with IFN-α-based therapy, relapsed patients may manifest decreases in HCV RNA levels similar to those seen during their prior course of therapy (Strader D B, Wright T, Thomas D L, Seeff L B. Diagnosis, management, and treatment of hepatitis C. Hepatology 2004; 39(4):1147-1171), and in cases where prior therapy consisted of a non-PEGylated IFN-α, may be able to achieve an SVR with retreatment utilizing a PEG-IFN-α and ribavirin (Jacobson I M, et al., A randomized trial of pegylated interferon alpha-2b plus ribavirin in the retreatment of chronic hepatitis C. Am J Gastroenterol 2005; 100(11):2453-2462; Mathew A, et al., Sustained viral response to pegylated interferon alpha-2b and ribavirin in chronic hepatitis C refractory to prior treatment. Dig Dis Sci 2006; 51(11):1956-1961; Shiffman M L., Chronic hepatitis C: treatment of pegylated interferon/ribavirin nonresponders. Curr Gastroenterol Rep 2006; 8(1):46-52). This pattern of failure and response to retreatment suggests that relapsed patients retain the potential to respond to interferon-based therapy and therefore are a unique population in which to study the potential effects of novel interferon-like molecules (Hoofnagle J H, Seeff L B. Peginterferon and ribavirin for chronic hepatitis C. N Engl J Med 2006; 355(23):2444-2451; FDA CDER Antiviral Drugs Advisory Committee. Summary Minutes of the Antiviral Drugs Advisory Committee, Oct. 19-20, 2006).

Treatment with PEG-IFN-α and ribavirin is associated with significant side effects. Major toxicities of PEG-IFN-α include flu-like symptoms; hematologic abnormalities including neutropenia, thrombocytopenia, and anemia; and neuropsychiatric disorders, most commonly depression. Other toxicities include gastrointestinal disturbances and dermatologic, autoimmune, and cardiac conditions. Elevations in liver transaminases have also been reported, particularly with peginterferon alpha 2a (Gish R G. Treating hepatitis C: the state of the art. Gastroenterol Clin North Am 2004; 33(1 Suppl):S1-9; Hoffmann-La Roche Inc. Package Insert: PEGASYS® (peginterferon alfa-2a). 2005B:1-46). Ribavirin is associated with a number of adverse effects, most notably hemolytic anemia, which in combination with the myelosuppressive effects of IFN-α can be a significant clinical problem (Kowdley K V. Hematologic side effects of interferon and ribavirin therapy. J Clin Gastroenterol 2005; 39(1 Suppl):S3-8; Strader D B, Wright T, Thomas D L, Seeff L B. Diagnosis, management, and treatment of hepatitis C. Hepatology 2004; 39(4):1147-1171).

The toxicities associated with PEG-IFN-α and ribavirin often lead to delays in starting therapy, as well as dose reductions and early discontinuation of treatment (Pearlman B L. Hepatitis C treatment update. Am J Med 2004; 117(5):344-352), all of which decrease the likelihood of achieving SVR. Adherence to therapy (defined as receiving ≧80% of the prescribed PEG IFN-α dose and >80% of the ribavirin dose for the duration of therapy) has been associated with higher SVR rates in genotype 1 HCV patients (McHutchison J G, et al., Adherence to combination therapy enhances sustained response in genotype-1-infected patients with chronic hepatitis C. Gastroenterology 2002; 123(4):1061-1069).

Given the efficacy and toxicity limitations of current therapy, there remains a need for improved treatments for HCV. One approach is to develop novel interferon-like molecules that at least improve the tolerability of treatment, leading to fewer dose reductions and treatment discontinuations, and greater adherence to prescribed therapy, which should then translate into improved efficacy. Use of the Type III Interferons can provide such therapeutic improvements for the treatment of HCV.

### DESCRIPTION OF THE INVENTION

I. Definitions

The terms “amino-terminal” and “carboxyl-terminal” are used herein to denote positions within polypeptides. Where the context allows, these terms are used with reference to a particular sequence or portion of a polypeptide to denote proximity or relative position. For example, a certain sequence positioned carboxyl-terminal to a reference sequence within a polypeptide is located proximal to the carboxyl terminus of the reference sequence, but is not necessarily at the carboxyl terminus of the complete polypeptide.

The term “anti-hepatitis C agent” is a molecule that when administered before, concurrently or after administration of a Type III Interferon (pegylated or nonpegylated) to a human patient (“combination therapy”), that the amount of HCV RNA present in the combination-treated human patient is less than the amount of HCV RNA present in the human patient after receiving treatment with Type III Interferon alone. A Type III Interferon can be administered before, concurrently or after administration of at least one or more of the following anti-hepatitis C agents: a polymerase and/or protease inhibitors, A3AR agonists, Toll-Like Receptor agonists, monoclonal antibodies, Botanicals, anti-phospholipids, immunomodulators, anti-inflammatory drugs, thiazolides, broad spectrum immune stimulators, inflammatory/fibrosis inhibitors, cyclophilin inhibitors, pancaspase inhibitors, HCV immune globulins, antivirals, anti-infectives, RNA inhibitors, glucosidase I inhibitors, IRES inhibitors, bezafibrates, nucleoside analogs, a Type I Interferon or a Type II Interferon. Optionally, the polymerase and/or protease inhibitor can be VCH-916 (Virochem), GS9190 (Gilead), GSK625433 (GlaxcoSmithKline), ITMN-191 (R-7227; InterMune), R7128 (Pharmasset/Roche), VCH-759 (Virochem), R1626 (Roche), TMC435350 (Medivir/Tibotec), SCH503034 (Boceprevir, S chering-Plough), A-831 (Arrow Therapeutics), valopicitabine (NM283, Idenix Pharmaceuticals) or VX950 (Telaprevir, Vertex). Optionally, the A3AR agonist is CF102 (Can-Fite). Optionally, the Toll-Like Receptor agonist is IMO-2125 (Idera Pharmaceuticals), Isatoribine (ANA971, Anadys Pharmaceuticals) or Actilon (CPG10101, Coley Pharmaceutical Group). Optionally, the monoclonal antibody is AB68 (XTL bio). Optionally, the Botanical is PYN17 (Phynova). Optionally, the anti-phospholipid is Bavituximab (formerly Tarvacin; Peregrine). Optionally, the immunomodulator is NOV-205 (Novelos Therapeutics), Oglufanide disodium (Implicit Bioscience) or thymalfasin (thymosin alpha 1; SciClone/Sigma-Tau). Optionally, the anti-inflammatory drug is CTS-1027 (Conatus) or JBK-122 (Jenken Biosciences). Optionally, the thiazolide is Alinia (nitazoxanide; Romark Laboratories). Optionally, the broad spectrum immune stimulator is SCV-07 (SciClone). Optionally, the inflammatory/fibrosis inhibitor is MitoQ (mitoquinone; Antipodean Pharmaceuticals). Optionally, the cyclophilin inhibitor is DEBIO-025 (Debio Pharm Group). Optionally, pancaspase inhibitor is PF-03491390 (formerly IDN-6556; Pfizer Pharmaceuticals). Optionally, the HCV immune globulin is Civacir (Nabi). Optionally, the antiviral is Suvus (Methylene blue, formerly BIVN-104 (Virostat); Bioenvision). Optionally, the anti-infective is Nitazoxanide (Alinia®, Romark Pharmaceuticals). Optionally, the glucosidase I inhibitor is MX-3253 (celgosivir; Migenix). Optionally, the IRES inhibitor is VGX-410C (Mifepristone; VGX Pharmaceuticals). Optionally, the bezafibrate is Hepaconda (Giaconda). Optionally, the nucleoside analog is ribavirin (e.g., Roches's Copegus or Schering-Plough's Rebetol) or viramidine (taribavirin (a ribavirin pro-drug); Valeant Pharmaceuticals). Optionally, the ribavirin or viramidine is administered orally once or twice daily to the patient at a dose of about 800-1200 mg. Optionally, the Type I Interferon is Interferon alpha or pegylated Interferon alpha. Optionally, the Interferon alpha or pegylated Interferon alpha is PEGASYS (pegylated interferon-alpha-2a or peg-IFN-α-2a; Roche), PEG-INTRON (pegylated interferon-alpha-2b or peg-IFN-α-2b; Schering-Plough), Belerofon (Nautilus Biotech), oral interferon alpha (Amarillo Biosciences), BLX-883 (Locteron; Biolex Therapeutics/OctoPlus), Multiferon (Viragen), Albuferon (Human Genome Sciences), Consensus Interferon or (Infergen; Three Rivers Pharma). Optionally, the Type I Interferon is omega interferon (Intarcia Therapeutics). Optionally, the Type II Interferon is Interferon gamma, e.g., Actimmune® by Intermune.

The term “degenerate nucleotide sequence” denotes a sequence of nucleotides that includes one or more degenerate codons (as compared to a reference polynucleotide molecule that encodes a polypeptide). Degenerate codons contain different triplets of nucleotides, but encode the same amino acid residue (i.e., GAU and GAC triplets each encode Asp).

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

A “fixed” dose of a therapeutic agent herein refers to a dose that is administered to a human patient without regard for the weight (WT) or body surface area (BSA) of the patient. The fixed dose is therefore not provided as a μg/kg or mg/kg dose, but rather as an absolute amount of the Type III Interferon, Pegylated Type III Interferon or anti-hepatitis C agent.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems. Such isolated molecules are those that are separated from their natural environment and include cDNA and genomic clones. Isolated DNA molecules of the present invention are free of other genes with which they are ordinarily associated, but may include naturally occurring 5′ and 3′ untranslated regions such as promoters and terminators. The identification of associated regions will be evident to one of ordinary skill in the art (see for example, Dynan and Tijan, Nature 316:774-78, 1985).

An “isolated” polypeptide or protein is a polypeptide or protein that is found in a condition other than its native environment, such as apart from blood and animal tissue. In a preferred form, the isolated polypeptide is substantially free of other polypeptides, particularly other polypeptides of animal origin. It is preferred to provide the polypeptides in a highly purified form, i.e. greater than 95% pure, more preferably greater than 99% pure. When used in this context, the term “isolated” does not exclude the presence of the same polypeptide in alternative physical forms, such as dimers or alternatively glycosylated or derivatized forms.

A “loading” dose herein generally comprises an initial dose of a therapeutic agent, e.g., Type III Interferon, Pegylated Type III Interferon or an anti-hepatitis C agent, administered to a patient, and is followed by one or more maintenance dose(s) thereof. Generally, a single loading dose is administered, but multiple loading doses are contemplated herein. Usually, the amount of loading dose(s) administered exceeds the amount of the maintenance dose(s) administered and/or the loading dose(s) are administered more frequently than the maintenance dose(s), so as to achieve the desired steady-state concentration of the therapeutic agent earlier than can be achieved with the maintenance dose(s).

A “maintenance” dose herein refers to one or more doses of a therapeutic agent, e.g., Type III Interferon, Pegylated Type III Interferon or an anti-hepatitis C agent, administered to the patient over a treatment period. The maintenance doses may be administered at spaced treatment intervals, such as about twice a week, every week, about every 2 weeks, about every 3 weeks, or about every 4 weeks.

The term “operably linked”, when referring to DNA segments, indicates that the segments are arranged so that they function in concert for their intended purposes, e.g., transcription initiates in the promoter and proceeds through the coding segment to the terminator.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules. Sizes of polynucleotides are expressed as base pairs (abbreviated “bp”), nucleotides (“nt”), or kilobases (“kb”). Where the context allows, the latter two terms may describe polynucleotides that are single-stranded or double-stranded. When the term is applied to double-stranded molecules it is used to denote overall length and will be understood to be equivalent to the term “base pairs”. It will be recognized by those skilled in the art that the two strands of a double-stranded polynucleotide may differ slightly in length and that the ends thereof may be staggered as a result of enzymatic cleavage; thus all nucleotides within a double-stranded polynucleotide molecule may not be paired.

A “polypeptide” is a polymer of amino acid residues joined by peptide bonds, whether produced naturally or synthetically. Polypeptides of less than about 10 amino acid residues are commonly referred to as “peptides”.

The phrase “prior treatment” refers to the administration of a prior combination therapy which included a Pegylated Interferon alpha (e.g., peginterferon alpha-2a (PEGASYS®), or peginterferon alpha-2b (PEG-INTRON®)) and a nucleoside analog (e.g., ribavirin or viramidine) to a human patient infected with the hepatitis C virus, wherein said prior combination therapy resulted in viral clearance of the hepatitis C virus, i.e., undetectable hepatitis C virus RNA. After about six (6) months following said prior treatment, the patient is tested to determine whether there has been a hepatitis C viral relapse (i.e., detectable HCV RNA greater than or equal to 100,000 International Units per milliliter). Such patients are in the “responders/relapsers” subpopulation of HCV patients.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

A “protein” is a macromolecule comprising one or more polypeptide chains. A protein may also comprise non-peptidic components, such as carbohydrate groups. Carbohydrates and other non-peptidic substituents may be added to a protein by the cell in which the protein is produced, and will vary with the type of cell. Proteins are defined herein in terms of their amino acid backbone structures; substituents such as carbohydrate groups are generally not specified, but may be present nonetheless.

The term “receptor” denotes a cell-associated protein that binds to a bioactive molecule (i.e., a ligand) and mediates the effect of the ligand on the cell. Membrane-bound receptors are characterized by a multi-peptide structure comprising an extracellular ligand-binding domain and an intracellular effector domain that is typically involved in signal transduction. Binding of ligand to receptor results in a conformational change in the receptor that causes an interaction between the effector domain and other molecule(s) in the cell. This interaction in turn leads to an alteration in the metabolism of the cell. Metabolic events that are linked to receptor-ligand interactions include gene transcription, phosphorylation, dephosphorylation, increases in cyclic AMP production, mobilization of cellular calcium, mobilization of membrane lipids, cell adhesion, hydrolysis of inositol lipids and hydrolysis of phospholipids. In general, receptors can be membrane bound, cytosolic or nuclear; monomeric (e.g., thyroid stimulating hormone receptor, beta-adrenergic receptor) or multimeric (e.g., PDGF receptor, growth hormone receptor, IL-3 receptor, GM-CSF receptor, G-CSF receptor, erythropoietin receptor and IL-6 receptor).

The term “secretory signal sequence” denotes a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

“Treatment” or “treating” refers to both therapeutic treatment and prophylactic or preventative measures. Those in need of treatment include those already infected with the hepatitis C virus as well as those in which hepatitis C disease is to be prevented. Hence, the patient to be treated herein may have been diagnosed as having hepatitis C or may be predisposed or susceptible to the disease.

“zcyto20” is a previous designation for “IL-28A” and IL-28A is a previous designation for “Interferon Lambda-2” (IFN-λ2). See, for example, U.S. Pat. Nos. 7,038,032, 6,927,040, 7,135,170, 7,157,559, 7,351,689 and WIPO publication Nos. WO 05/097165, WO 07/012,033, WO 07/013,944 and WO 07/041,713, all of which are herein incorporated by reference in their entirety. Zcyto20, IFN-λ2 and IL-28A are used interchangeably herein. The IFN-λ2 polypeptides of the present invention include, for example, the polypeptides of SEQ ID NOs:2, 4, 6, 8, 10 and 12.

“zcyto21” is a previous designation for “IL-29” and IL-29 is a previous designation for “Interferon Lambda-1” (IFN-λ1). See, for example, U.S. Pat. Nos. 7,038,032, 6,927,040, 7,135,170, 7,157,559, 7,351,689 and WIPO publication Nos. WO 05/097165, WO 07/012,033, WO 07/013,944 and WO 07/041,713, and all of which are herein incorporated by reference in their entirety. Zcyto21, IFN-λ1 and IL-29 are used interchangeably herein. The IFN-λ1 polypeptides of the present invention include, for example, the polypeptides of SEQ ID NOs:34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 115, 117, 119, 121 and 123.

“zcyto22” is a previous designation for “IL-28B” and IL-28B is a previous designation for “Interferon Lambda-3” (IFN-λ3). See, for example, U.S. Pat. Nos. 7,038,032, 6,927,040, 7,135,170, 7,157,559, 7,351,689 and WIPO publication Nos. WO 05/097165, WO 07/012,033, WO 07/013,944 and WO 07/041,713, and all of which are herein incorporated by reference in their entirety. Zcyto22, IFN-λ3 and IL-28B are used interchangeably herein. The IFN-λ3 polypeptides of the present invention include, for example, the polypeptides of SEQ ID NOs:14, 16, 18, 20, 22, 24, 26, 28, 30 and 32.

“zcytor19” is the previous designation for IL-28 receptor α-subunit or IL-28RA, and is shown in SEQ ID NO:111. The polynucleotides encoding zcytor19 or IL-28RA and the zcytor19 or IL-28RA polypeptides are described in PCT application WO 02/20569 on behalf of Schering, Inc., and WO 02/44209 assigned to ZymoGenetics, Inc., both of which are herein incorporated by reference in their entirety. “IL-28 receptor” denotes the IL-28α-subunit (polypeptide of SEQ ID NO:111) and CRF2-4 subunit (polypeptide of SEQ ID NO:113) forming a heterodimeric receptor.

II. Type III Interferons

The interferon lambdas are a newly described family of cytokines, related to both type-1 Interferons and IL-10 family members. The family, classified as the “Type III” Interferons, is comprised of three recently-identified four helical bundle cytokines designed as IFN-λ1, IFN-λ2 and IFN-λ3 (also referred to as IL-29 or zcyto21, IL-28A or zcyto20, and IL-28B or zcyto22, respectively). Jordan W J et al., Genes and Immunity, 8:13-20 (2007). All three interferon lambdas signal through a heterodimeric receptor complex composed of the class II cytokine receptors IL-28RA (also known as IL-28 receptor alpha) and CRF2-4 (also known as IL-10RB or IL-10R2). The IL-28 receptor is quite distinct from that used by Type I Interferons.

IFN-λ1 is a member of the recently described Type III interferon family (Kotenko S V et al., “IFN-lambdas mediate antiviral protection through a distinct class II cytokine receptor complex”, Nat Immunol 2003; 4(1):69-77; Sheppard P et al., “IL-28, IL-29 and their class II cytokine receptor IL-28R”, Nat Immunol 2003; 4(1):63-68)) with functional similarities to Type I interferons, which include IFN-α and IFN-β (Ank, et al., Journal of Virology, “Lambda interferon (IFN-lambda), a type III IFN, is induced by viruses and IFNs and displays potent antiviral activity against select virus infections in vivo”, 2006; 80(9); 4501-4509). Similarly to IFN-α (which is a Type I interferon), the Type III interferons are induced in response to viral infection and stimulate an intracellular response that involves phosphorylation of signal transducing activator of transcription (STAT) proteins and induction of interferon-responsive genes, also known as interferon stimulated genes (ISGs). ISGs encode proteins involved in antiviral responses and immune stimulation, including Protein kinase R (PkR), Myxovirus resistance (Mx), 2′5′ oligoadenylate synthetase (OAS), and β2-microglobulin (B2M) (Samuel C E. Antiviral actions of interferons. Clin Microbiol Rev 2001; 14(4):778-809; Stark G R, Kerr I M, Williams B R, Silverman R H, Schreiber R D. How cells respond to interferons. Annu Rev Biochem 1998; 67:227-264).

Expression of the IL-28 receptor for the Type III interferons is more restricted than that of the IFN-α receptor. For example, while all cell types in the liver express the IFN-α receptor, the IL-28 receptor for the Type III interferons is found only on hepatocytes. Similarly, in peripheral blood, high levels of the IL-28 receptor for the Type III interferons are detected only on B cells, whereas all peripheral blood leukocytes (PBLs) including B, T, and NK cells, neutrophils, and monocytes express the IFN-α receptor. Consistent with this pattern of receptor expression, treatment of PBLs with the Type III interferons leads to low levels of STAT-1 phosphorylation in B cells but not in other PBLs. This is in contrast to IFN-α, which induces STAT 1 phosphorylation in all PBLs tested.

The present invention provides polynucleotide molecules, including DNA and RNA molecules, which encode an IL-29 or IFN-λ1 polypeptide. For example, the present invention provides degenerate nucleotide sequences encoding IL-29 polypeptides as disclosed herein. Those skilled in the art will readily recognize that, in view of the degeneracy of the genetic code, considerable sequence variation is possible among these polynucleotide molecules. The IL-29 or IFN-λ1 polypeptides of the present invention include, for example, the polypeptides of SEQ ID NOs: 34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 115, 117, 119, 121 and 123, which are encoded by IL-29 or IFN-λ1 polynucleotides as shown in SEQ ID NOs:33, 35, 37, 39, 41, 43, 45, 47, 49, 51, 53, 55, 57, 59, 61, 63, 65, 67, 69, 71, 73, 75, 77, 79, 81, 83, 85, 87, 89, 91, 93, 95, 97, 99, 101, 103, 105, 107, 109, 114, 116, 118, 120 and 122, respectively.

The present invention also provides polynucleotide molecules, including DNA and RNA molecules, which encode an IL-28A or IFN-λ2 polypeptide. For example, the present invention provides degenerate nucleotide sequences encoding IL-28A polypeptides as disclosed herein. Those skilled in the art will readily recognize that, in view of the degeneracy of the genetic code, considerable sequence variation is possible among these polynucleotide molecules. The IL-28A or IFN-λ2 polypeptides of the present invention include, for example, the polypeptides of SEQ ID NOs:2, 4, 6, 8, 10 and 12, which are encoded by IL-28A polynucleotides as shown in SEQ ID NOs:1, 3, 5, 7, 9 and 11, respectively.

The present invention also provides polynucleotide molecules, including DNA and RNA molecules, which encode an IL-28B or IFN-λ3 polypeptide. For example, the present invention provides degenerate nucleotide sequences encoding IL-28B polypeptides as disclosed herein. Those skilled in the art will readily recognize that, in view of the degeneracy of the genetic code, considerable sequence variation is possible among these polynucleotide molecules. The IL-28B or IFN-λ3 polypeptides of the present invention include, for example, the polypeptides of SEQ ID NOs:14, 16, 18, 20, 22, 24, 26, 28, 30 and 32, which are encoded by IL-28B polynucleotides as shown in SEQ ID NOs:13, 15, 17, 19, 21, 23, 25, 27, 29 and 31, respectively.

Table 1 sets forth the one-letter codes used to denote degenerate nucleotide positions. “Resolutions” are the nucleotides denoted by a code letter. “Complement” indicates the code for the complementary nucleotide(s). For example, the code Y denotes either C or T, and its complement R denotes A or G, with A being complementary to T, and G being complementary to C.

TABLE 1

| Nucleotide | Resolution | Complement | Resolution |
|---|---|---|---|
| A | A | T | T |
| C | C | G | G |
| G | G | C | C |
| T | T | A | A |
| R | A\|G | Y | C\|T |
| Y | C\|T | R | A\|G |
| M | A\|C | K | G\|T |
| K | G\|T | M | A\|C |
| S | C\|G | S | C\|G |
| W | A\|T | W | A\|T |
| H | A\|C\|T | D | A\|G\|T |
| B | C\|G\|T | V | A\|C\|G |
| V | A\|C\|G | B | C\|G\|T |
| D | A\|G\|T | H | A\|C\|T |
| N | A\|C\|G\|T | N | A\|C\|G\|T |

The degenerate codons encompass all possible codons for a given amino acid are set forth in Table 2.

TABLE 2

| | One | | |
|---|---|---|---|
| Amino | Letter | | Degenerate |
| Acid | Code | Codons | Codon |
| Cys | C | TGC TGT | TGY |
| Ser | S | AGC AGT TCA TCC TCG TCT | WSN |
| Thr | T | ACA ACC ACG ACT | ACN |
| Pro | P | CCA CCC CCG CCT | CCN |
| Ala | A | GCA GCC GCG GCT | GCN |
| Gly | G | GGA GGC GGG GGT | GGN |
| Asn | N | AAC AAT | AAY |
| Asp | D | GAC GAT | GAY |
| Glu | E | GAA GAG | GAR |
| Gln | Q | CAA CAG | CAR |
| His | H | CAC CAT | CAY |
| Arg | R | AGA AGG CGA CGC CGG CGT | MGN |
| Lys | K | AAA AAG | AAR |
| Met | M | ATG | ATG |
| Ile | I | ATA ATC ATT | ATH |
| Leu | L | CTA CTC CTG CTT TTA TTG | YTN |
| Val | V | GTA GTC GTG GTT | GTN |
| Phe | F | TTC TTT | TTY |
| Tyr | Y | TAC TAT | TAY |
| Trp | W | TGG | TGG |
| Ter | . | TAA TAG TGA | TRR |
| Asn\|Asp | B | | RAY |
| Glu\|Gln | Z | | SAR |
| Any | X | | NNN |

One of ordinary skill in the art will appreciate that some ambiguity is introduced in determining a degenerate codon, representative of all possible codons encoding each amino acid. For example, the degenerate codon for serine (WSN) can, in some circumstances, encode arginine (AGR), and the degenerate codon for arginine (MGN) can, in some circumstances, encode serine (AGY). A similar relationship exists between codons encoding phenylalanine and leucine. Thus, some polynucleotides encompassed by the degenerate sequence may encode variant amino acid sequences, but one of ordinary skill in the art can easily identify such variant sequences by reference to the IL-28A, IL-28B and IL-29 amino acid sequences as disclosed herein. Variant sequences can be readily tested for functionality as described herein.

The isolated polynucleotides of the present invention include, for example, DNA and RNA. Methods for preparing DNA and RNA are well known in the art. In general, RNA is isolated from a tissue or cell that produces large amounts of IL-28A, IL-28B or IL-29 RNA. Such tissues and cells are identified by Northern blotting (Thomas, Proc. Natl. Acad. Sci. USA 77:5201, 1980), or by screening conditioned medium from various cell types for activity on target cells or tissue. Once the activity or RNA producing cell or tissue is identified, total RNA can be prepared using guanidinium isothiocyanate extraction followed by isolation by centrifugation in a CsCl gradient (Chirgwin et al., Biochemistry 18:52-94, 1979). Poly (A)⁺ RNA is prepared from total RNA using the method of Aviv and Leder (Proc. Natl. Acad. Sci. USA 69:1408-12, 1972). Complementary DNA (cDNA) is prepared from poly(A)⁺ RNA using known methods. In the alternative, genomic DNA can be isolated. Polynucleotides encoding IL-28A, IL-28B or IL-29 polypeptides are then identified and isolated by, for example, hybridization or PCR.

A full-length clone encoding an IL-28A, IL-28B or IL-29 polypeptide can be obtained by conventional cloning procedures. See U.S. Pat. No. 7,157,559 and WO 07/041,713. Complementary DNA (cDNA) clones are preferred, although for some applications (e.g., expression in transgenic animals) it may be preferable to use a genomic clone, or to modify a cDNA clone to include at least one genomic intron. Methods for preparing cDNA and genomic clones are well known and within the level of ordinary skill in the art, and include the use of the sequence disclosed herein, or parts thereof, for probing or priming a library. Expression libraries can be probed with antibodies to IL-28 receptor fragments, or other specific binding partners.

IL-28A, IL-28B and IL-29 allelic variants are included in the present invention. Allelic variants of these sequences can be cloned by probing cDNA or genomic libraries from different individuals according to standard procedures. Allelic variants of the DNA sequence include those containing silent mutations and those in which mutations result in amino acid sequence changes, in addition to the cysteine mutations, are within the scope of the present invention, as are proteins which are allelic variants, for example, of SEQ ID NOs:2 (IL-28A), 14 (IL-28B), and 34 (IL-29). cDNAs generated from alternatively spliced mRNAs, which retain the properties of IL-28A, IL-28B or IL-29 polypeptides, are included within the scope of the present invention, as are polypeptides encoded by such cDNAs and mRNAs. Allelic variants and splice variants of these sequences can be cloned by probing cDNA or genomic libraries from different individuals or tissues according to standard procedures known in the art, and mutations to the polynucleotides encoding cysteines or cysteine residues can be introduced as described herein.

IL-28A, IL-28B or IL-29 polypeptides with substantially similar sequence identity are characterized as having one or more amino acid substitutions, deletions or additions. These changes are preferably of a minor nature, that is conservative amino acid substitutions (see Table 3) and other substitutions that do not significantly affect the folding or activity of the polypeptide; small deletions, typically of one to about 30 amino acids; and amino- or carboxyl-terminal extensions, such as an amino-terminal methionine residue, or a small linker peptide of up to about 20-25 residues.

TABLE 3
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Determination of amino acid residues that comprise regions or domains that are critical to maintaining structural integrity can be determined. Within these regions one can determine specific residues that will be more or less tolerant of change and maintain the overall tertiary structure of the molecule. Methods for analyzing sequence structure include, but are not limited to alignment of multiple sequences with high amino acid or nucleotide identity, secondary structure propensities, binary patterns, complementary packing and buried polar interactions (Barton, Current Opin. Struct. Biol. 5:372-376, 1995 and Cordes et al., Current Opin. Struct. Biol. 6:3-10, 1996). In general, when designing modifications to molecules or identifying specific fragments determination of structure will be accompanied by evaluating activity of modified molecules.

Amino acid sequence changes are made in IL-28A, IL-28B and IL-29 polypeptides so as to minimize disruption of higher order structure essential to biological activity. For example, where the IL-28A, IL-28B and IL-29 polypeptide comprises one or more helices, changes in amino acid residues will be made so as not to disrupt the helix geometry and other components of the molecule where changes in conformation abate some critical function, for example, binding of the molecule to its binding partners. The effects of amino acid sequence changes can be predicted by, for example, computer modeling as disclosed above or determined by analysis of crystal structure (see, e.g., Lapthorn et al., Nat. Struct. Biol. 2:266-268, 1995). Other techniques that are well known in the art compare folding of a variant protein to a standard molecule (e.g., the native protein). For example, comparison of the cysteine pattern in a variant and standard molecules can be made. Mass spectrometry and chemical modification using reduction and alkylation provide methods for determining cysteine residues which are associated with disulfide bonds or are free of such associations (Bean et al., Anal. Biochem. 201:216-226, 1992; Gray, Protein Sci. 2:1732-1748, 1993; and Patterson et al., Anal. Chem. 66:3727-3732, 1994). It is generally believed that if a modified molecule does not have the same cysteine pattern as the standard molecule folding would be affected. Another well known and accepted method for measuring folding is circular dichrosism (CD). Measuring and comparing the CD spectra generated by a modified molecule and standard molecule is routine (Johnson, Proteins 7:205-214, 1990). Crystallography is another well known method for analyzing folding and structure. Nuclear magnetic resonance (NMR), digestive peptide mapping and epitope mapping are also known methods for analyzing folding and structurally similarities between proteins and polypeptides (Schaanan et al., Science 257:961-964, 1992).

A Hopp/Woods hydrophilicity profile of the IL-28A, IL-28B and IL-29 polypeptide sequence as shown in IL-28A (SEQ ID NOs:2, 4, 6, 8, 10 and 12), IL-28B (SEQ ID NOs:14, 16, 18, 20, 22, 24, 26, 28, 30 and 32), and IL-29 (SEQ ID NOs:34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 115, 117, 119, 121 and 123) can be generated (Hopp et al., Proc. Natl. Acad. Sci. 78:3824-3828, 1981; Hopp, J. Immun. Meth. 88:1-18, 1986 and Triquier et al., Protein Engineering 11:153-169, 1998). The profile is based on a sliding six-residue window. Buried G, S, and T residues and exposed H, Y, and W residues were ignored. Those skilled in the art will recognize that hydrophilicity or hydrophobicity will be taken into account when designing modifications in the amino acid sequence of a IL-28A, IL-28B and IL-29 polypeptide, so as not to disrupt the overall structural and biological profile. Of particular interest for replacement are hydrophobic residues selected from the group consisting of Val, Leu and Ile or the group consisting of Met, Gly, Ser, Ala, Tyr and Trp.

The identities of essential amino acids can also be inferred from analysis of sequence similarity between IFN-α and members of the family of IL-28A, IL-28B, and IL-29 are disclosed in U.S. Pat. No. 7,157,559. Using methods such as “FASTA” analysis described previously, regions of high similarity are identified within a family of proteins and used to analyze amino acid sequence for conserved regions. An alternative approach to identifying a variant polynucleotide on the basis of structure is to determine whether a nucleic acid molecule encoding a potential variant IL-28A, IL-28B and IL-29 gene can hybridize to a nucleic acid molecule as discussed above.

Other methods of identifying essential amino acids in the polypeptides of the present invention are procedures known in the art, such as site-directed mutagenesis or alanine-scanning mutagenesis (Cunningham and Wells, Science 244:1081 (1989), Bass et al., Proc. Natl. Acad. Sci. USA 88:4498 (1991), Coombs and Corey, “Site-Directed Mutagenesis and Protein Engineering,” in Proteins: Analysis and Design, Angeletti (ed.), pages 259-311 (Academic Press, Inc. 1998)). In the latter technique, single alanine mutations are introduced at every residue in the molecule, and the resultant Cysteine mutant molecules are tested for biological or biochemical activity as disclosed below to identify amino acid residues that are critical to the activity of the molecule. See also, Hilton et al., J. Biol. Chem. 271:4699 (1996).

The IL-28A, IL-28B and IL-29 polypeptides of the present invention can be produced according to conventional techniques using cells comprising an expression vector encoding the polypeptide. As used herein, cells comprising an expression vector include both cells that have been directly manipulated by the introduction of exogenous DNA molecules and progeny thereof that contain the introduced DNA. Suitable host cells are those cell types that can be transformed or transfected with exogenous DNA and grown in culture, and include bacteria, fungal cells, and cultured higher eukaryotic cells. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., eds., Current Protocols in Molecular Biology, John Wiley and Sons, Inc., NY, 1987.

Within another aspect, the present invention provides an expression vector comprising the following operably linked elements: a transcription promoter; a DNA segment encoding an IL-28A, IL-28B or IL-29 polypeptide as described herein; and a transcription terminator.

The present invention also provides an expression vector comprising an isolated and purified DNA molecule including the following operably linked elements: a transcription promoter; a DNA segment encoding a polypeptide comprising an amino acid sequence selected from the group consisting of IL-28A (SEQ ID NOs:2, 4, 6, 8, 10 and 12), IL-28B (SEQ ID NOs:14, 16, 18, 20, 22, 24, 26, 28, 30 and 32), and IL-29 (SEQ ID NOs:34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 115, 117, 119, 121 and 123); and a transcription terminator. The DNA molecule may further comprise a secretory signal sequence operably linked to the DNA segment. The encoding polypeptide may further comprise an affinity tag as described herein. The present invention also provides a cultured cell comprising an expression vector as described herein. The encoded polypeptide has antiviral activity, e.g., hepatitis B and/or hepatitis C.

Within another aspect the present invention provides a cultured cell comprising an expression vector as disclosed herein.

Within another aspect the present invention provides a method of producing a protein comprising culturing a cell comprising an expression vector which comprises the following operably linked elements: a transcription promoter; a DNA segment encoding an IL-28A, IL-28B or IL-29 polypeptide as described herein; and a transcription terminator, under conditions wherein the DNA segment is expressed; and recovering the polypeptide encoded by the DNA segment.

In general, a DNA sequence encoding an IL-28A, IL-28B and IL-29 polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a IL-28A, IL-28B and IL-29 polypeptide into the secretory pathway of a host cell, a secretory signal sequence (also known as a leader sequence, prepro sequence or pre sequence) is provided in the expression vector. The secretory signal sequence can be SEQ ID NOs:119 or 121 of U.S. Pat. No. 7,157,559, amino acid residues 1-21 of SEQ ID NO:2 or SEQ ID NO:7 of U.S. Pat. No. 7,038,032, or may be derived from another secreted protein known to one of skill in the art (e.g., t-PA; see, U.S. Pat. No. 5,641,655) or synthesized de novo. The secretory signal sequence is operably linked to the IL-28A, IL-28B and IL-29 DNA sequence, i.e., the two sequences are joined in the correct reading frame and positioned to direct the newly synthesized polypeptide into the secretory pathway of the host cell. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

A wide variety of suitable recombinant host or cultured cells includes, but is not limited to, gram-negative prokaryotic host organisms. Suitable strains of E. coli include W3110, K12-derived strains MM294, TG-1, JM-107, BL21, and UT5600. Other suitable strains include: BL21(DE3), BL21(DE3)pLysS, BL21(DE3)pLysE, DH1, DH4I, DH5, DH5I, DH51F′, DH51MCR, DH10B, DH10B/p3, DH11S, C600, HB101, JM101, JM105, JM109, JM110, K38, RR1, Y1088, Y1089, CSH18, ER1451, ER1647, E. coli K12, E. coli K12 RV308, E. coli K12 C600, E. coliHB101, E. coli K12 C600 R.sub.k-M.sub.k-, E. coli K12 RR1 (see, for example, Brown (ed.), Molecular Biology Labfax (Academic Press 1991)). In addition, ZGOLD1 and ZGOLD5 are suitable host cells for expressing IL-28A, IL-28B and IL-29 polypeptides of the present invention (see U.S. Patent Publication No. 2008-0096252, which is herein incorporated by reference in its entirety). Other gram-negative prokaryotic hosts can include Serratia, Pseudomonas, Caulobacter. Prokaryotic hosts can include gram-positive organisms such as Bacillus, for example, B. subtilis and B. thuringienesis, and B. thuringienesis var. israelensis, as well as Streptomyces, for example, S. lividans, S. ambofaciens, S. fradiae, and S. griseofuscus. Suitable strains of Bacillus subtilus include BR151, YB886, MI119, MI120, and B170 (see, for example, Hardy, “Bacillus Cloning Methods,” in DNA Cloning: A Practical Approach, Glover (ed.) (IRL Press 1985)). Standard techniques for propagating vectors in prokaryotic hosts are well-known to those of skill in the art (see, for example, Ausubel et al. (eds.), Short Protocols in Molecular Biology, 3^{rd} Edition (John Wiley & Sons 1995); Wu et al., Methods in Gene Biotechnology (CRC Press, Inc. 1997)). In one embodiment, the methods of the present invention use IL-28A, IL-28B and IL-29 expressed in the W3110 strain, which has been deposited at the American Type Culture Collection (ATCC) as ATCC # 27325.

When large scale production of IL-28A, IL-28B and IL-29 using the expression system of the present invention is required, batch fermentation can be used. Generally, batch fermentation comprises that a first stage seed flask is prepared by growing E. coli strains expressing IL-28A, IL-28B and IL-29 in a suitable medium in shake flask culture to allow for growth to an optical density (OD) of between 5 and 20 at 600 nm. A suitable medium would contain nitrogen from a source(s) such as ammonium sulfate, ammonium phosphate, ammonium chloride, yeast extract, hydrolyzed animal proteins, hydrolyzed plant proteins or hydrolyzed caseins. Phosphate will be supplied from potassium phosphate, ammonium phosphate, phosphoric acid or sodium phosphate. Other components would be magnesium chloride or magnesium sulfate, ferrous sulfate or ferrous chloride, and other trace elements. Growth medium can be supplemented with carbohydrates, such as fructose, glucose, galactose, lactose, and glycerol, to improve growth. Alternatively, a fed batch culture is used to generate a high yield of IL-28A, IL-28B and IL-29. The IL-28A, IL-28B and IL-29 producing E. coli strains are grown under conditions similar to those described for the first stage vessel used to inoculate a batch fermentation.

General methods for producing conjugates comprising IL-28A, IL-28B or IL-29, and water-soluble polymer moieties are known in the art. See, for example, Karasiewicz et al., U.S. Pat. No. 5,382,657, Greenwald et al., U.S. Pat. No. 5,738,846, Nieforth et al., Clin. Pharmacol. Ther. 59:636 (1996), Monkarsh et al., Anal. Biochem. 247:434 (1997). PEGylated species can be separated from unconjugated IL-28A, IL-28B and IL-29 polypeptides using standard purification methods, such as dialysis, ultrafiltration, ion exchange chromatography, affinity chromatography, size exclusion chromatography, and the like.

WO 07/041,713 discloses methods of manufacturing IL-29 polypeptides (e.g., SEQ ID NO:106). Specifically, WO 07/041,713 teaches the expression, fermentation, recovery, solubilization of inclusion bodies, clarification and concentration of refolded IL-29 or IFN λ-1, purification, pegylation and purification of pegylated IL-29 or IFN λ-1, and is herein incorporated by reference for such purposes.

Suitable water-soluble polymers include polyethylene glycol (PEG), monomethoxy-PEG, mono-(C1-C10)alkoxy-PEG, aryloxy-PEG, poly-(N-vinyl pyrrolidone)PEG, tresyl monomethoxy PEG, monomethoxy-PEG propionaldehyde, PEG propionaldehyde, bis-succinimidyl carbonate PEG, propylene glycol homopolymers, a polypropylene oxide/ethylene oxide co-polymer, polyoxyethylated polyols (e.g., glycerol), monomethoxy-PEG butyraldehyde, PEG butyraldehyde, monomethoxy-PEG acetaldehyde, PEG acetaldehyde, methoxyl PEG-succinimidyl propionate, methoxyl PEG-succinimidyl butanoate, polyvinyl alcohol, dextran, cellulose, or other carbohydrate-based polymers. A suitable PEG may have a molecular weight from about 600 to about 60,000, including, for example, 5,000 daltons, 12,000 daltons, 20,000 daltons, 30,000 daltons, and 40,000 daltons, which can be linear or branched. An IL-28A, IL-28B and IL-29 conjugate can also comprise a mixture of such water-soluble polymers. U.S. Pat. No. 7,157,559 and WO 07/041,713 teach various types of PEGs and the process for conjugating such PEGs to IL-28A, IL-28B and IL-29 and the process for purifying the PEG-IL-28A, PEG-IL-28B and PEG-IL-29 conjugate.

Clinically, diagnostic tests for HCV include serologic assays for antibodies and molecular tests for viral particles. Enzyme immunoassays are available (Vrielink et al., Transfusion 37:845-849, 1997), but may require confirmation using additional tests such as an immunoblot assay (Pawlotsky et al., Hepatology 27:1700-1702, 1998). Qualitative and quantitative assays generally use polymerase chain reaction techniques, and are preferred for assessing viremia and treatment response (Poynard et al., Lancet 352:1426-1432, 1998; McHutchinson et al., N. Engl. J. Med. 339:1485-1492, 1998). Several commercial tests are available, such as, quantitative RT-PCR (Amplicor HCV Monitor™, Roche Molecular Systems, Branchburg, N.J.) and a branched DNA (deoxyribonucleic acid) signal amplification assay (Quantiplex™ HCV RNA Assay [bDNA], Chiron Corp., Emeryville, Calif.). A patient's HCV RNA can be quantified (for instance, after six months following a “prior treatment” to determine whether the patient has had a viral relapse) to International Units per milliliter, for example, with commercially available real-time PCR assays (e.g., the Abbott RealTime™ HCV assay and the Roche Cobas® TaqMan° HCV assay). See Halfon et al., Journal of Clinical Microbiology, 44(7):2507-2511 (July 2006). A non-specific laboratory test for HCV infection measures alanine aminotransferase level (ALT) and is inexpensive and readily available (National Institutes of Health Consensus Development Conference Panel, Hepatology 26 (Suppl. 1):2S-10S, 1997). Histologic evaluation of liver biopsy is generally considered the most accurate means for determining HCV progression (Yano et al., Hepatology 23:1334-1340, 1996). For a review of clinical tests for HCV, see, Lauer et al., N. Engl. J. Med. 345:41-52, 2001.

A variety of assays known to those skilled in the art can be utilized to detect antibodies which specifically bind to pegylated or nonpegylated IL-28A, IL-28B and IL-29 polypeptides. Exemplary assays are described in detail in Using Antibodies: A Laboratory Manual, Harlow and Lane (Eds.), Cold Spring Harbor Laboratory Press, 1999. Representative examples of such assays include: concurrent immunoelectrophoresis, radio-immunoassays, radio-immunoprecipitations, enzyme-linked immunosorbent assays (ELISA), dot blot assays, Western blot assays, inhibition or competition assays, and sandwich assays.

III. Use of Type III Interferons

For pharmaceutical use, IL-28A, IL-28B and IL-29 polypeptides, which can optionally be conjugated to a polyethylene glycol, are administered to a human patient in accord with known methods to one of skill in the art, such as intravenous administration, e.g., as a bolus or by continuous infusion over a period of time, by intramuscular, intraperitoneal, intracerobrospinal, subcutaneous, intra-articular, intrasynovial, intrathecal, oral, topical, or inhalation routes. In general, pharmaceutical formulations will include a pegylated or nonpegylated IL-28A, IL-28B or IL-29 polypeptide in combination with a pharmaceutically acceptable vehicle, such as saline, buffered saline, 5% dextrose in water, or the like. Formulations may further include one or more excipients, preservatives, solubilizers, buffering agents, albumin to prevent protein loss on vial surfaces, etc. Methods of formulation are well known in the art and are disclosed, for example, in Remington: The Science and Practice of Pharmacy, Gennaro, ed., Mack Publishing Co., Easton, Pa., 19^{th} ed., 1995. In general, a “therapeutically effective amount” is an amount of IL-28A, IL-28B and IL-29 sufficient to produce a clinically significant change in the treated condition, such as a clinically significant change in viral load (e.g., the quantitation of HCV RNA can be determined, as in Example 1, by reverse transcriptase-polymerase chain reaction ((“RT-PCR”) Taqman® as disclosed, for example, in Kleiber et al., “Performance Characteristics of a Quantitative, Homogenous TaqMan RT-PCT Test for HCV RNA”, Journal of Molecular Diagnostics, 2(3):158-166 (August 2000); and Morris et al., “Rapid Reverse Transcription-PCT Detection of Hepatitis C Virus RNA in Serum by Using the TazMan Fluorogenic Detection System,” Journal of Clinical Microbiology, 34(12):2933-2936 (December 1996)) or immune function, a significant reduction in morbidity, or a significantly increased histological score.

For the prevention or treatment of hepatitis C, the fixed dose of the Pegylated Type III Interferon may depend on the severity and course of the disease, whether the Pegylated Type III Interferon is administered for preventive or therapeutic purposes, previous therapy or prior treatment to the patient, the patient's clinical history and response to the Pegylated Type III Interferon, and the discretion of the attending physician. The fixed dose is suitably administered to the patient at one time or over a series of treatments. Preferably, the fixed dose is in the range from about 20 μg to about 800 μg of the Pegylated Type III Interferon. For example, the fixed dose may be about 60-80 μg, about 80-100 μg, about 100-120 μg, about 120-140 μg, about 140-160 μg, about 160-180 μg, about 180-200 μg, about 200-220 μg, about 220-240 μg, about 240-260 μg, about 260-280 μg, or about 280-300 μg of the Pegylated Type III Interferon.

Where a series of fixed doses are administered, these may include, for example, about one dose per week, about two doses per week, about three doses per week, about one dose every other day, about one dose every three days, about one dose every week, about one dose every two weeks, about every 3 weeks, or about every 4 weeks. The fixed doses may, for example, continue to be administered until, for example, the hepatitis C virus is cleared or is unable to be detected, adverse event, or other time as determined by the physician. For example, from about two, three, or four, up to about 48-52 or up to about 100 or more fixed doses may be administered.

In one embodiment, one or more loading dose(s) of the Pegylated Type III Interferon are administered, followed by one or more maintenance dose(s) of the Pegylated Type III Interferon. In another embodiment, a plurality of the same fixed dose are administered to the patient.

In another embodiment, the treatment for the patient may further include, in addition to the Pegylated Type III Interferon, at least one anti-hepatitis C agent. Optionally, the anti-hepatitis C agent is selected from the group consisting of polymerase and/or protease inhibitors, A3AR agonists, Toll-Like Receptor agonists, monoclonal antibodies, Botanicals, anti-phospholipids, immunomodulators, anti-inflammatory drugs, thiazolides, broad spectrum immune stimulators, inflammatory/fibrosis inhibitors, cyclophilin inhibitors, pancaspase inhibitors, HCV immune globulins, antivirals, anti-infectives, RNA inhibitors, glucosidase I inhibitors, IRES inhibitors, bezafibrates, nucleoside analogs, Type I Interferons and Type II Interferons. The polymerase and/or protease inhibitor can be, for example, VCH-916 (Virochem), GS9190 (Gilead), GSK625433 (GlaxcoSmithKline), ITMN-191 (R-7227; InterMune), R7128 (Pharmasset/Roche), VCH-759 (Virochem), R1626 (Roche), TMC435350 (Medivir/Tibotec), SCH503034 (Boceprevir, Schering-Plough), A-831 (Arrow Therapeutics), valopicitabine (NM283, Idenix Pharmaceuticals) or VX950 (Telaprevir, Vertex). The A3AR agonist can be, for example, CF102 (Can-Fite). The Toll-Like Receptor agonist can be, for example, IMO-2125 (Idera Pharmaceuticals), Isatoribine (ANA971, Anadys Pharmaceuticals) or Actilon (CPG10101, Coley Pharmaceutical Group). The monoclonal antibody can be, for example, AB68 (XTL bio). The Botanical can be, for example, PYN17 (Phynova). The anti-phospholipid can be, for example, Bavituximab (formerly Tarvacin; Peregrine). The immunomodulator can be, for example, NOV-205 (Novelos Therapeutics), Oglufanide disodium (Implicit Bioscience) or thymalfasin (thymosin alpha 1; SciClone/Sigma-Tau). The anti-inflammatory drug can be, for example, CTS-1027 (Conatus) or JBK-122 (Jenken Biosciences). The thiazolides can be, for example, Alinia (nitazoxanide; Romark Laboratories). The broad spectrum immune stimulator can be, for example, SCV-07 (SciClone). The inflammatory/fibrosis inhibitor can be, for example, MitoQ (mitoquinone; Antipodean Pharmaceuticals). The cyclophilin inhibitor can be, for example, DEBIO-025 (Debio Pharm Group). The pancaspase inhibitor can be, for example, PF-03491390 (formerly IDN-6556; Pfizer Pharmaceuticals). The HCV immune globulin can be, for example, Civacir (Nabi). The antiviral can be, for example, Suvus (Methylene blue, formerly BIVN-104 (Virostat); Bioenvision). Optionally, the anti-infective is Nitazoxanide (Alinia®, Romark Pharmaceuticals). The glucosidase I inhibitor can be, for example, MX-3253 (celgosivir; Migenix). The IRES inhibitor can be, for example, VGX-410C (Mifepristone; VGX Pharmaceuticals). The bezafibrate can be, for example, Hepaconda (Giaconda). The nucleoside analog can be, for example, ribavirin (Roches's Copegus or Schering-Plough's Rebetol) or viramidine (taribavirin (ribavirin pro-drug); Valeant Pharmaceuticals). Optionally, the ribavirin or viramidine is administered orally once or twice daily to the patient at a dose of about 800-1200 mg. The Type I Interferon can be, for example, Interferon alpha or pegylated Interferon alpha. Optionally, the Interferon alpha or pegylated Interferon alpha is PEGASYS (pegylated interferon-alpha-2a or peg-IFN-α-2a; Roche), PEG-INTRON (pegylated interferon-alpha-2b or peg-IFN-α-2b; Schering-Plough), Belerofon (Nautilus Biotech), oral interferon alpha (Amarillo Biosciences), BLX-883 (Locteron; Biolex Therapeutics/OctoPlus), Multiferon (Viragen), Albuferon (Human Genome Sciences), Consensus Interferon or (Infergen; Three Rivers Pharma). The Type I Interferon can be, for example, omega interferon (Intarcia Therapeutics). Optionally, the Type II Interferon is Interferon gamma, e.g., Actimmune® by Intermune. The polyethylene glycol (PEG) of the pegylated Type III Interferon can be, for example, 20 kD, 30 kD or 40 kD mPEG-propionaldehyde. The 20 kD, 30 kD or 40 kD mPEG-propionaldehyde can be conjugated, for example, to the N-terminus of the Type III Interferon polypeptide.

Suitable dosages for any of the above coadministered agents are those presently used and may be lowered due to the combined action (synergy) of the anti-hepatitis C agent and the Pegylated Type III Interferon.

As an illustration, pharmaceutical formulations may be supplied as a kit comprising a container that comprises a pegylated or nonpegylated IL-28A, IL-28B or IL-29 polypeptide of the present invention. The kit may further comprise an anti-hepatitis C agent as described herein. Therapeutic polypeptides can be provided in the form of an injectable solution for single or multiple doses, or as a sterile powder that can be reconstituted before injection. Alternatively, such a kit can include a dry-powder disperser, liquid aerosol generator, or nebulizer for administration of a therapeutic polypeptide. Such a kit may further comprise written information on indications and usage of the pharmaceutical formulation. Moreover, such information may include a statement that the pegylated or nonpegylated IL-28A, IL-28B or IL-29 polypeptide formulation is contraindicated in patients with known hypersensitivity to pegylated or nonpegylated IL-28A, IL-28B and/or IL-29 polypeptide.

The present invention provides for a method of treating a human patient infected or at risk of infection with the hepatitis C virus comprising administering to the human patient a therapeutically effective amount of a Pegylated Type III Interferon or Type III Interferon. Optionally, the dose can be one dose per week, two doses per week, three doses per week, one dose every other day, one dose every three days, or one dose every two weeks. Optionally, the Pegylated Type III Interferon or Type III Interferon can be IL-28A polypeptide, an IL-28B polypeptide, or an IL-29 polypeptide. The IL-28A polypeptide can be, for example, the polypeptide of SEQ ID NOs:2, 4, 6, 8, 10 or 12. The IL-28B polypeptide can be, for example, the polypeptide of SEQ ID NOs:14, 16, 18, 20, 22, 24, 26, 28, 30 or 32. The IL-29 polypeptide can be, for example, the polypeptide of SEQ ID NOs:34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 115, 117, 119, 121 or 123. The Pegylated Type III Interferon or Type III Interferon can be administered parenterally, such as by injection or infusion. The Pegylated Type III Interferon or Type III Interferon can be administered intravenously, intramuscularly, subcutaneously, intradermally, or intraperitoneally. Optionally, the Pegylated Type III Interferon or Type III Interferon is administered to the human patient in an amount selected from the group consisting of less than 0.5 μg/kg, 0.5 to 1.0 μg/kg, 1.0 to 1.5 μg/kg, 1.5 to 2.0 μg/kg, 2.0 to 2.5 μg/kg, 2.5 to 3.0 μg/kg, 3.0 to 3.5 μg/kg, 3.5 to 4.0 μg/kg, 4.0 to 4.5 μg/kg, 4.5 to 5.0 μg/kg, 5.0 to 5.5 μg/kg, 5.5 to 6.0 μg/kg, 6.0 to 6.5 μg/kg, 6.5 to 7.0 μg/kg, 7.0 to 7.5 μg/kg, 7.5 to 8.0 μg/kg, 8.0 to 8.5 μg/kg, 8.5 to 9.0 μg/kg, 9.0 to 9.5 μg/kg, 9.5 to 10.0 μg/kg, greater than 10.0 μg/kg, fixed dose of about 60-80 μg, fixed dose of about 80-100 μg, fixed dose of about 100-120 μg, fixed dose of about 120-140 μg, fixed dose of about 140-160 μg, fixed dose of about 160-180 μg, fixed dose of about 180-200 μg, fixed dose of about 200-220 μg, fixed dose of about 220-240 μg, fixed dose of about 240-260 μg, fixed dose of about 260-280 μg, and fixed dose of about 280-300 μg.

Optionally, the human patient having HCV is selected from a subpopulation of hepatitis C patients consisting of treatment naïve patients with genotype I hepatitis C; treatment naïve patients with any hepatitis C genotype (e.g., 1a, 1b, 1c, 2a, 2b, 2c, 3a, 3b, 4a, 4b, 4c, 4d, 4e, 5a, 6a, 7a, 7b, 8a, 8b, 9a, 10a, and 11a); patients co-infected with the human immunodeficiency virus (HIV); patients intolerant to Pegylated Interferon Alpha, Interferon Alpha or any other Pegylated or NonPegylated Type I Interferon; patients for whom treatment with Pegylated Interferon Alpha, Interferon Alpha or any other Pegylated or NonPegylated Type I Interferon is contraindicated; patients awaiting or following liver transplant; patients with decompensated liver disease; patients who are previous non-responders to treatment with Pegylated Interferon Alpha, Interferon Alpha or any other Pegylated or NonPegylated Type I Interferon either as a single agent or in combination with ribavirin or any other anti-hepatitis C agent, including patients who were null responders, responder/relapsers, or break-through patients; patients who were non-compliant with prior treatment with Pegylated Interferon Alpha, Interferon Alpha or any other Pegylated or NonPegylated Type I Interferon either as a single agent or in combination with ribavirin or other any of the anti-hepatitis C agents; patients with any base level of hepatitis C RNA; and patients with cirrhosis. Optionally, the duration of the treatment is 8-12 weeks, 12-16 weeks, 16-20 weeks, 20-24 weeks, 24-28 weeks, 28-32 weeks, 32-36 weeks, 36-40 weeks, 40-44 weeks, 44-48 weeks, 48-52 weeks, or greater than 52 weeks. Optionally, the treatment can further include at least one anti-hepatitis C agent. Optionally, the anti-hepatitis C agent is selected from the group consisting of polymerase and/or protease inhibitors, A3AR agonists, Toll-Like Receptor agonists, monoclonal antibodies, Botanicals, anti-phospholipids, immunomodulators, anti-inflammatory drugs, thiazolides, broad spectrum immune stimulators, inflammatory/fibrosis inhibitors, cyclophilin inhibitors, pancaspase inhibitors, HCV immune globulins, antivirals, anti-infectives, RNA inhibitors, glucosidase I inhibitors, IRES inhibitors, bezafibrates, nucleoside analogs, Type I Interferons and Type II Interferons. The polymerase and/or protease inhibitor can be, for example, VCH-916 (Virochem), GS9190 (Gilead), GSK625433 (GlaxcoSmithKline), ITMN-191 (R-7227; InterMune), R7128 (Pharmasset/Roche), VCH-759 (Virochem), R1626 (Roche), TMC435350 (Medivir/Tibotec), SCH503034 (Boceprevir, Schering-Plough), A-831 (Arrow Therapeutics), valopicitabine (NM283, Idenix Pharmaceuticals) or VX950 (Telaprevir, Vertex). The A3AR agonist can be, for example, CF102 (Can-Fite). The Toll-Like Receptor agonist can be, for example, IMO-2125 (Idera Pharmaceuticals), Isatoribine (ANA971, Anadys Pharmaceuticals) or Actilon (CPG10101, Coley Pharmaceutical Group). The monoclonal antibody can be, for example, AB68 (XTL bio). The Botanical can be, for example, PYN17 (Phynova). The anti-phospholipid can be, for example, Bavituximab (formerly Tarvacin; Peregrine). The immunomodulator can be, for example, NOV-205 (Novelos Therapeutics), Oglufanide disodium (Implicit Bioscience) or thymalfasin (thymosin alpha 1; SciClone/Sigma-Tau). The anti-inflammatory drug can be, for example, CTS-1027 (Conatus) or JBK-122 (Jenken Biosciences). The thiazolides can be, for example, Alinia (nitazoxanide; Romark Laboratories). The broad spectrum immune stimulator can be, for example, SCV-07 (SciClone). The inflammatory/fibrosis inhibitor can be, for example, MitoQ (mitoquinone; Antipodean Pharmaceuticals). The cyclophilin inhibitor can be, for example, DEBIO-025 (Debio Pharm Group). The pancaspase inhibitor can be, for example, PF-03491390 (formerly IDN-6556; Pfizer Pharmaceuticals). The HCV immune globulin can be, for example, Civacir (Nabi). The antiviral can be, for example, Suvus (Methylene blue, formerly BIVN-104 (Virostat); Bioenvision). Optionally, the anti-infective is Nitazoxanide (Alinia®, Romark Pharmaceuticals). The glucosidase I inhibitor can be, for example, MX-3253 (celgosivir; Migenix). The IRES inhibitor can be, for example, VGX-410C (Mifepristone; VGX Pharmaceuticals). The bezafibrate can be, for example, Hepaconda (Giaconda). The nucleoside analog can be, for example, ribavirin (Roches's Copegus or Schering-Plough's Rebetol) or viramidine (taribavirin (ribavirin pro-drug); Valeant Pharmaceuticals). Optionally, the ribavirin or viramidine is administered orally once or twice daily to the patient at a dose of about 800-1200 mg. The Type I Interferon can be, for example, Interferon alpha or pegylated Interferon alpha. Optionally, the Interferon alpha or pegylated Interferon alpha is PEGASYS (pegylated interferon-alpha-2a or peg-IFN-α-2a; Roche), PEG-INTRON (pegylated interferon-alpha-2b or peg-IFN-α-2b; Schering-Plough), Belerofon (Nautilus Biotech), oral interferon alpha (Amarillo Biosciences), BLX-883 (Locteron; Biolex Therapeutics/OctoPlus), Multiferon (Viragen), Albuferon (Human Genome Sciences), Consensus Interferon or (Infergen; Three Rivers Pharma). The Type I Interferon can be, for example, omega interferon (Intarcia Therapeutics). Optionally, the Type II Interferon is Interferon gamma, e.g., Actimmune® by Intermune. The polyethylene glycol (PEG) of the pegylated Type III Interferon can be, for example, 20 kD, 30 kD or 40 kD mPEG-propionaldehyde. The 20 kD, 30 kD or 40 kD mPEG-propionaldehyde can be conjugated, for example, to the N-terminus of the Type III Interferon polypeptide.

The present invention also provides for a method of treating a human patient infected or at risk of infection with the hepatitis C virus comprising administering to the human patient a therapeutically effective amount of a pharmaceutical formulation comprising a Pegylated Type III Interferon or a Type III Interferon and a pharmaceutically acceptable vehicle. Optionally, the dose can be one dose per week, two doses per week, three doses per week, one dose every other day, one dose every three days, or one dose every two weeks. Optionally, the Type III Interferon can be IL-28A polypeptide, an IL-28B polypeptide, or an IL-29 polypeptide. The IL-28A polypeptide can be, for example, the polypeptide of SEQ ID NOs:2, 4, 6, 8, 10 or 12. The IL-28B polypeptide can be, for example, the polypeptide of SEQ ID NOs:14, 16, 18, 20, 22, 24, 26, 28, 30 or 32. The IL-29 polypeptide can be, for example, the polypeptide of SEQ ID NOs:34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 115, 117, 119, 121 or 123. The Pegylated Type III Interferon or Type III Interferon can be administered parenterally, such as by injection or infusion. The Pegylated Type III Interferon or Type III Interferon can be administered intravenously, intramuscularly, subcutaneously, intradermally, or intraperitoneally. Optionally, the Pegylated Type III Interferon or Type III Interferon is administered to the human patient in an amount selected from the group consisting of less than 0.5 μg/kg, 0.5 to 1.0 μg/kg, 1.0 to 1.5 μg/kg, 1.5 to 2.0 μg/kg, 2.0 to 2.5 μg/kg, 2.5 to 3.0 μg/kg, 3.0 to 3.5 μg/kg, 3.5 to 4.0 μg/kg, 4.0 to 4.5 μg/kg, 4.5 to 5.0 μg/kg, 5.0 to 5.5 μg/kg, 5.5 to 6.0 μg/kg, 6.0 to 6.5 μg/kg, 6.5 to 7.0 μg/kg, 7.0 to 7.5 μg/kg, 7.5 to 8.0 μg/kg, 8.0 to 8.5 μg/kg, 8.5 to 9.0 μg/kg, 9.0 to 9.5 μg/kg, 9.5 to 10.0 μg/kg, greater than 10.0 μg/kg, fixed dose of about 60-80 μg, fixed dose of about 80-100 μg, fixed dose of about 100-120 μg, fixed dose of about 120-140 μg, fixed dose of about 140-160 μg, fixed dose of about 160-180 μg, fixed dose of about 180-200 μg, fixed dose of about 200-220 μg, fixed dose of about 220-240 μg, fixed dose of about 240-260 μg, fixed dose of about 260-280 μg, and fixed dose of about 280-300 μg. Optionally, the human patient having HCV is selected from a subpopulation of hepatitis C patients consisting of treatment naïve patients with genotype I hepatitis C; treatment naïve patients with any hepatitis C genotype; patients co-infected with the human immunodeficiency virus (HIV); patients intolerant to Pegylated Interferon Alpha, Interferon Alpha or any other Pegylated or NonPegylated Type I Interferon; patients for whom treatment with Pegylated Interferon Alpha, Interferon Alpha or any other Pegylated or NonPegylated Type I Interferon is contraindicated; patients awaiting or following liver transplant; patients with decompensated liver disease; patients who are previous non-responders to treatment with Pegylated Interferon Alpha, Interferon Alpha or any other Pegylated or NonPegylated Type I Interferon either as a single agent or in combination with ribavirin or any other anti-hepatitis C agent, including patients who were null responders, responder/relapsers, or break-through patients; patients who were non-compliant with prior treatment with Pegylated Interferon Alpha, Interferon Alpha or any other Pegylated or NonPegylated Type I Interferon either as a single agent or in combination with ribavirin or other any of the anti-hepatitis C agents; patients with any base level of hepatitis C RNA; and patients with cirrhosis. Optionally, the duration of the treatment is 8-12 weeks, 12-16 weeks, 16-20 weeks, 20-24 weeks, 24-28 weeks, 28-32 weeks, 32-36 weeks, 36-40 weeks, 40-44 weeks, 44-48 weeks, 48-52 weeks, or greater than 52 weeks. Optionally, the treatment can further include at least one anti-hepatitis C agent. Optionally, the anti-hepatitis C agent is selected from the group consisting of polymerase and/or protease inhibitors, A3AR agonists, Toll-Like Receptor agonists, monoclonal antibodies, Botanicals, anti-phospholipids, immunomodulators, anti-inflammatory drugs, thiazolides, broad spectrum immune stimulators, inflammatory/fibrosis inhibitors, cyclophilin inhibitors, pancaspase inhibitors, HCV immune globulins, antivirals, anti-infectives, RNA inhibitors, glucosidase I inhibitors, IRES inhibitors, bezafibrates, nucleoside analogs, Type I Interferons and Type II Interferons. The polymerase and/or protease inhibitor can be, for example, VCH-916 (Virochem), GS9190 (Gilead), GSK625433 (GlaxcoSmithKline), ITMN-191 (R-7227; InterMune), R7128 (Pharmasset/Roche), VCH-759 (Virochem), R1626 (Roche), TMC435350 (Medivir/Tibotec), SCH503034 (Boceprevir, Schering-Plough), A-831 (Arrow Therapeutics), valopicitabine (NM283, Idenix Pharmaceuticals) or VX950 (Telaprevir, Vertex). The A3AR agonist can be, for example, CF102 (Can-Fite). The Toll-Like Receptor agonist can be, for example, IMO-2125 (Idera Pharmaceuticals), Isatoribine (ANA971, Anadys Pharmaceuticals) or Actilon (CPG10101, Coley Pharmaceutical Group). The monoclonal antibody can be, for example, AB68 (XTL bio). The Botanical can be, for example, PYN17 (Phynova). The anti-phospholipid can be, for example, Bavituximab (formerly Tarvacin; Peregrine). The immunomodulator can be, for example, NOV-205 (Novelos Therapeutics), Oglufanide disodium (Implicit Bioscience) or thymalfasin (thymosin alpha 1; SciClone/Sigma-Tau). The anti-inflammatory drug can be, for example, CTS-1027 (Conatus) or JBK-122 (Jenken Biosciences). The thiazolides can be, for example, Alinia (nitazoxanide; Romark Laboratories). The broad spectrum immune stimulator can be, for example, SCV-07 (SciClone). The inflammatory/fibrosis inhibitor can be, for example, MitoQ (mitoquinone; Antipodean Pharmaceuticals). The cyclophilin inhibitor can be, for example, DEBIO-025 (Debio Pharm Group). The pancaspase inhibitor can be, for example, PF-03491390 (formerly IDN-6556; Pfizer Pharmaceuticals). The HCV immune globulin can be, for example, Civacir (Nabi). The antiviral can be, for example, Suvus (Methylene blue, formerly BIVN-104 (Virostat); Bioenvision). Optionally, the anti-infective is Nitazoxanide (Alinia®, Romark Pharmaceuticals). The glucosidase I inhibitor can be, for example, MX-3253 (celgosivir; Migenix). The IRES inhibitor can be, for example, VGX-410C (Mifepristone; VGX Pharmaceuticals). The bezafibrate can be, for example, Hepaconda (Giaconda). The nucleoside analog can be, for example, ribavirin (Roches's Copegus or Schering-Plough's Rebetol) or viramidine (taribavirin (ribavirin pro-drug); Valeant Pharmaceuticals). Optionally, the ribavirin or viramidine is administered orally once or twice daily to the patient at a dose of about 800-1200 mg. The Type I Interferon can be, for example, Interferon alpha or pegylated Interferon alpha. Optionally, the Interferon alpha or pegylated Interferon alpha is PEGASYS (pegylated interferon-alpha-2a or peg-IFN-α-2a; Roche), PEG-INTRON (pegylated interferon-alpha-2b or peg-IFN-α-2b; Schering-Plough), Belerofon (Nautilus Biotech), oral interferon alpha (Amarillo Biosciences), BLX-883 (Locteron; Biolex Therapeutics/OctoPlus), Multiferon (Viragen), Albuferon (Human Genome Sciences), Consensus Interferon or (Infergen; Three Rivers Pharma). The Type I Interferon can be, for example, omega interferon (Intarcia Therapeutics). Optionally, the Type II Interferon is Interferon gamma, e.g., Actimmune® by Intermune. The polyethylene glycol (PEG) of the pegylated Type III Interferon can be, for example, 20 kD, 30 kD or 40 kD mPEG-propionaldehyde. The 20 kD, 30 kD or 40 kD mPEG-propionaldehyde can be conjugated, for example, to the N-terminus of the Type III Interferon polypeptide.

The present invention also provides for a method of treating a human patient having a relapsing genotype I chronic hepatitis C infection following prior treatment comprising administering to the human patient a therapeutically effective amount of a Pegylated Type III Interferon or Type III Interferon. Optionally, the dose can be, for example, one dose per week, two doses per week, three doses per week, one dose every other day, one dose every three days, or one dose every two weeks. Optionally, the Type III Interferon can be IL-28A polypeptide, an IL-28B polypeptide, or an IL-29 polypeptide. The IL-28A polypeptide can be, for example, the polypeptide of SEQ ID NOs:2, 4, 6, 8, 10 or 12. The IL-28B polypeptide can be, for example, the polypeptide of SEQ ID NOs:14, 16, 18, 20, 22, 24, 26, 28, 30 or 32. The IL-29 polypeptide can be, for example, the polypeptide of SEQ ID NOs:34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 115, 117, 119, 121 or 123. The Pegylated Type III Interferon or Type III Interferon can be administered parenterally, such as by injection or infusion. The Pegylated Type III Interferon or Type III Interferon can be administered intravenously, intramuscularly, subcutaneously, intradermally, or intraperitoneally. Optionally, the Pegylated Type III Interferon or Type III Interferon is administered to the human patient in an amount selected from the group consisting of less than 0.5 μg/kg, 0.5 to 1.0 μg/kg, 1.0 to 1.5 μg/kg, 1.5 to 2.0 μg/kg, 2.0 to 2.5 μg/kg, 2.5 to 3.0 μg/kg, 3.0 to 3.5 μg/kg, 3.5 to 4.0 μg/kg, 4.0 to 4.5 μg/kg, 4.5 to 5.0 μg/kg, 5.0 to 5.5 μg/kg, 5.5 to 6.0 μg/kg, 6.0 to 6.5 μg/kg, 6.5 to 7.0 μg/kg, 7.0 to 7.5 μg/kg, 7.5 to 8.0 μg/kg, 8.0 to 8.5 μg/kg, 8.5 to 9.0 μg/kg, 9.0 to 9.5 μg/kg, 9.5 to 10.0 μg/kg, greater than 10.0 μg/kg, fixed dose of about 60-80 μg, fixed dose of about 80-100 μg, fixed dose of about 100-120 μg, fixed dose of about 120-140 μg, fixed dose of about 140-160 μg, fixed dose of about 160-180 μg, fixed dose of about 180-200 μg, fixed dose of about 200-220 μg, fixed dose of about 220-240 μg, fixed dose of about 240-260 μg, fixed dose of about 260-280 μg, and fixed dose of about 280-300 μg. Optionally, the duration of the treatment is 8-12 weeks, 12-16 weeks, 16-20 weeks, 20-24 weeks, 24-28 weeks, 28-32 weeks, 32-36 weeks, 36-40 weeks, 40-44 weeks, 44-48 weeks, 48-52 weeks, or greater than 52 weeks. Optionally, the treatment can further include at least one anti-hepatitis C agent. Optionally, the anti-hepatitis C agent is selected from the group consisting of polymerase and/or protease inhibitors, A3AR agonists, Toll-Like Receptor agonists, monoclonal antibodies, Botanicals, anti-phospholipids, immunomodulators, anti-inflammatory drugs, thiazolides, broad spectrum immune stimulators, inflammatory/fibrosis inhibitors, cyclophilin inhibitors, pancaspase inhibitors, HCV immune globulins, antivirals, anti-infectives, RNA inhibitors, glucosidase I inhibitors, IRES inhibitors, bezafibrates, nucleoside analogs, Type I Interferons and Type II Interferons. The polymerase and/or protease inhibitor can be, for example, VCH-916 (Virochem), GS9190 (Gilead), GSK625433 (GlaxcoSmithKline), ITMN-191 (R-7227; InterMune), R7128 (Pharmasset/Roche), VCH-759 (Virochem), R1626 (Roche), TMC435350 (Medivir/Tibotec), SCH503034 (Boceprevir, Schering-Plough), A-831 (Arrow Therapeutics), valopicitabine (NM283, Idenix Pharmaceuticals) or VX950 (Telaprevir, Vertex). The A3AR agonist can be, for example, CF102 (Can-Fite). The Toll-Like Receptor agonist can be, for example, IMO-2125 (Idera Pharmaceuticals), Isatoribine (ANA971, Anadys Pharmaceuticals) or Actilon (CPG10101, Coley Pharmaceutical Group). The monoclonal antibody can be, for example, AB68 (XTL bio). The Botanical can be, for example, PYN17 (Phynova). The anti-phospholipid can be, for example, Bavituximab (formerly Tarvacin; Peregrine). The immunomodulator can be, for example, NOV-205 (Novelos Therapeutics), Oglufanide disodium (Implicit Bioscience) or thymalfasin (thymosin alpha 1; SciClone/Sigma-Tau). The anti-inflammatory drug can be, for example, CTS-1027 (Conatus) or JBK-122 (Jenken Biosciences). The thiazolides can be, for example, Alinia (nitazoxanide; Romark Laboratories). The broad spectrum immune stimulator can be, for example, SCV-07 (SciClone). The inflammatory/fibrosis inhibitor can be, for example, MitoQ (mitoquinone; Antipodean Pharmaceuticals). The cyclophilin inhibitor can be, for example, DEBIO-025 (Debio Pharm Group). The pancaspase inhibitor can be, for example, PF-03491390 (formerly IDN-6556; Pfizer Pharmaceuticals). The HCV immune globulin can be, for example, Civacir (Nabi). The antiviral can be, for example, Suvus (Methylene blue, formerly BIVN-104 (Virostat); Bioenvision). Optionally, the anti-infective is Nitazoxanide (Alinia®, Romark Pharmaceuticals). The glucosidase I inhibitor can be, for example, MX-3253 (celgosivir; Migenix). The IRES inhibitor can be, for example, VGX-410C (Mifepristone; VGX Pharmaceuticals). The bezafibrate can be, for example, Hepaconda (Giaconda). The nucleoside analog can be, for example, ribavirin (Roches's Copegus or Schering-Plough's Rebetol) or viramidine (taribavirin (ribavirin pro-drug); Valeant Pharmaceuticals). Optionally, the ribavirin or viramidine is administered orally once or twice daily to the patient at a dose of about 800-1200 mg. The Type I Interferon can be, for example, Interferon alpha or pegylated Interferon alpha. Optionally, the Interferon alpha or pegylated Interferon alpha is PEGASYS (pegylated interferon-alpha-2a or peg-IFN-α-2a; Roche), PEG-INTRON (pegylated interferon-alpha-2b or peg-IFN-α-2b; Schering-Plough), Belerofon (Nautilus Biotech), oral interferon alpha (Amarillo Biosciences), BLX-883 (Locteron; Biolex Therapeutics/OctoPlus), Multiferon (Viragen), Albuferon (Human Genome Sciences), Consensus Interferon or (Infergen; Three Rivers Pharma). The Type I Interferon can be, for example, omega interferon (Intarcia Therapeutics). Optionally, the Type II Interferon is Interferon gamma, e.g., Actimmune® by Intermune. The polyethylene glycol (PEG) of the pegylated Type III Interferon can be, for example, 20 kD, 30 kD or 40 kD mPEG-propionaldehyde. The 20 kD, 30 kD or 40 kD mPEG-propionaldehyde can be conjugated, for example, to the N-terminus of the Type III Interferon polypeptide.

The present invention also provides for a method of treating a human patient having a relapsing genotype I chronic hepatitis C infection following prior treatment comprising administering to the human patient a therapeutically effective amount of a pharmaceutical formulation comprising a Pegylated Type III Interferon or a Type III Interferon and a pharmaceutically acceptable vehicle. Optionally, the dose can be, for example, one dose per week, two doses per week, three doses per week, one dose every other day, one dose every three days, or one dose every two weeks. Optionally, the Pegylated Type III Interferon or Type III Interferon can be IL-28A polypeptide, an IL-28B polypeptide, or an IL-29 polypeptide. The IL-28A polypeptide can be, for example, the polypeptide of SEQ ID NOs:2, 4, 6, 8, 10 or 12. The IL-28B polypeptide can be, for example, the polypeptide of SEQ ID NOs:14, 16, 18, 20, 22, 24, 26, 28, 30 or 32. The IL-29 polypeptide can be, for example, the polypeptide of SEQ ID NOs:34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 115, 117, 119, 121 or 123. The Pegylated Type III Interferon or Type III Interferon can be administered parenterally, such as by injection or infusion. The Pegylated Type III Interferon or Type III Interferon can be administered intravenously, intramuscularly, subcutaneously, intradermally, or intraperitoneally. Optionally, the Pegylated Type III Interferon or Type III Interferon is administered to the human patient in an amount selected from the group consisting of less than 0.5 μg/kg, 0.5 to 1.0 μg/kg, 1.0 to 1.5 μg/kg, 1.5 to 2.0 μg/kg, 2.0 to 2.5 μg/kg, 2.5 to 3.0 μg/kg, 3.0 to 3.5 μg/kg, 3.5 to 4.0 μg/kg, 4.0 to 4.5 μg/kg, 4.5 to 5.0 μg/kg, 5.0 to 5.5 μg/kg, 5.5 to 6.0 μg/kg, 6.0 to 6.5 μg/kg, 6.5 to 7.0 μg/kg, 7.0 to 7.5 μg/kg, 7.5 to 8.0 μg/kg, 8.0 to 8.5 μg/kg, 8.5 to 9.0 μg/kg, 9.0 to 9.5 μg/kg, 9.5 to 10.0 μg/kg, greater than 10.0 μg/kg, fixed dose of about 60-80 μg, fixed dose of about 80-100 μg, fixed dose of about 100-120 μg, fixed dose of about 120-140 μg, fixed dose of about 140-160 μg, fixed dose of about 160-180 μg, fixed dose of about 180-200 μg, fixed dose of about 200-220 μg, fixed dose of about 220-240 μg, fixed dose of about 240-260 μg, fixed dose of about 260-280 μg, and fixed dose of about 280-300 μg. Optionally, the duration of the treatment is 8-12 weeks, 12-16 weeks, 16-20 weeks, 20-24 weeks, 24-28 weeks, 28-32 weeks, 32-36 weeks, 36-40 weeks, 40-44 weeks, 44-48 weeks, 48-52 weeks, or greater than 52 weeks. Optionally, the treatment can further include at least one anti-hepatitis C agent. Optionally, the anti-hepatitis C agent is selected from the group consisting of polymerase and/or protease inhibitors, A3AR agonists, Toll-Like Receptor agonists, monoclonal antibodies, Botanicals, anti-phospholipids, immunomodulators, anti-inflammatory drugs, thiazolides, broad spectrum immune stimulators, inflammatory/fibrosis inhibitors, cyclophilin inhibitors, pancaspase inhibitors, HCV immune globulins, antivirals, anti-infectives, RNA inhibitors, glucosidase I inhibitors, IRES inhibitors, bezafibrates, nucleoside analogs, Type I Interferons and Type II Interferons. The polymerase and/or protease inhibitor can be, for example, VCH-916 (Virochem), GS9190 (Gilead), GSK625433 (GlaxcoSmithKline), ITMN-191 (R-7227; InterMune), R7128 (Pharmasset/Roche), VCH-759 (Virochem), R1626 (Roche), TMC435350 (Medivir/Tibotec), SCH503034 (Boceprevir, Schering-Plough), A-831 (Arrow Therapeutics), valopicitabine (NM283, Idenix Pharmaceuticals) or VX950 (Telaprevir, Vertex). The A3AR agonist can be, for example, CF102 (Can-Fite). The Toll-Like Receptor agonist can be, for example, IMO-2125 (Idera Pharmaceuticals), Isatoribine (ANA971, Anadys Pharmaceuticals) or Actilon (CPG10101, Coley Pharmaceutical Group). The monoclonal antibody can be, for example, AB68 (XTL bio). The Botanical can be, for example, PYN17 (Phynova). The anti-phospholipid can be, for example, Bavituximab (formerly Tarvacin; Peregrine). The immunomodulator can be, for example, NOV-205 (Novelos Therapeutics), Oglufanide disodium (Implicit Bioscience) or thymalfasin (thymosin alpha 1; SciClone/Sigma-Tau). The anti-inflammatory drug can be, for example, CTS-1027 (Conatus) or JBK-122 (Jenken Biosciences). The thiazolides can be, for example, Alinia (nitazoxanide; Romark Laboratories). The broad spectrum immune stimulator can be, for example, SCV-07 (SciClone). The inflammatory/fibrosis inhibitor can be, for example, MitoQ (mitoquinone; Antipodean Pharmaceuticals). The cyclophilin inhibitor can be, for example, DEBIO-025 (Debio Pharm Group). The pancaspase inhibitor can be, for example, PF-03491390 (formerly IDN-6556; Pfizer Pharmaceuticals). The HCV immune globulin can be, for example, Civacir (Nabi). The antiviral can be, for example, Suvus (Methylene blue, formerly BIVN-104 (Virostat); Bioenvision). Optionally, the anti-infective is Nitazoxanide (Alinia®, Romark Pharmaceuticals). The glucosidase I inhibitor can be, for example, MX-3253 (celgosivir; Migenix). The IRES inhibitor can be, for example, VGX-410C (Mifepristone; VGX Pharmaceuticals). The bezafibrate can be, for example, Hepaconda (Giaconda). The nucleoside analog can be, for example, ribavirin (Roches's Copegus or Schering-Plough's Rebetol) or viramidine (taribavirin (ribavirin pro-drug); Valeant Pharmaceuticals). Optionally, the ribavirin or viramidine is administered orally once or twice daily to the patient at a dose of about 800-1200 mg. The Type I Interferon can be, for example, Interferon alpha or pegylated Interferon alpha. Optionally, the Interferon alpha or pegylated Interferon alpha is PEGASYS (pegylated interferon-alpha-2a or peg-IFN-α-2a; Roche), PEG-INTRON (pegylated interferon-alpha-2b or peg-IFN-α-2b; Schering-Plough), Belerofon (Nautilus Biotech), oral interferon alpha (Amarillo Biosciences), BLX-883 (Locteron; Biolex Therapeutics/OctoPlus), Multiferon (Viragen), Albuferon (Human Genome Sciences), Consensus Interferon or (Infergen; Three Rivers Pharma). The Type I Interferon can be, for example, omega interferon (Intarcia Therapeutics). Optionally, the Type II Interferon is Interferon gamma, e.g., Actimmune® by Intermune. The polyethylene glycol (PEG) of the pegylated Type III Interferon can be, for example, 20 kD, 30 kD or 40 kD mPEG-propionaldehyde. The 20 kD, 30 kD or 40 kD mPEG-propionaldehyde can be conjugated, for example, to the N-terminus of the Type III Interferon polypeptide.

The present invention also provides for a method of treating a human patient infected or at risk of infection with the hepatitis C virus comprising subcutaneously administering to the human patient about 1.5-5.0 μg/kg of a pegylated polypeptide, wherein the polypeptide comprises amino acid residues 1-176 of SEQ ID NO:106, and wherein the polyethylene glycol moiety is mPEG propionaldehyde. Optionally, the mPEG propionaldehyde has a molecular weight of about 20 kD, 30 kD or 40 kD. Optionally, the mPEG propionaldehyde is linear. Optionally, the method further comprises administering a nucleoside analog before, concurrently or after administration of the pegylated polypeptide. Optionally, the patient is selected from a subpopulation of hepatitis C patients consisting of treatment naïve patients with genotype I hepatitis C; treatment naïve patients with any genotype hepatitis C (e.g., 1a, 1b, 1c, 2a, 2b, 2c, 3a, 3b, 4a, 4b, 4c, 4d, 4e, 5a, 6a, 7a, 7b, 8a, 8b, 9a, 10a, and 11a); patients co-infected with the human immunodeficiency virus (HIV); patients intolerant to Pegylated Interferon Alpha, Interferon Alpha or any other Pegylated or NonPegylated Type I Interferon; patients for whom treatment with Pegylated Interferon Alpha, Interferon Alpha or any other Pegylated or NonPegylated Type I Interferon is contraindicated; patients awaiting or following liver transplant; patients with decompensated liver disease; patients who are previous non-responders to treatment with Pegylated Interferon Alpha, Interferon Alpha or any other Pegylated or NonPegylated Type I Interferon either as a single agent or in combination with ribavirin or any other anti-hepatitis C agent, including patients who were null responders, responder/relapsers, or break-through patients; patients who were non-compliant with prior treatment with Pegylated Interferon Alpha, Interferon Alpha or any other Pegylated or NonPegylated Type I Interferon either as a single agent or in combination with ribavirin or other any of the anti-hepatitis C agents; patients with any base level of hepatitis C RNA; and patients with cirrhosis. Optionally, the duration of the treatment is less than 20 weeks, 20 weeks, 24 weeks, 28 weeks, 32 weeks, 36 weeks, 40 weeks, 44 weeks, 48 weeks, 52 weeks or greater than 52 weeks.

The present invention also provides for a method of treating a human patient infected or at risk of infection with the hepatitis C virus comprising subcutaneously administering to the human patient a pharmaceutical formulation comprising about 1.5-5.0 μg/kg of a pegylated polypeptide and a pharmaceutically acceptable vehicle, wherein the polypeptide comprises amino acid residues 1-176 of SEQ ID NO:106, and wherein the pegylated polypeptide is pegylated with mPEG propionaldehyde. Optionally, the mPEG propionaldehyde has a molecular weight of about 20 kD, 30 kD or 40 kD. Optionally, the mPEG propionaldehyde is linear. Optionally, the method further comprises administering a nucleoside analog before, concurrently or after administration of the pegylated polypeptide. Optionally, the patient is selected from a subpopulation of hepatitis C patients consisting of treatment naïve patients with genotype I hepatitis C; treatment naïve patients with any genotype hepatitis C; patients co-infected with the human immunodeficiency virus (HIV); patients intolerant to Pegylated Interferon Alpha, Interferon Alpha or any other Pegylated or NonPegylated Type I Interferon; patients for whom treatment with Pegylated Interferon Alpha, Interferon Alpha or any other Pegylated or NonPegylated Type I Interferon is contraindicated; patients awaiting or following liver transplant; patients with decompensated liver disease; patients who are previous non-responders to treatment with Pegylated Interferon Alpha, Interferon Alpha or any other Pegylated or NonPegylated Type I Interferon either as a single agent or in combination with ribavirin or any other anti-hepatitis C agent, including patients who were null responders, responder/relapsers, or break-through patients; patients who were non-compliant with prior treatment with Pegylated Interferon Alpha, Interferon Alpha or any other Pegylated or NonPegylated Type I Interferon either as a single agent or in combination with ribavirin or other any of the anti-hepatitis C agents; patients with any base level of hepatitis C RNA; and patients with cirrhosis. Optionally, the duration of the treatment is less than 20 weeks, 20 weeks, 24 weeks, 28 weeks, 32 weeks, 36 weeks, 40 weeks, 44 weeks, 48 weeks, 52 weeks or greater than 52 weeks.

The present invention also provides a method of treating a responder/relapser human patient infected with the hepatitis C virus comprising subcutaneously administering to the human patient about 1.5-5.0 μg/kg of a pegylated polypeptide, wherein the polypeptide comprises amino acid residues 1-176 of SEQ ID NO:106, and wherein the pegylated polypeptide is pegylated with mPEG propionaldehyde having a molecular weight of about 20 kD. Optionally, the duration of the treatment is less than 20 weeks, 20 weeks, 24 weeks, 28 weeks, 32 weeks, 36 weeks, 40 weeks, 44 weeks, 48 weeks, 52 weeks or greater than 52 weeks.

The present invention also provides a method of treating a responder/relapser human patient infected with the hepatitis C virus comprising subcutaneously administering to the human patient a pharmaceutical formulation comprising about 1.5-5.0 μg/kg of a pegylated polypeptide and a pharmaceutically acceptable vehicle, wherein the polypeptide comprises amino acid residues 1-176 of SEQ ID NO:106, and wherein the pegylated polypeptide is pegylated with a polyethylene glycol moiety. Optionally, the polyethylene glycol moiety is mPEG propionaldehyde with a molecular weight of about 20 kD. Optionally, the duration of the treatment is less than 20 weeks, 20 weeks, 24 weeks, 28 weeks, 32 weeks, 36 weeks, 40 weeks, 44 weeks, 48 weeks, 52 weeks or greater than 52 weeks.

The present invention also provides for a method of treating a treatment naïve human patient infected or at risk of infection with the hepatitis C virus comprising subcutaneously administering to the human patient a pharmaceutical formulation comprising about 1.5-5.0 μg/kg of a pegylated polypeptide and a pharmaceutically acceptable vehicle, wherein the polypeptide comprises amino acid residues 1-176 of SEQ ID NO:106, and wherein the pegylated polypeptide is pegylated with mPEG propionaldehyde. Optionally, the mPEG propionaldehyde has a molecular weight of about 20 kD, 30 kD or 40 kD. Optionally, the mPEG propionaldehyde is linear. Optionally, the method further comprises administering a nucleoside analog before, concurrently or after administration of the pharmaceutical formulation.

IV. Articles of Manufacture

In another embodiment of the invention, an article of manufacture containing materials useful for the treatment of hepatitis C as described above is provided. The article of manufacture comprises a vial with a fixed dose of the Pegylated Type III Interferon contained therein and, optionally, a package insert. The vial may be formed from a variety of materials such as glass or plastic, and may be sealed by a stopper pierceable by a syringe. For example, the vial may be a formal vitrum type I glass vial with a dose as described herein, with DAIKYO GREY™ fluoro-resin laminated stopper, and 20 mm flip top aluminum cap. The article of manufacture may further include other materials desirable from a commercial and user standpoint, including other buffers, diluents, filters, needles, and syringes, etc.

The article of manufacture preferably further comprises a package insert. The package insert may provide instructions to administer the dose to a hepatitis C patient.

The following examples are offered to further illustrate the various specific and preferred embodiments and techniques. It should be understood, however, that many variations and modifications may be made while remaining within the scope of the present invention, so the scope of the invention is not intended to be limited by the examples.

### EXAMPLES

### Example 1

### Human Clinical Trial Studying Peg-rIL-29 in Patients or Subjects with Chronic Genotype 1 Hepatitis C Virus Infection Who have Relapsed Following Prior Treatment with a Pegylated IFN-α and Ribavirin

A 3-part, Phase 1b, dose- and schedule-escalation study of PEG-rIL-29 (SEQ ID NO:106 conjugated to a 20 kD mPEG-propionaldehyde, which is produced and purified as described in WO 07/041,713, was the pegylated polypeptide used in this Example 1) administered subcutaneously (SC) as a single agent and in combination with ribavirin (RBV) in subjects with chronic hepatitis C genotype 1 virus infection who have relapsed following interferon-alpha-based treatment (Parts 1 and 2) or who are naïve to treatment (Part 3) was performed. Part 1 of the study evaluated escalating doses of single agent PEG-rIL-29 given either once every two weeks (Q2W) or weekly (QW) for 4 weeks. Parts 2 and 3 of this study evaluated escalating doses of PEG-rIL-29 administered weekly in combination with daily ribavirin for 4 weeks. Study assessments include HCV RNA levels, documentation of adverse events and various laboratory measurements. Samples to detect the presence of anti-PEG-rIL-29 antibodies were collected through Day 59. Pharmacokinetic assessments include serum levels of PEG-rIL-29.

PEG-rIL-29 dosing and study assessment days are presented in Table 4.

TABLE 4
Timing of PEG-rIL-29 administration and evaluations

| | Study Day: | | | | | | |
|---|---|---|---|---|---|---|---|
| Schedule | 1 | 8 | 15 | 22 | 29 | 36 | 59 |
| Q2W | | | | | | | |
| PEG-rIL-29 Administration | X | | X | | | | |
| Evaluations | X¹ | X | X¹ | X | X | | X |
| QW | | | | | | | |
| PEG-rIL-29 Administration | X | X | X | X | | | |
| Evaluations | X¹ | X¹ | X¹ | X¹ | X | X | X |

Q2W = every 2 weeks;
QW = weekly
Pre-dose

Each cohort consists of 6 evaluable subjects. To be considered evaluable, a subject must have completed all study visits through Day 29 (every 2 weeks cohorts) or Day 36 (weekly cohorts) unless the reason for not doing so is due to PEG-rIL-29-related toxicity. A dose level or schedule is considered not tolerated if 2 or more subjects experience dose-limiting toxicity (DLT), or 2 or more subjects are unable to receive all planned doses due to treatment-related toxicity.

Details of cohorts evaluated, in addition to those currently open to enrollment are provided in Table 5.

TABLE 5
PEG-rIL-29 dose level and schedules evaluated to date

| | | No. of Subjects |
|---|---|---|
| | Dose Level | Treated |
| Part 1 | 1.5 μg/kg Q2W (Cohort 1) | 6 |
| | 3.0 μg/kg Q2W (Cohort 2) | 6 |
| | 1.5 μg/kg QW (Cohort 3) | 6 |
| | 3.0 μg/kg QW (Cohort 4) | 6 |
| Part 2 | 0.5 μg/kg QW + daily RBV (Cohort 7) | 4, enrolling |
| | 0.75 μg/kg QW + daily RBV (Cohort 6) | 3, enrolling |
| | 1.5 μg/kg QW + daily RBV (Cohort 5) | 7¹ |
| | 2.25 μg/kg QW + daily RBV (Cohort 8) | 4, enrolling |
| Part 3 | 1.5 μg/kg QW + daily RBV (Cohort 9) | 2, enrolling |

Q2W = every 2 weeks; QW = weekly; RVB = ribavirin
One subject who experienced an unrelated SAE necessitating discontinuation of study drug after Day 8 was replaced.

Subject demographics and baseline characteristics are summarized in Tables 7 and 8.

Antiviral Activity

Antiviral activity, defined as a >1-log decrease in HCV RNA from baseline any time on study, has been observed at all dose levels studied to date. As illustrated in Table 6 weekly dosing is associated with greater and more consistent decreases in HCV RNA than every 2 weeks dosing, with a mean maximum decrease >3 log from baseline for all cohorts treated weekly regardless of dose level or combination with ribavirin. Three subjects (Subjects 502-0065, 502-0070 and 507-0071) treated in the 3.0 μg/kg weekly cohort did achieve undetectable HCV RNA levels prior to Day 29. Baseline viral loads for these subjects (502-0065, 507-0071 and 502-0070) were 16,400, 213,000, and 1,000,000 IU/mL, respectively.

TABLE 6
Maximum viral load reduction from baseline by cohort

| | | | QW | | | | |
|---|---|---|---|---|---|---|---|
| | Q2W | | | | 0.5 μg/kg + | 0.75 μg/kg + | 1.5 μg/kg + |
| | 1.5 μg/kg | 3.0 μg/kg | 1.5 μg/kg | 3.0 μg/kg | RBV | RBV | RBV |
| Status | (N = 6) | (N = 6) | (N = 6) | (N = 6) | (N = 3) | (N = 3) | (N = 6) |
| n | 6 | 6 | 6 | 6 | 3 | 3 | 6 |
| Mean Log | 2.2 | 1.9 | 3.6 | 3.4 | 3.0 | 3.0 | 3.2 |
| Decrease | | | | | | | |
| Range | 0.6-5.2 | 1.0-3.0 | 2.0-5.0 | 2.5-4.6 | 0.7-3.4 | 1.7-4.7 | 0.1-5.6 |

Q2W = every 2 weeks;
QW = weekly;
RBV = ribavirin
HCV RNA levels evalulated by reverse transcriptase polymerase chain reaction (RT-PCR) based assay

### Results

TABLE 7
Demographics and Subject Characteristics

| | | | Date of Birth | | | | Height | Weight | BMI |
|---|---|---|---|---|---|---|---|---|---|
| Subject | Treatment | Cohort | (Y-M-D) | Age (yr) | Gender | Race | (cm) | (kg) | (kg/m2) |
| 502-0001 | 1.5 μg/kg Q2W | 1 | 1952-02-25 | 55 | F | HISPANIC | 161.3 | 64.8 | 24.9 |
| 502-0003 | 1.5 μg/kg Q2W | 1 | 1947-06-20 | 60 | M | HISPANIC | 177.8 | 92.7 | 29.3 |
| 502-0008 | 1.5 μg/kg Q2W | 1 | 1961-03-02 | 47 | M | HISPANIC | 180.3 | 89.2 | 27.4 |
| 502-0009 | 1.5 μg/kg Q2W | 1 | 1949-04-03 | 58 | F | HISPANIC | 160 | 71.4 | 27.9 |
| 502-0012 | 1.5 μg/kg Q2W | 1 | 1960-07-22 | 47 | M | HISPANIC | 172.7 | 89.1 | 29.9 |
| 505-0006 | 1.5 μg/kg Q2W | 1 | 1958-01-05 | 50 | M | WHITE | 188 | 107.3 | 30.4 |
| 501-0015 | 3.0 μg/kg Q2W | 2 | 1955-12-20 | 52 | F | BLACK OR | 156.2 | 118.7 | 48.7 |
| | | | | | | AFRICAN AMERICAN | | | |
| 501-0017 | 3.0 μg/kg Q2W | 2 | 1958-07-06 | 49 | F | WHITE | 166.4 | 87.1 | 31.5 |
| 501-0021 | 3.0 μg/kg Q2W | 2 | 1959-05-30 | 48 | F | WHITE | 172.7 | 83.9 | 28.1 |
| 502-0013 | 3.0 μg/kg Q2W | 2 | 1957-03-04 | 51 | M | HISPANIC | 175.3 | 77.3 | 25.2 |
| 502-0019 | 3.0 μg/kg Q2W | 2 | 1964-10-29 | 43 | F | WHITE | 167.6 | 100.9 | 35.9 |
| 502-0020 | 3.0 μg/kg Q2W | 2 | 1957-06-18 | 50 | F | WHITE | 165.1 | 75.5 | 27.7 |
| 502-0023 | 1.5 μg/kg QW | 3 | 1960-01-11 | 48 | F | HISPANIC | 160 | 61.8 | 24.1 |
| 502-0024 | 1.5 μg/kg QW | 3 | 1941-10-16 | 66 | M | HISPANIC | 165.1 | 75.9 | 27.8 |
| 503-0022 | 1.5 μg/kg QW | 3 | 1957-12-21 | 50 | M | WHITE | 179.9 | 128.3 | 39.6 |
| 505-0027 | 1.5 μg/kg QW | 3 | 1950-04-02 | 58 | F | HISPANIC | 160 | 78 | 30.5 |
| 506-0032 | 1.5 μg/kg QW | 3 | 1955-04-27 | 53 | M | WHITE | 163.3 | 107 | 40.1 |
| 507-0028 | 1.5 μg/kg QW | 3 | 1945-11-24 | 62 | M | BLACK OR | 167.6 | 67.7 | 24.1 |
| | | | | | | AFRICAN AMERICAN | | | |

TABLE 8
Demographics and Subject Characteristics
Subjects with Weekly Combination Therapy (PEG-rIL-29 + Ribavirin)

| | | | | | | 1.5 μg/kg | |
|---|---|---|---|---|---|---|---|
| | | 0.5 μg/kg | 0.75 μg/kg | 1.5 μg/kg | 2.25 μg/kg | (Naive) | Total |
| Parameter | Category/Statistic | (N = 4) | (N = 3) | (N = 7) | (N = 4) | (N = 2) | (N = 20) |
| Age (years) | n | 4 | 3 | 7 | 4 | 2 | 20 |
| | Mean (SD) | 55.5 (4.0) | 50.7 (5.9) | 52.9 (7.9) | 52.8 (3.5) | 47.5 (2.1) | 52.5 (5.8) |
| | Median | 56.5 | 53.0 | 57.0 | 52.5 | 47.5 | 53.5 |
| | Min, Max | 50, 59 | 44, 55 | 36, 59 | 49, 57 | 46, 49 | 36, 59 |
| Gender, n (%) | F | 0 | 0 | 2 (29) | 1 (25) | 0 | 3 (15) |
| | M | 4 (100) | 3 (100) | 5 (71) | 3 (75) | 2 (100) | 17 (85) |
| Race, n (%) | Black Or African | 2 (50) | 0 | 1 (14) | 1 (25) | 1 (50) | 5 (25) |
| | American | | | | | | |
| | Hispanic | 0 | 2 (67) | 0 | 0 | 1 (50) | 3 (15) |
| | White | 2 (50) | 1 (33) | 6 (86) | 3 (75) | 0 | 12 (60) |
| Height (cm) | n | 4 | 3 | 7 | 4 | 2 | 20 |
| | Mean (SD) | 178.40 (1.63) | 176.93 (3.87) | 169.84 (12.87) | 177.88 (9.52) | 174.00 (1.84) | 174.64 (9.13) |
| | Median | 178.40 | 177.80 | 170.20 | 179.20 | 174.00 | 177.80 |
| | Min, Max | 176.5, 180.3 | 172.7, 180.3 | 150.0, 182.9 | 165.1, 188.0 | 172.7, 175.3 | 150.0, 188.0 |
| Weight (kg) | n | 4 | 3 | 7 | 4 | 2 | 20 |
| | Mean (SD) | 104.83 (19.74) | 95.63 (12.00) | 88.86 (9.81) | 103.50 (27.44) | 96.90 (18.95) | 96.80 (17.07) |
| | Median | 111.40 | 95.70 | 89.00 | 107.25 | 96.90 | 93.55 |
| | Min, Max | 76.5, 120.0 | 83.6, 107.6 | 77.3, 106.0 | 69.5, 130.0 | 83.5, 110.3 | 69.5, 130.0 |
| BMI (kg/m²) | n | 4 | 3 | 7 | 4 | 2 | 20 |
| | Mean (SD) | 32.98 (6.63) | 30.60 (4.35) | 31.00 (3.95) | 32.58 (7.22) | 31.95 (5.59) | 31.75 (4.96) |
| | Median | 34.95 | 32.10 | 30.20 | 35.60 | 31.95 | 31.90 |
| | Min, Max | 23.5, 38.5 | 25.7, 34.0 | 26.6, 39.1 | 21.9, 37.2 | 28.0, 35.9 | 21.9, 39.1 |

a Rbv = Ribavirin

TABLE 9
Disease/Treatment History

| | | | | | | | | Viral | Viral |
|---|---|---|---|---|---|---|---|---|---|
| | | | Disease | | | Treatment | | Clearance | Clearance |
| | | Diagnosis | Duration | | Treatment | Duration | Viral | Start-End | Duration |
| Subject | Treatment | Date | (Yrs) | Treatment (Form) | Start-End | (Weeks) | Clearance? | Dates | (Wks) |
| 501- | 3.0 μg/kg | 2005 | 2.8 | PEG-IFN-ALPHA/ | 2006-06-16-2007- | 47.1 | Y | 2006-09- | 41.1 |
| 0015 | Q2W | | | RIBAVIRIN | 05-11 | | | 08-2007- | |
| | | | | (PEGASYS) | | | | 06- | |
| | | | | | | | | 22 | |
| 501- | 3.0 μg/kg | 2005-09- | 2.6 | PEG-IFN-ALPHA/ | 2005-09-28-2006- | 46 | Y | 2006-03- | 65 |
| 0017 | Q2W | 08 | | RIBAVIRIN | 08 | | | 01-2007- | |
| | | | | (PEGINTRON) | | | | 05- | |
| | | | | | | | | 29 | |
| 501- | 3.0 μg/kg | 2004 | 3.9 | PEG-IFN-ALPHA/ | 2004-11-2005- | 52.3 | Y | 2005-05- | 28.1 |
| 0021 | Q2W | | | RIBAVIRIN | 11 | | | 26-2005- | |
| | | | | (PEGASYS) | | | | 12- | |
| | | | | | | | | 08 | |
| 502- | 1.5 μg/kg | 1997 | 10.6 | PEG-IFN-ALPHA/ | 2001-11-2002- | 52.3 | Y | 2002-02- | 53.1 |
| 0001 | Q2W | | | RIBAVIRIN | 11 | | | 06-2003- | |
| | | | | (PEGINTRON) | | | | 02- | |
| | | | | | | | | 12 | |
| 502- | 1.5 μg/kg | 2002-01 | 6 | PEG-IFN-ALPHA/ | 2002-06-2003- | 47.9 | Y | 2002-10- | 24.1 |
| 0003 | Q2W | | | RIBAVIRIN | 05 | | | 14-2003- | |
| | | | | (PEGINTRON) | | | | 03- | |
| | | | | | | | | 31 | |
| 502- | 1.5 μg/kg | | | INTERFERON + | 2003-05-2004- | 48.1 | N | | |
| 0003 | Q2W | | | RIBAVIRIN | 04 | | | | |
| 502- | 1.5 μg/kg | | | ACTILON | 2005-2005 | 0.1 | N | | |
| 0003 | Q2W | | | | | | | | |
| 502- | 1.5 μg/kg | 2004 | 3.7 | PEG-IFN-ALPHA/ | 2005-01-2005- | 47.9 | Y | 2005-04- | 34.7 |
| 0008 | Q2W | | | RIBAVIRIN | 12 | | | 22-2005- | |
| | | | | (PEGASYS) | | | | 12- | |
| | | | | | | | | 20 | |
| 502- | 1.5 μg/kg | 2000 | 7.7 | PEG-IFN-ALPHA/ | 2003-06-18-2004- | 45.3 | Y | 2003-09- | 49 |
| 0009 | Q2W | | | RIBAVIRIN | 04-29 | | | 11-2004- | |
| | | | | (PEGASYS) | | | | 08- | |
| | | | | | | | | 18 | |
| 502- | 1.5 μg/kg | | | PEGYLATED | 2001-07-12-2002- | 49 | Y | UNK- | |
| 0009 | Q2W | | | INTERFERON WITH | 06-19 | | | UNK | |
| | | | | RIBAVIRIN | | | | | |
| 502- | 1.5 μg/kg | 2001 | 6.8 | PEG-IFN-ALPHA/ | 2002-02-24-2003- | 48.1 | Y | 2002-12- | 9.6 |
| 0012 | Q2W | | | RIBAVIRIN | 01-26 | | | 03-2003- | |
| | | | | (PEGINTRON) | | | | 02- | |
| | | | | | | | | 07 | |
| 502- | 1.5 μg/kg | | | PEG-INTERFERON | 2007-09-13-2008- | 16.3 | N | | |
| 0012 | Q2W | | | AND RIBAVIRIN | 01-04 | | | | |
| 502- | 3.0 μg/kg | 2000 | 7.8 | PEG-IFN-ALPHA/ | 2003-08-01-2004- | 30.6 | Y | 2003-10- | 27.4 |
| 0013 | Q2W | | | RIBAVIRIN | 03-01 | | | 20-2004- | |
| | | | | (PEGASYS) | | | | 04- | |
| | | | | | | | | 28 | |
| 502- | 3.0 μg/kg | 2005-09 | 2.6 | PEG-IFN-ALPHA/ | 2005-09-26-2006- | 45.3 | Y | 2006-01- | 41.1 |
| 0019 | Q2W | | | RIBAVIRIN | 08-08 | | | 17-2006- | |
| | | | | (PEGASYS) | | | | 10- | |
| | | | | | | | | 31 | |
| 502- | 3.0 μg/kg | | | PROTEASE | 2005-12-21-2006- | 4 | Y | 2006-01- | 41.1 |
| 0019 | Q2W | | | INHIBITOR | 01-17 | | | 17-2006- | |
| | | | | | | | | 10- | |
| | | | | | | | | 31 | |
| 502- | 3.0 μg/kg | 2006-08 | 1.7 | PEG-IFN-ALPHA/ | 2006-09-08-2007- | 62 | Y | 2007-02- | 45.1 |
| 0020 | Q2W | | | RIBAVIRIN | 11 | | | 09-2007- | |
| | | | | (PEGINTRON) | | | | 12- | |
| | | | | | | | | 21 | |
| 502- | 1.5 μg/kg | 2003 | 4.9 | PEG-IFN-ALPHA/ | 2004-02-03-2005- | 49.7 | Y | 2004-10- | 38.4 |
| 0023 | QW | | | RIBAVIRIN | 01 | | | 11-2005- | |
| | | | | (PEGINTRON) | | | | 07- | |
| | | | | | | | | 06 | |
| 502- | 1.5 μg/kg | 1999 | 9 | PEG-IFN-ALPHA/ | 2006-11-01-2007- | 19.3 | Y | 2007-01- | 19.4 |
| 0024 | QW | | | RIBAVIRIN | 03 | | | 09-2007- | |
| | | | | (PEGINTRON) | | | | 05- | |
| | | | | | | | | 24 | |
| 503- | 1.5 μg/kg | 1999 | 8.9 | PEG-IFN-ALPHA/ | | | | | |
| 0022 | QW | | | RIBAVIRIN | | | | | |
| 503- | 1.5 μg/kg | | | CONSENSUS IFN | 2005-02-06-2006- | 49.1 | Y | 2006-01- | 14.1 |
| 0022 | QW | | | | 01 | | | 09-2006- | |
| | | | | | | | | 04- | |
| | | | | | | | | 17 | |
| 505- | 1.5 μg/kg | 1981 | 26.7 | PEG-IFN-ALPHA/ | 2004-08-31-2005- | 47.6 | Y | 2004-10- | 70.4 |
| 0006 | Q2W | | | RIBAVIRIN | 07-29 | | | 26-2006- | |
| | | | | (PEGASYS) | | | | 03- | |
| | | | | | | | | 02 | |
| 505- | 1.5 μg/kg | 2003 | 5 | PEG-IFN-ALPHA/ | 2006-05-18-2007- | 47.7 | Y | 2006-06- | 56.1 |
| 0027 | QW | | | RIBAVIRIN | 04-16 | | | 15- | |
| | | | | (PEGASYS) | | | | 2007-07- | |
| | | | | | | | | 12 | |
| 506- | 1.5 μg/kg | 1995 | 13 | PEG-IFN-ALPHA/ | 1996-01-1996- | 47.7 | Y | 1996-09- | 23.4 |
| 0032 | QW | | | RIBAVIRIN | 12-13 | | | 11- | |
| | | | | (PEGINTRON) | | | | 1997-02- | |
| | | | | | | | | 21 | |
| 506- | 1.5 μg/kg | | | CONSENSUS | 2003-2004 | 52.4 | Y | | |
| 0032 | QW | | | IFN/RIBA | | | | | |
| 507- | 1.5 μg/kg | 2006-01- | 2.5 | PEG-IFN-ALPHA/ | - | | Y | 2007-10- | 27.1 |
| 0028 | QW | 01 | | RIBAVIRIN | 2007-10-17 | | | 15- | |
| | | | | | | | | 2008-04- | |
| | | | | | | | | 21 | |
| 507- | 1.5 μg/kg | | | ALBUFERON | 2007-06-28-2007- | 16 | Y | 2007-10- | 27.1 |
| 0028 | QW | | | INTERFERON | 10-17 | | | 15- | |
| | | | | 900MCG | | | | 2008-04- | |
| | | | | | | | | 21 | |

TABLE 10
Descriptive Statistics for HCV RNA

| | Category/ | 1.5 μg/kg Q2W | 3.0 μg/kg Q2W | 1.5 μg/kg QW | Total |
|---|---|---|---|---|---|
| | Statistic | (N = 6) | (N = 6) | (N = 6) | (N = 18) |
| Parameter | n | 6 | 6 | 6 | 18 |
| Baseline HCV (IU/mL) | Mean (SD) | 23566666.7 | 4288333.3 | 7957666.7 | 11937555.6 |
| | | (9346585.8) | (5135186.1) | (9455885.8) | (11563713.4) |
| | Median | 21350000.0 | 2490000.0 | 3355000.0 | 8460000.0 |
| | Min, Max | 12000000, | 1300000, | 856000, | 856000, |
| | | 36100000 | 14700000 | 23800000 | 36100000 |
| Baseline HCV (log scale) | Mean (SD) | 7.343 (0.180) | 6.465 (0.369) | 6.583 (0.601) | 6.797 (0.562) |
| | Median | 7.330 | 6.395 | 6.470 | 6.885 |
| | Min, Max | 7.08, 7.56 | 6.11, 7.17 | 5.93, 7.38 | 5.93, 7.56 |
| Maximum decrease from | Mean (SD) | 2.152 (1.647) | 1.893 (0.888) | 3.600 (1.270) | 2.548 (1.449) |
| baseline (log scale) | Median | 1.925 | 1.810 | 3.885 | 2.305 |
| | Min, Max | 0.59, 5.18 | 0.98, 3.01 | 2.05, 4.95 | 0.59, 5.18 |

TABLE 11
HCV RNA Level

| | | | | | HCV |
|---|---|---|---|---|---|
| | | | | HCV RNA | Change from |
| | | | Study | Level - | Baseline - |
| Subject | Treatment | Visit | Day | Log Scale | Log Scale |
| 502-0001 | 1.5 μg/kg Q2W | Day 1 | 1 | 7.56 | |
| 502-0001 | 1.5 μg/kg Q2W | Day 2 | 2 | 6.78 | 0.78 |
| 502-0001 | 1.5 μg/kg Q2W | Day 4 | 4 | 5.10 | 2.46 |
| 502-0001 | 1.5 μg/kg Q2W | Day 8 | 8 | 5.82 | 1.74 |
| 502-0001 | 1.5 μg/kg Q2W | Day 15 | 15 | 6.87 | 0.69 |
| 502-0001 | 1.5 μg/kg Q2W | Day 22 | 22 | 5.86 | 1.70 |
| 502-0001 | 1.5 μg/kg Q2W | Day 29 | 29 | 6.91 | 0.65 |
| 502-0001 | 1.5 μg/kg Q2W | Day 59 | 59 | 7.70 | −0.14 |
| 502-0003 | 1.5 μg/kg Q2W | Day 1 | 1 | 7.37 | |
| 502-0003 | 1.5 μg/kg Q2W | Day 2 | 2 | 7.21 | 0.16 |
| 502-0003 | 1.5 μg/kg Q2W | Day 4 | 4 | 6.78 | 0.59 |
| 502-0003 | 1.5 μg/kg Q2W | Day 8 | 8 | 7.16 | 0.21 |
| 502-0003 | 1.5 μg/kg Q2W | Day 15 | 15 | 7.00 | 0.37 |
| 502-0003 | 1.5 μg/kg Q2W | Day 22 | 22 | 7.13 | 0.24 |
| 502-0003 | 1.5 μg/kg Q2W | Day 29 | 29 | 7.00 | 0.37 |
| 502-0003 | 1.5 μg/kg Q2W | Day 59 | 59 | 7.01 | 0.36 |
| 502-0008 | 1.5 μg/kg Q2W | Day 1 | 1 | 7.08 | |
| 502-0008 | 1.5 μg/kg Q2W | Day 2 | 2 | 5.96 | 1.12 |
| 502-0008 | 1.5 μg/kg Q2W | Day 4 | 4 | 5.18 | 1.90 |
| 502-0008 | 1.5 μg/kg Q2W | Day 8 | 8 | 6.22 | 0.86 |
| 502-0008 | 1.5 μg/kg Q2W | Day 15 | 15 | 6.45 | 0.63 |
| 502-0008 | 1.5 μg/kg Q2W | Day 22 | 22 | 5.78 | 1.30 |
| 502-0008 | 1.5 μg/kg Q2W | Day 29 | 29 | 6.41 | 0.67 |
| 502-0008 | 1.5 μg/kg Q2W | Day 59 | 59 | 6.63 | 0.45 |
| 502-0009 | 1.5 μg/kg Q2W | Day 1 | 1 | 7.52 | |
| 502-0009 | 1.5 μg/kg Q2W | Day 2 | 2 | 7.14 | 0.38 |
| 502-0009 | 1.5 μg/kg Q2W | Day 4 | 4 | 5.57 | 1.95 |
| 502-0009 | 1.5 μg/kg Q2W | Day 8 | 8 | 6.57 | 0.95 |
| 502-0009 | 1.5 μg/kg Q2W | Day 15 | 16 | 7.26 | 0.26 |
| 502-0009 | 1.5 μg/kg Q2W | Day 22 | 22 | 6.28 | 1.24 |
| 502-0009 | 1.5 μg/kg Q2W | Day 29 | 29 | 7.17 | 0.35 |
| 502-0009 | 1.5 μg/kg Q2W | Day 59 | 59 | 7.24 | 0.28 |
| 502-0012 | 1.5 μg/kg Q2W | Day 1 | 1 | 7.24 | |
| 502-0012 | 1.5 μg/kg Q2W | Day 2 | 2 | 6.41 | 0.83 |
| 502-0012 | 1.5 μg/kg Q2W | Day 4 | 4 | 6.52 | 0.72 |
| 502-0012 | 1.5 μg/kg Q2W | Day 8 | 8 | 6.58 | 0.66 |
| 502-0012 | 1.5 μg/kg Q2W | Day 15 | 15 | 6.99 | 0.25 |
| 502-0012 | 1.5 μg/kg Q2W | Day 22 | 22 | 6.48 | 0.76 |
| 502-0012 | 1.5 μg/kg Q2W | Day 29 | 29 | 6.95 | 0.29 |
| 502-0012 | 1.5 μg/kg Q2W | Day 59 | 59 | 6.75 | 0.49 |
| 505-0006 | 1.5 μg/kg Q2W | Day 1 | 1 | 7.29 | |
| 505-0006 | 1.5 μg/kg Q2W | Day 2 | 2 | 5.49 | 1.80 |
| 505-0006 | 1.5 μg/kg Q2W | Day 4 | 4 | 3.58 | 3.71 |
| 505-0006 | 1.5 μg/kg Q2W | Day 8 | 8 | 4.56 | 2.73 |
| 505-0006 | 1.5 μg/kg Q2W | Day 15 | 16 | 3.57 | 3.72 |
| 505-0006 | 1.5 μg/kg Q2W | Day 22 | 23 | 2.11 | 5.18 |
| 505-0006 | 1.5 μg/kg Q2W | Day 29 | 29 | 2.49 | 4.80 |
| 505-0006 | 1.5 μg/kg Q2W | Day 59 | 59 | 7.30 | −0.01 |
| 501-0015 | 3.0 μg/kg Q2W | Day 1 | 1 | 6.38 | |
| 501-0015 | 3.0 μg/kg Q2W | Day 2 | 2 | 5.65 | 0.73 |
| 501-0015 | 3.0 μg/kg Q2W | Day 4 | 3 | 3.98 | 2.40 |
| 501-0015 | 3.0 μg/kg Q2W | Day 8 | 8 | 4.65 | 1.73 |
| 501-0015 | 3.0 μg/kg Q2W | Day 15 | 15 | 5.15 | 1.23 |
| 501-0015 | 3.0 μg/kg Q2W | Day 22 | 22 | 3.37 | 3.01 |
| 501-0015 | 3.0 μg/kg Q2W | Day 29 | 31 | 3.81 | 2.57 |
| 501-0015 | 3.0 μg/kg Q2W | Day 59 | 66 | 5.73 | 0.65 |
| 501-0017 | 3.0 μg/kg Q2W | Day 1 | 1 | 6.41 | |
| 501-0017 | 3.0 μg/kg Q2W | Day 2 | 2 | 5.97 | 0.44 |
| 501-0017 | 3.0 μg/kg Q2W | Day 4 | 3 | 5.54 | 0.87 |
| 501-0017 | 3.0 μg/kg Q2W | Day 8 | 8 | 6.12 | 0.29 |
| 501-0017 | 3.0 μg/kg Q2W | Day 15 | 15 | 6.48 | −0.07 |
| 501-0017 | 3.0 μg/kg Q2W | Day 22 | 22 | 5.43 | 0.98 |
| 501-0017 | 3.0 μg/kg Q2W | Day 29 | 29 | 6.13 | 0.28 |
| 501-0017 | 3.0 μg/kg Q2W | Day 59 | 57 | 6.55 | −0.14 |
| 501-0021 | 3.0 μg/kg Q2W | Day 1 | 1 | 6.25 | |
| 501-0021 | 3.0 μg/kg Q2W | Day 2 | 2 | 5.65 | 0.60 |
| 501-0021 | 3.0 μg/kg Q2W | Day 4 | 3 | 5.10 | 1.15 |
| 501-0021 | 3.0 μg/kg Q2W | Day 8 | 8 | 6.07 | 0.18 |
| 501-0021 | 3.0 μg/kg Q2W | Day 15 | 15 | 6.24 | 0.01 |
| 501-0021 | 3.0 μg/kg Q2W | Day 22 | 22 | 5.91 | 0.34 |
| 501-0021 | 3.0 μg/kg Q2W | Day 29 | 31 | 6.30 | −0.05 |
| 501-0021 | 3.0 μg/kg Q2W | Day 59 | 59 | 6.45 | −0.20 |
| 502-0013 | 3.0 μg/kg Q2W | Day 1 | 1 | 7.17 | |
| 502-0013 | 3.0 μg/kg Q2W | Day 2 | 2 | 6.41 | 0.76 |
| 502-0013 | 3.0 μg/kg Q2W | Day 4 | 3 | 5.16 | 2.01 |
| 502-0013 | 3.0 μg/kg Q2W | Day 8 | 8 | 5.84 | 1.33 |
| 502-0013 | 3.0 μg/kg Q2W | Day 15 | 15 | 6.23 | 0.94 |
| 502-0013 | 3.0 μg/kg Q2W | Day 22 | 22 | 4.73 | 2.44 |
| 502-0013 | 3.0 μg/kg Q2W | Day 29 | 29 | 5.50 | 1.67 |
| 502-0013 | 3.0 μg/kg Q2W | Day 59 | 59 | 6.30 | 0.87 |
| 502-0019 | 3.0 μg/kg Q2W | Day 1 | 1 | 6.11 | |
| 502-0019 | 3.0 μg/kg Q2W | Day 2 | 2 | 5.53 | 0.58 |
| 502-0019 | 3.0 μg/kg Q2W | Day 4 | 3 | 4.93 | 1.18 |
| 502-0019 | 3.0 μg/kg Q2W | Day8 | 8 | 6.27 | −0.16 |
| 502-0019 | 3.0 μg/kg Q2W | Day 15 | 15 | | |
| 502-0019 | 3.0 μg/kg Q2W | Day 22 | 22 | 5.27 | 0.84 |
| 502-0019 | 3.0 μg/kg Q2W | Day 29 | 29 | 5.68 | 0.43 |
| 502-0019 | 3.0 μg/kg Q2W | Day 59 | 59 | 6.26 | −0.15 |
| 502-0020 | 3.0 μg/kg Q2W | Day 1 | 1 | 6.47 | |
| 502-0020 | 3.0 μg/kg Q2W | Day 2 | 2 | 6.39 | 0.08 |
| 502-0020 | 3.0 μg/kg Q2W | Day 4 | 3 | 5.28 | 1.19 |
| 502-0020 | 3.0 μg/kg Q2W | Day 8 | 8 | 5.57 | 0.90 |
| 502-0020 | 3.0 μg/kg Q2W | Day 15 | 15 | 6.48 | −0.01 |
| 502-0020 | 3.0 μg/kg Q2W | Day 22 | 22 | 3.87 | 2.60 |
| 502-0020 | 3.0 μg/kg Q2W | Day 29 | 29 | 5.53 | 0.94 |
| 502-0020 | 3.0 μg/kg Q2W | Day 59 | 59 | 6.81 | −0.34 |
| 502-0023 | 1.5 μg/kg QW | Day 1 | 1 | 6.25 | |
| 502-0023 | 1.5 μg/kg QW | Day 2 | 2 | 5.25 | 1.00 |
| 502-0023 | 1.5 μg/kg QW | Day 4 | 3 | 4.31 | 1.94 |
| 502-0023 | 1.5 μg/kg QW | Day 8 | 8 | 5.35 | 0.90 |
| 502-0023 | 1.5 μg/kg QW | Day 15 | 15 | 4.72 | 1.53 |
| 502-0023 | 1.5 μg/kg QW | Day 22 | 22 | 3.82 | 2.43 |
| 502-0023 | 1.5 μg/kg QW | Day 29 | 29 | 2.87 | 3.38 |
| 502-0023 | 1.5 μg/kg QW | Day 59 | 59 | 6.34 | −0.09 |
| 502-0024 | 1.5 μg/kg QW | Day 1 | 1 | 7.18 | |
| 502-0024 | 1.5 μg/kg QW | Day 2 | 2 | 6.28 | 0.90 |
| 502-0024 | 1.5 μg/kg QW | Day 4 | 3 | 5.52 | 1.66 |
| 502-0024 | 1.5 μg/kg QW | Day 8 | 8 | 6.16 | 1.02 |
| 502-0024 | 1.5 μg/kg QW | Day 15 | 15 | 4.85 | 2.33 |
| 502-0024 | 1.5 μg/kg QW | Day 22 | 22 | 3.79 | 3.39 |
| 502-0024 | 1.5 μg/kg QW | Day 29 | 29 | 2.79 | 4.39 |
| 502-0024 | 1.5 μg/kg QW | Day 59 | 59 | 7.30 | −0.12 |
| 503-0022 | 1.5 μg/kg QW | Day 1 | 1 | 7.38 | |
| 503-0022 | 1.5 μg/kg QW | Day 2 | 2 | 6.62 | 0.76 |
| 503-0022 | 1.5 μg/kg QW | Day 4 | 4 | 4.21 | 3.17 |
| 503-0022 | 1.5 μg/kg QW | Day 8 | 8 | 4.68 | 2.70 |
| 503-0022 | 1.5 μg/kg QW | Day 15 | 15 | 4.31 | 3.07 |
| 503-0022 | 1.5 μg/kg QW | Day 22 | 22 | 3.78 | 3.60 |
| 503-0022 | 1.5 μg/kg QW | Day 29 | 29 | 2.72 | 4.66 |
| 503-0022 | 1.5 μg/kg QW | Day 59 | 57 | 7.49 | −0.11 |
| 505-0027 | 1.5 μg/kg QW | Day 1 | 1 | 6.69 | |
| 505-0027 | 1.5 μg/kg QW | Day 2 | 2 | 5.07 | 1.62 |
| 505-0027 | 1.5 μg/kg QW | Day 4 | 3 | 4.06 | 2.63 |
| 505-0027 | 1.5 μg/kg QW | Day 8 | 8 | 4.34 | 2.35 |
| 505-0027 | 1.5 μg/kg QW | Day 15 | 15 | 2.12 | 4.57 |
| 505-0027 | 1.5 μg/kg QW | Day 22 | 22 | 2.12 | 4.57 |
| 505-0027 | 1.5 μg/kg QW | Day 29 | 29 | 1.74 | 4.95 |
| 505-0027 | 1.5 μg/kg QW | Day 59 | 59 | 6.88 | −0.19 |
| 506-0032 | 1.5 μg/kg QW | Day 1 | 1 | 6.07 | |
| 506-0032 | 1.5 μg/kg QW | Day 2 | 2 | 5.84 | 0.23 |
| 506-0032 | 1.5 μg/kg QW | Day 4 | 5 | 4.16 | 1.91 |
| 506-0032 | 1.5 μg/kg QW | Day 8 | 8 | 5.05 | 1.02 |
| 506-0032 | 1.5 μg/kg QW | Day 15 | 15 | 4.64 | 1.43 |
| 506-0032 | 1.5 μg/kg QW | Day 22 | 23 | 4.28 | 1.79 |
| 506-0032 | 1.5 μg/kg QW | Day 29 | 29 | 3.90 | 2.17 |
| 507-0028 | 1.5 μg/kg QW | Day 1 | 1 | 5.93 | |
| 507-0028 | 1.5 μg/kg QW | Day 2 | 2 | 5.99 | −0.06 |
| 507-0028 | 1.5 μg/kg QW | Day 4 | 3 | 5.32 | 0.61 |
| 507-0028 | 1.5 μg/kg QW | Day 8 | 8 | 5.37 | 0.56 |
| 507-0028 | 1.5 μg/kg QW | Day 15 | 15 | 4.90 | 1.03 |
| 507-0028 | 1.5 μg/kg QW | Day 22 | 22 | 4.25 | 1.68 |
| 507-0028 | 1.5 μg/kg QW | Day 29 | 30 | 3.88 | 2.05 |

TABLE 12
HCV RNA Level
Subjects with Weekly Combination Therapy (PEG-rIL-29 + Ribavirin)

| | | | | | | | HCV Change |
|---|---|---|---|---|---|---|---|
| | | Treatment | | Study | HCV RNA Level | HCV RNA Level - | from Baseline - |
| Subject | Cohort | (μg/kg) | Visit | Day | (IU/ml) | Log Scale | Log Scale |
| 504-0046 | 5 | 1.5 QW + RBV | Screening | −32 | 1560000 | 6.19 | |
| 504-0046 | 5 | 1.5 QW + RBV | Day 1 | 1 | 3220000 | 6.51 | |
| 504-0046 | 5 | 1.5 QW + RBV | Day 2 | 2 | 246000 | 5.39 | 1.12 |
| 504-0046 | 5 | 1.5 QW + RBV | Day 4 | 4 | 49900 | 4.70 | 1.81 |
| 504-0046 | 5 | 1.5 QW + RBV | Day 8 | 8 | 768000 | 5.89 | 0.62 |
| 504-0046 | 5 | 1.5 QW + RBV | Day 15 | 15 | 88100 | 4.94 | 1.57 |
| 504-0046 | 5 | 1.5 QW + RBV | Day 22 | 22 | 21900 | 4.34 | 2.17 |
| 504-0046 | 5 | 1.5 QW + RBV | Day 29 | 29 | 12100 | 4.08 | 2.43 |
| 504-0046 | 5 | 1.5 QW + RBV | Day 59 | 57 | 2160000 | 6.33 | 0.18 |
| 504-0053 | 5 | 1.5 QW + RBV | Screening | −8 | 1120000 | 6.05 | |
| 504-0053 | 5 | 1.5 QW + RBV | Day 1 | 1 | 1700000 | 6.23 | |
| 504-0053 | 5 | 1.5 QW + RBV | Day 2 | 2 | 262000 | 5.42 | 0.81 |
| 504-0053 | 5 | 1.5 QW + RBV | Day 4 | 3 | 119000 | 5.08 | 1.15 |
| 504-0053 | 5 | 1.5 QW + RBV | Day 8 | 9 | 132000 | 5.12 | 1.11 |
| 504-0053 | 5 | 1.5 QW + RBV | Day 15 | 15 | 1850 | 3.27 | 2.96 |
| 504-0053 | 5 | 1.5 QW + RBV | Day 22 | 22 | 933 | 2.97 | 3.26 |
| 504-0053 | 5 | 1.5 QW + RBV | Day 29 | 29 | 129 | 2.11 | 4.12 |
| 504-0053 | 5 | 1.5 QW + RBV | Day 59 | 56 | 817000 | 5.91 | 0.32 |
| 505-0050 | 5 | 1.5 QW + RBV | Screening | −22 | 17800000 | 7.25 | |
| 505-0050 | 5 | 1.5 QW + RBV | Day 1 | 1 | 9750000 | 6.99 | |
| 505-0050 | 5 | 1.5 QW + RBV | Day 2 | 2 | 792000 | 5.90 | 1.09 |
| 505-0050 | 5 | 1.5 QW + RBV | Day 4 | 3 | 51200 | 4.71 | 2.28 |
| 505-0050 | 5 | 1.5 QW + RBV | Day 8 | 8 | 255000 | 5.41 | 1.58 |
| 505-0050 | 5 | 1.5 QW + RBV | Day 15 | 15 | 30000 | 4.48 | 2.51 |
| 505-0050 | 5 | 1.5 QW + RBV | Day 22 | 22 | 5630 | 3.75 | 3.24 |
| 505-0050 | 5 | 1.5 QW + RBV | Day 29 | 30 | 1720 | 3.24 | 3.75 |
| 505-0050 | 5 | 1.5 QW + RBV | Unscheduled | 36 | 4740 | 3.68 | 3.31 |
| 505-0050 | 5 | 1.5 QW + RBV | Day 59 | 58 | 4020000 | 6.60 | 0.39 |
| 505-0057 | 5 | 1.5 QW + RBV | Unscheduled | −20 | 954000 | 5.98 | |
| 505-0057 | 5 | 1.5 QW + RBV | Screening | −10 | 1670000 | 6.22 | |
| 505-0057 | 5 | 1.5 QW + RBV | Day 1 | 1 | 774000 | 5.89 | |
| 505-0057 | 5 | 1.5 QW + RBV | Day 2 | 2 | 695000 | 5.84 | 0.05 |
| 505-0057 | 5 | 1.5 QW + RBV | Day 4 | 4 | 676000 | 5.83 | 0.06 |
| 505-0057 | 5 | 1.5 QW + RBV | Day 8 | 8 | 634000 | 5.80 | 0.09 |
| 505-0057 | 5 | 1.5 QW + RBV | Day 15 | 16 | 817000 | 5.91 | −0.02 |
| 505-0057 | 5 | 1.5 QW + RBV | Day 22 | 22 | 682000 | 5.83 | 0.06 |
| 505-0057 | 5 | 1.5 QW + RBV | Day 29 | 30 | 845000 | 5.93 | −0.04 |
| 505-0057 | 5 | 1.5 QW + RBV | Day 59 | 57 | 787000 | 5.90 | −0.01 |
| 506-0035 | 5 | 1.5 QW + RBV | Screening | −107 | 7050000 | 6.85 | |
| 506-0035 | 5 | 1.5 QW + RBV | Day 1 | 1 | 10700000 | 7.03 | |
| 506-0035 | 5 | 1.5 QW + RBV | Day 2 | 2 | 2900000 | 6.46 | 0.57 |
| 506-0035 | 5 | 1.5 QW + RBV | Day 4 | 5 | 919000 | 5.96 | 1.07 |
| 506-0035 | 5 | 1.5 QW + RBV | Day 8 | 8 | 1990000 | 6.30 | 0.73 |
| 506-0035 | 5 | 1.5 QW + RBV | Day 15 | 15 | 227000 | 5.36 | 1.67 |
| 506-0035 | 5 | 1.5 QW + RBV | Day 22 | 22 | 4600 | 3.66 | 3.37 |
| 506-0035 | 5 | 1.5 QW + RBV | Day 29 | 29 | 3080 | 3.49 | 3.54 |
| 506-0035 | 5 | 1.5 QW + RBV | Day 59 | 68 | 2890000 | 6.46 | 0.57 |
| 507-0042 | 5 | 1.5 QW + RBV | Screening | −54 | 42700000 | 7.63 | |
| 507-0042 | 5 | 1.5 QW + RBV | Day 1 | 1 | 32900000 | 7.52 | |
| 507-0042 | 5 | 1.5 QW + RBV | Day 2 | 2 | 2050000 | 6.31 | 1.21 |
| 507-0042 | 5 | 1.5 QW + RBV | Day 4 | 3 | 14500 | 4.16 | 3.36 |
| 507-0042 | 5 | 1.5 QW + RBV | Day 8 | 9 | 234000 | 5.37 | 2.15 |
| 507-0042 | 5 | 1.5 QW + RBV | Day 15 | 17 | 2570 | 3.41 | 4.11 |
| 507-0042 | 5 | 1.5 QW + RBV | Day 22 | 24 | 481 | 2.68 | 4.84 |
| 507-0042 | 5 | 1.5 QW + RBV | Day 29 | 31 | 91 | 1.96 | 5.56 |
| 507-0042 | 5 | 1.5 QW + RBV | Day 59 | 134 | 16800000 | 7.23 | 0.29 |
| 507-0043 | 5 | 1.5 QW + RBV | Screening | −42 | 6000000 | 6.78 | |
| 507-0043 | 5 | 1.5 QW + RBV | Day 1 | 1 | 3480000 | 6.54 | |
| 507-0043 | 5 | 1.5 QW + RBV | Day 2 | 2 | 2060000 | 6.31 | 0.23 |
| 507-0043 | 5 | 1.5 QW + RBV | Day 8 | 9 | 134000 | 5.13 | 1.41 |
| 507-0043 | 5 | 1.5 QW + RBV | Day 29 | 29 | 2000000 | 6.30 | 0.24 |
| 507-0043 | 5 | 1.5 QW + RBV | Day 59 | 56 | 1870000 | 6.27 | 0.27 |
| 502-0054 | 6 | 0.75 QW + RBV | Screening | −42 | 2520000 | 6.4 | |
| 502-0054 | 6 | 0.75 QW + RBV | Day 1 | 1 | 1460000 | 6.16 | |
| 502-0054 | 6 | 0.75 QW + RBV | Day 2 | 2 | 1870000 | 6.27 | −0.11 |
| 502-0054 | 6 | 0.75 QW + RBV | Day 4 | 3 | 2730000 | 6.44 | −0.28 |
| 502-0054 | 6 | 0.75 QW + RBV | Day 8 | 8 | 844000 | 5.93 | 0.23 |
| 502-0054 | 6 | 0.75 QW + RBV | Day 15 | 15 | 247000 | 5.39 | 0.77 |
| 502-0054 | 6 | 0.75 QW + RBV | Day 22 | 22 | 23000 | 4.36 | 1.80 |
| 502-0054 | 6 | 0.75 QW + RBV | Day 29 | 29 | 2640 | 3.42 | 2.74 |
| 502-0054 | 6 | 0.75 QW + RBV | Day 59 | 57 | 2260000 | 6.35 | −0.19 |
| 502-0058 | 6 | 0.75 QW + RBV | Screening | −25 | 20800000 | 7.32 | |
| 502-0058 | 6 | 0.75 QW + RBV | Day 1 | 1 | 16000000 | 7.20 | |
| 502-0058 | 6 | 0.75 QW + RBV | Day 2 | 2 | 207000 | 5.32 | 1.88 |
| 502-0058 | 6 | 0.75 QW + RBV | Day 4 | 4 | 79200 | 4.90 | 2.30 |
| 502-0058 | 6 | 0.75 QW + RBV | Day 8 | 8 | 395000 | 5.60 | 1.60 |
| 502-0058 | 6 | 0.75 QW + RBV | Day 15 | 15 | 130000 | 5.11 | 2.09 |
| 502-0058 | 6 | 0.75 QW + RBV | Day 22 | 22 | 4670 | 3.67 | 3.53 |
| 502-0058 | 6 | 0.75 QW + RBV | Day 29 | 29 | 343 | 2.54 | 4.66 |
| 502-0058 | 6 | 0.75 QW + RBV | Day 59 | 59 | 15000000 | 7.18 | 0.02 |
| 502-0059 | 6 | 0.75 QW + RBV | Screening | −34 | 5270000 | 6.72 | |
| 502-0059 | 6 | 0.75 QW + RBV | Day 1 | 1 | 5630000 | 6.75 | |
| 502-0059 | 6 | 0.75 QW + RBV | Day 2 | 2 | 1960000 | 6.29 | 0.46 |
| 502-0059 | 6 | 0.75 QW + RBV | Day 4 | 4 | 504000 | 5.70 | 1.05 |
| 502-0059 | 6 | 0.75 QW + RBV | Day 8 | 8 | 977000 | 5.99 | 0.76 |
| 502-0059 | 6 | 0.75 QW + RBV | Day 15 | 15 | 421000 | 5.62 | 1.13 |
| 502-0059 | 6 | 0.75 QW + RBV | Day 22 | 22 | 270000 | 5.43 | 1.32 |
| 502-0059 | 6 | 0.75 QW + RBV | Day 29 | 29 | 110000 | 5.04 | 1.71 |
| 502-0059 | 6 | 0.75 QW + RBV | Day 59 | 59 | 1910000 | 6.28 | 0.47 |
| 501-0051 | 7 | 0.5 QW + RBV | Screening | −139 | 1860000 | 6.27 | |
| 501-0051 | 7 | 0.5 QW + RBV | Day 1 | 1 | 3610000 | 6.56 | |
| 501-0051 | 7 | 0.5 QW + RBV | Day 2 | 2 | 375000 | 5.57 | 0.99 |
| 501-0051 | 7 | 0.5 QW + RBV | Day 4 | 3 | 1580 | 3.20 | 3.36 |
| 501-0051 | 7 | 0.5 QW + RBV | Day 8 | 8 | 16100 | 4.21 | 2.35 |
| 501-0051 | 7 | 0.5 QW + RBV | Day 15 | 15 | 11400 | 4.06 | 2.50 |
| 501-0051 | 7 | 0.5 QW + RBV | Day 22 | 23 | 5610 | 3.75 | 2.81 |
| 501-0051 | 7 | 0.5 QW + RBV | Day 29 | 29 | 1770 | 3.25 | 3.31 |
| 501-0051 | 7 | 0.5 QW + RBV | Day 59 | 58 | 1640000 | 6.21 | 0.35 |
| 503-0068 | 7 | 0.5 QW + RBV | Screening | −37 | 7270000 | 6.86 | |
| 503-0068 | 7 | 0.5 QW + RBV | Day 1 | 1 | 3950000 | 6.60 | |
| 503-0068 | 7 | 0.5 QW + RBV | Day 2 | 2 | 1140000 | 6.06 | 0.54 |
| 503-0068 | 7 | 0.5 QW + RBV | Day 4 | 3 | 1000000 | 6.00 | 0.60 |
| 503-0068 | 7 | 0.5 QW + RBV | Day 8 | 7 | 2030000 | 6.31 | 0.29 |
| 503-0068 | 7 | 0.5 QW + RBV | Day 15 | 16 | 790000 | 5.90 | 0.70 |
| 503-0068 | 7 | 0.5 QW + RBV | Day 22 | 22 | 924000 | 5.97 | 0.63 |
| 503-0068 | 7 | 0.5 QW + RBV | Day 29 | 29 | 1990000 | 6.30 | 0.30 |
| 503-0068 | 7 | 0.5 QW + RBV | Day 59 | 62 | 4150000 | 6.62 | −0.02 |
| 503-0074 | 7 | 0.5 QW + RBV | Screening | −16 | 100000 | 5 | |
| 503-0074 | 7 | 0.5 QW + RBV | Day 1 | 1 | 329000 | 5.52 | |
| 503-0074 | 7 | 0.5 QW + RBV | Day 4 | 3 | 34200 | 4.53 | 0.99 |
| 503-0074 | 7 | 0.5 QW + RBV | Day 8 | 7 | 46300 | 4.67 | 0.85 |
| 503-0074 | 7 | 0.5 QW + RBV | Day 15 | 15 | 4470 | 3.65 | 1.87 |
| 503-0074 | 7 | 0.5 QW + RBV | Day 22 | 22 | 1120 | 3.05 | 2.47 |
| 503-0074 | 7 | 0.5 QW + RBV | Day 29 | 29 | 89 | 1.95 | 3.57 |
| 503-0074 | 7 | 0.5 QW + RBV | Day 59 | 55 | 252000 | 5.40 | 0.12 |
| 507-0075 | 7 | 0.5 QW + RBV | Screening | −24 | 158000 | 5.2 | |
| 507-0075 | 7 | 0.5 QW + RBV | Day 1 | 1 | 590000 | 5.77 | |
| 507-0075 | 7 | 0.5 QW + RBV | Day 2 | 2 | 475000 | 5.68 | 0.09 |
| 507-0075 | 7 | 0.5 QW + RBV | Day 4 | 5 | 154000 | 5.19 | 0.58 |
| 507-0075 | 7 | 0.5 QW + RBV | Day 8 | 8 | 344000 | 5.54 | 0.23 |
| 507-0075 | 7 | 0.5 QW + RBV | Day 15 | 15 | 180000 | 5.26 | 0.51 |
| 507-0075 | 7 | 0.5 QW + RBV | Day 22 | 21 | 56000 | 4.75 | 1.02 |
| 507-0075 | 7 | 0.5 QW + RBV | Day 29 | 28 | 10700 | 4.03 | 1.74 |
| 507-0075 | 7 | 0.5 QW + RBV | Day 59 | 57 | 338000 | 5.53 | 0.24 |
| 501-0094 | 8 | 2.25 QW + RBV | Screening | −28 | 746000 | 5.87 | |
| 505-0085 | 8 | 2.25 QW + RBV | Screening | −19 | 11600000 | 7.06 | |
| 505-0085 | 8 | 2.25 QW + RBV | Day 1 | 1 | 8310000 | 6.92 | |
| 505-0085 | 8 | 2.25 QW + RBV | Day 2 | 2 | 794000 | 5.90 | 1.02 |
| 505-0085 | 8 | 2.25 QW + RBV | Day 4 | 3 | 19600 | 4.29 | 2.63 |
| 505-0085 | 8 | 2.25 QW + RBV | Day 8 | 8 | 25800 | 4.41 | 2.51 |
| 505-0085 | 8 | 2.25 QW + RBV | Day 15 | 15 | 1450 | 3.16 | 3.76 |
| 505-0085 | 8 | 2.25 QW + RBV | Day 22 | 22 | 84 | 1.92 | 5.00 |
| 505-0085 | 8 | 2.25 QW + RBV | Day 23 | 23 | 120 | 2.08 | 4.84 |
| 505-0085 | 8 | 2.25 QW + RBV | Day 24 | 24 | 60 | 1.78 | 5.14 |
| 505-0085 | 8 | 2.25 QW + RBV | Day 29 | 29 | 86 | 1.93 | 4.99 |
| 507-0078 | 8 | 2.25 QW + RBV | Screening | −27 | 1190000 | 6.08 | |
| 507-0078 | 8 | 2.25 QW + RBV | Day 1 | 1 | 1310000 | 6.12 | |
| 507-0078 | 8 | 2.25 QW + RBV | Day 2 | 2 | 450000 | 5.65 | 0.47 |
| 507-0078 | 8 | 2.25 QW + RBV | Day 4 | 3 | 20100 | 4.30 | 1.82 |
| 507-0078 | 8 | 2.25 QW + RBV | Day 8 | 8 | 94300 | 4.97 | 1.15 |
| 507-0078 | 8 | 2.25 QW + RBV | Day 15 | 16 | 11400 | 4.06 | 2.06 |
| 507-0078 | 8 | 2.25 QW + RBV | Day 22 | 23 | 2490 | 3.40 | 2.72 |
| 507-0078 | 8 | 2.25 QW + RBV | Day 29 | 31 | 838 | 2.92 | 3.20 |
| 507-0083 | 8 | 2.25 QW + RBV | Screening | −35 | 2080000 | 6.32 | |
| 507-0083 | 8 | 2.25 QW + RBV | Day 1 | 1 | 2960000 | 6.47 | |
| 507-0083 | 8 | 2.25 QW + RBV | Day 2 | 2 | 1210000 | 6.08 | 0.39 |
| 507-0083 | 8 | 2.25 QW + RBV | Day 4 | 3 | 65800 | 4.82 | 1.65 |
| 507-0083 | 8 | 2.25 QW + RBV | Day 8 | 8 | 173000 | 5.24 | 1.23 |
| 507-0083 | 8 | 2.25 QW + RBV | Day 15 | 16 | 6090 | 3.78 | 2.69 |
| 507-0083 | 8 | 2.25 QW + RBV | Day 22 | 23 | 6290 | 3.80 | 2.67 |
| 507-0083 | 8 | 2.25 QW + RBV | Day 29 | 31 | 455 | 2.66 | 3.81 |
| 502-0091 | 9 | 1.5 QW + RBV | Screening | −28 | 1050000 | 6.02 | |
| 502-0091 | 9 | 1.5 QW + RBV | Day 1 | 1 | 758000 | 5.88 | |
| 502-0091 | 9 | 1.5 QW + RBV | Day 2 | 2 | 128000 | 5.11 | 0.77 |
| 502-0091 | 9 | 1.5 QW + RBV | Day 3 | 3 | 55100 | 4.74 | 1.14 |
| 502-0091 | 9 | 1.5 QW + RBV | Day 4 | 4 | 64400 | 4.81 | 1.07 |
| 503-0089 | 9 | 1.5 QW + RBV | Screening | −34 | 608000 | 5.78 | |
| 503-0089 | 9 | 1.5 QW + RBV | Day 1 | 1 | 2690000 | 6.43 | |
| 503-0089 | 9 | 1.5 QW + RBV | Day 2 | 2 | 655000 | 5.82 | 0.61 |
| 503-0089 | 9 | 1.5 QW + RBV | Day 4 | 4 | 455000 | 5.66 | 0.77 |

TABLE 13
Descriptive Statistics for HCV RNA (log scale)
Subjects with Weekly Combination Therapy (PEG-rIL-29 + Ribavirin)
ITT Set

| | | | 0.5 μg/kg QW + | 0.75 μg/kg QW + | 1.5 μg/kg QW + | 2.25 μg/kg QW + | 1.5 μg/kg QW + |
|---|---|---|---|---|---|---|---|
| | | | RBV | RBV | RBV | RBV | RBV (naive) |
| Visit | Variable | | (N = 4) | (N = 3) | (N = 6) | (N = 4) | (N = 2) |
| Baseline | Result | n | 4 | 3 | 6 | 4 | 2 |
| | | Mean | 6.113 (0.550) | 6.703 (0.522) | 6.695 (0.596) | 6.345 (0.456) | 6.155 (0.389) |
| | | (SD) | | | | | |
| | | Median | 6.165 | 6.750 | 6.750 | 6.295 | 6.155 |
| | | Min, | 5.52, 6.60 | 6.16, 7.20 | 5.89, 7.52 | 5.87, 6.92 | 5.88, 6.43 |
| | | Max | | | | | |
| Day 2 | Result | n | 3 | 3 | 6 | 3 | 2 |
| | | Mean | 5.770 (0.257) | 5.960 (0.554) | 5.887 (0.442) | 5.877 (0.216) | 5.465 (0.502) |
| | | (SD) | | | | | |
| | | Median | 5.680 | 6.270 | 5.870 | 5.900 | 5.465 |
| | | Min, | 5.57, 6.06 | 5.32, 6.29 | 5.39, 6.46 | 5.65, 6.08 | 5.11, 5.82 |
| | | Max | | | | | |
| | Change from | n | 3 | 3 | 6 | 3 | 2 |
| | Baseline | Mean | −0.54 (0.45) | −0.74 (1.02) | −0.81 (0.44) | −0.63 (0.34) | −0.69 (0.11) |
| | | (SD) | | | | | |
| | | Median | −0.54 | −0.46 | −0.95 | −0.47 | −0.69 |
| | | Min, | −1.0, −0.1 | −1.9, 0.1 | −1.2, −0.1 | −1.0, −0.4 | −0.8, −0.6 |
| | | Max | | | | | |
| Day 3 | Result | n | | | | | 1 |
| | | Mean | | | | | 4.740 (—) |
| | | (SD) | | | | | |
| | | Median | | | | | 4.740 |
| | | Min, | | | | | 4.74, 4.74 |
| | | Max | | | | | |
| | Change from | n | | | | | 1 |
| | Baseline | Mean | | | | | −1.14 (—) |
| | | (SD) | | | | | |
| | | Median | | | | | −1.14 |
| | | Min, | | | | | −1.1, −1.1 |
| | | Max | | | | | |
| Day 4 | Result | n | 4 | 3 | 6 | 3 | 2 |
| | | Mean | 4.730 (1.184) | 5.680 (0.770) | 5.073 (0.702) | 4.470 (0.303) | 5.235 (0.601) |
| | | (SD) | | | | | |
| | | Median | 4.860 | 5.700 | 4.895 | 4.300 | 5.235 |
| | | Min, | 3.20, 6.00 | 4.90, 6.44 | 4.16, 5.96 | 4.29, 4.82 | 4.81, 5.66 |
| | | Max | | | | | |
| | Change from | n | 4 | 3 | 6 | 3 | 2 |
| | Baseline | Mean | −1.38 (1.33) | −1.02 (1.29) | −1.62 (1.14) | −2.03 (0.52) | −0.92 (0.21) |
| | | (SD) | | | | | |
| | | Median | −0.80 | −1.05 | −1.48 | −1.82 | −0.92 |
| | | Min, | −3.4, −0.6 | −2.3, 0.3 | −3.4, −0.1 | −2.6, −1.7 | −1.1, −0.8 |
| | | Max | | | | | |
| Day 8 | Result | n | 4 | 3 | 6 | 3 | |
| | | Mean | 5.183 (0.932) | 5.840 (0.210) | 5.648 (0.429) | 4.873 (0.423) | |
| | | (SD) | | | | | |
| | | Median | 5.105 | 5.930 | 5.605 | 4.970 | |
| | | Min, | 4.21, 6.31 | 5.60, 5.99 | 5.12, 6.30 | 4.41, 5.24 | |
| | | Max | | | | | |
| | Change from | n | 4 | 3 | 6 | 3 | |
| | Baseline | Mean | −0.93 (0.99) | −0.86 (0.69) | −1.05 (0.74) | −1.63 (0.76) | |
| | | (SD) | | | | | |
| | | Median | −0.57 | −0.76 | −0.92 | −1.23 | |
| | | Min, | −2.4, −0.2 | −1.6, −0.2 | −2.2, −0.1 | −2.5, −1.2 | |
| | | Max | | | | | |
| Day | Result | n | 4 | 3 | 6 | 3 | |
| 15 | | Mean | 4.718 (1.043) | 5.373 (0.255) | 4.562 (1.058) | 3.667 (0.461) | |
| | | (SD) | | | | | |
| | | Median | 4.660 | 5.390 | 4.710 | 3.780 | |
| | | Min, | 3.65, 5.90 | 5.11, 5.62 | 3.27, 5.91 | 3.16, 4.06 | |
| | | Max | | | | | |
| | Change from | n | 4 | 3 | 6 | 3 | |
| | Baseline | Mean | −1.40 (0.95) | −1.33 (0.68) | −2.13 (1.41) | −2.84 (0.86) | |
| | | (SD) | | | | | |
| | | Median | −1.29 | −1.13 | −2.09 | −2.69 | |
| | | Min, | −2.5, −0.5 | −2.1, −0.8 | −4.1, 0.0 | −3.8, −2.1 | |
| | | Max | | | | | |
| Day | Result | n | 4 | 3 | 6 | 3 | |
| 22 | | Mean | 4.380 (1.269) | 4.487 (0.887) | 3.872 (1.127) | 3.040 (0.990) | |
| | | (SD) | | | | | |
| | | Median | 4.250 | 4.360 | 3.705 | 3.400 | |
| | | Min, | 3.05, 5.97 | 3.67, 5.43 | 2.68, 5.83 | 1.92, 3.80 | |
| | | Max | | | | | |
| | Change from | n | 4 | 3 | 6 | 3 | |
| | Baseline | Mean | −1.73 (1.07) | −2.22 (1.16) | −2.82 (1.60) | −3.46 (1.33) | |
| | | (SD) | | | | | |
| | | Median | −1.75 | −1.80 | −3.25 | −2.72 | |
| | | Min, | −2.8, −0.6 | −3.5, −1.3 | −4.8, −0.1 | −5.0, −2.7 | |
| | | Max | | | | | |
| Day | Result | n | | | | 1 | |
| 23 | | Mean | | | | 2.080 (—) | |
| | | (SD) | | | | | |
| | | Median | | | | 2.080 | |
| | | Min, | | | | 2.08, 2.08 | |
| | | Max | | | | | |
| | Change from | n | | | | 1 | |
| | Baseline | Mean | | | | −4.84 (—) | |
| | | (SD) | | | | | |
| | | Median | | | | −4.84 | |
| | | Min, | | | | −4.8, −4.8 | |
| | | Max | | | | | |
| Day | Result | n | | | | 1 | |
| 24 | | Mean | | | | 1.780 (—) | |
| | | (SD) | | | | | |
| | | Median | | | | 1.780 | |
| | | Min, | | | | 1.78, 1.78 | |
| | | Max | | | | | |
| | Change from | n | | | | 1 | |
| | Baseline | Mean | | | | −5.14 (.) | |
| | | (SD) | | | | | |
| | | Median | | | | −5.14 | |
| | | Min, | | | | −5.1, −5.1 | |
| | | Max | | | | | |
| Day | Result | n | 4 | 3 | 6 | 3 | |
| 29 | | Mean | 3.883 (1.826) | 3.667 (1.268) | 3.468 (1.456) | 2.503 (0.513) | |
| | | (SD) | | | | | |
| | | Median | 3.640 | 3.420 | 3.365 | 2.660 | |
| | | Min, | 1.95, 6.30 | 2.54, 5.04 | 1.96, 5.93 | 1.93, 2.92 | |
| | | Max | | | | | |
| | Change from | n | 4 | 3 | 6 | 3 | |
| | Baseline | Mean | −2.23 (1.52) | −3.04 (1.50) | −3.23 (1.89) | −4.00 (0.91) | |
| | | (SD) | | | | | |
| | | Median | −2.53 | −2.74 | −3.65 | −3.81 | |
| | | Min, | −3.6, −0.3 | −4.7, −1.7 | −5.6, 0.0 | −5.0, −3.2 | |
| | | Max | | | | | |
| Day | Result | n | 4 | 3 | 6 | | |
| 59 | | Mean | 5.940 (0.576) | 6.603 (0.501) | 6.405 (0.495) | | |
| | | (SD) | | | | | |
| | | Median | 5.870 | 6.350 | 6.395 | | |
| | | Min, | 5.40, 6.62 | 6.28, 7.18 | 5.90, 7.23 | | |
| | | Max | | | | | |
| | Change from | n | 4 | 3 | 6 | | |
| | Baseline | Mean | −0.17 (0.16) | −0.10 (0.34) | −0.29 (0.20) | | |
| | | (SD) | | | | | |
| | | Median | −0.18 | −0.02 | −0.31 | | |
| | | Min, | −0.4, 0.0 | −0.5, 0.2 | −0.6, 0.0 | | |
| | | Max | | | | | |
| Max Decr from | | n | 4 | 3 | 6 | 3 | 2 |
| Baseline | | Mean | −2.343 (1.367) | −3.037 (1.497) | −3.248 (1.849) | −4.050 (0.992) | −0.955 (0.262) |
| | | (SD) | | | | | |
| | | 95% CI | −4.52, −0.17 | −6.76, 0.68 | −5.19, −1.31 | −6.51, −1.59 | −3.31, 1.40 |
| | | Median | −2.550 | −2.740 | −3.645 | −3.810 | −0.955 |
| | | Min, | −3.57, −0.70 | −4.66, −1.71 | −5.56, −0.09 | −5.14, −3.20 | −1.14, −0.77 |
| | | Max | | | | | |

a Rbv = Ribavirin
Note:
Lower limit of detection for assay is 25 IU/ml (log scale = 1.4)

TABLE 14
Incidence of Adverse Events by Preferred Term, Sorted
by Decreasing Frequency Safety Analysis Set

| | 1.5 μg/ | 3.0 μg/ | 1.5 μg/ | |
|---|---|---|---|---|
| | kg Q2W | kg Q2W | kg QW | Total |
| | (N = 6) | (N = 6) | (N = 6) | (N = 18) |
| Preferred Term^{a} | n (%) | n (%) | n (%) | n (%) |
| Any AE | 2 (33) | 3 (50) | 2 (33) | 7 (39) |
| Fatigue | 1 (17) | 1 (17) | 1 (17) | 3 (17) |
| Myalgia | 1 (17) | 0 (0) | 1 (17) | 2 (11) |
| Abdominal discomfort | 1 (17) | 0 (0) | 0 (0) | 1 (6) |
| Abdominal pain upper | 1 (17) | 0 (0) | 0 (0) | 1 (6) |
| Anorexia | 0 (0) | 1 (17) | 0 (0) | 1 (6) |
| Arthritis | 1 (17) | 0 (0) | 0 (0) | 1 (6) |
| Cough | 0 (0) | 0 (0) | 1 (17) | 1 (6) |
| Diarrhoea | 1 (17) | 0 (0) | 0 (0) | 1 (6) |
| Dysgeusia | 0 (0) | 1 (17) | 0 (0) | 1 (6) |
| Influenza like illness | 0 (0) | 1 (17) | 0 (0) | 1 (6) |
| Injection site erythema | 0 (0) | 0 (0) | 1 (17) | 1 (6) |
| Irritability | 1 (17) | 0 (0) | 0 (0) | 1 (6) |
| Nausea | 0 (0) | 1 (17) | 0 (0) | 1 (6) |
| Pharyngolaryngeal pain | 1 (17) | 0 (0) | 0 (0) | 1 (6) |
| Pneumonia | 1 (17) | 0 (0) | 0 (0) | 1 (6) |
| Pyrexia | 1 (17) | 0 (0) | 0 (0) | 1 (6) |
| Sunburn | 0 (0) | 1 (17) | 0 (0) | 1 (6) |
| Upper respiratory tract | 1 (17) | 0 (0) | 0 (0) | 1 (6) |
| infection | | | | |

^{a}MedDRA version 11.0 or higher
Sort order based on Total column

TABLE 15
Incidence of Adverse Events by Preferred Term, Sorted by Decreasing Frequency
Subjects with Weekly Combination Therapy (PEG-rIL-29 + Ribavirin)

| | 0.5 μg/kg | 0.75 μg/kg | 1.5 μg/kg | 2.25 μg/kg | 1.5 μg/kg (Naive) | Total^{b} |
|---|---|---|---|---|---|---|
| | (N = 4) | (N = 3) | (N = 7) | (N = 4) | (N = 2) | (N = 20) |
| Preferred Term^{a} | n (%) | n (%) | n (%) | n (%) | n (%) | n (%) |
| Any AE | 2 (50) | 1 (33) | 6 (86) | 0 | 0 | 9 (45) |
| Fatigue | 1 (25) | 0 | 3 (43) | 0 | 0 | 4 (20) |
| Nausea | 1 (25) | 0 | 3 (43) | 0 | 0 | 4 (20) |
| Insomnia | 0 | 0 | 3 (43) | 0 | 0 | 3 (15) |
| Chills | 0 | 0 | 2 (29) | 0 | 0 | 2 (10) |
| Decreased appetite | 1 (25) | 0 | 1 (14) | 0 | 0 | 2 (10) |
| Influenza like illness | 0 | 0 | 2 (29) | 0 | 0 | 2 (10) |
| Pruritus | 1 (25) | 0 | 1 (14) | 0 | 0 | 2 (10) |
| Acute respiratory distress syndrome | 0 | 0 | 1 (14) | 0 | 0 | 1 (5) |
| Adverse drug reaction | 0 | 0 | 1 (14) | 0 | 0 | 1 (5) |
| Blood amylase increased | 0 | 0 | 1 (14) | 0 | 0 | 1 (5) |
| Chronic obstructive pulmonary disease | 0 | 0 | 1 (14) | 0 | 0 | 1 (5) |
| Cough | 0 | 0 | 1 (14) | 0 | 0 | 1 (5) |
| Diarrhoea | 0 | 0 | 1 (14) | 0 | 0 | 1 (5) |
| Dry mouth | 1 (25) | 0 | 0 | 0 | 0 | 1 (5) |
| Dysgeusia | 0 | 0 | 1 (14) | 0 | 0 | 1 (5) |
| Headache | 0 | 0 | 1 (14) | 0 | 0 | 1 (5) |
| Hepatotoxicity | 0 | 0 | 1 (14) | 0 | 0 | 1 (5) |
| Hyperglycaemia | 0 | 0 | 1 (14) | 0 | 0 | 1 (5) |
| Injection site haematoma | 0 | 0 | 1 (14) | 0 | 0 | 1 (5) |
| Irritability | 0 | 1 (33) | 0 | 0 | 0 | 1 (5) |
| Lipase increased | 0 | 0 | 1 (14) | 0 | 0 | 1 (5) |
| Migraine | 0 | 0 | 1 (14) | 0 | 0 | 1 (5) |
| Myalgia | 0 | 0 | 1 (14) | 0 | 0 | 1 (5) |
| Nasal congestion | 0 | 0 | 1 (14) | 0 | 0 | 1 (5) |
| Oropharyngeal pain | 1 (25) | 0 | 0 | 0 | 0 | 1 (5) |
| Pneumonia | 0 | 0 | 1 (14) | 0 | 0 | 1 (5) |
| Sinusitis | 0 | 0 | 1 (14) | 0 | 0 | 1 (5) |

^{a}MedDRA version 11.0 or higher
^{b}Sort order based on Total column

TABLE 16
Laboratory - Hematology: Red Blood Cell Indices/Platelets Standard Units

| | | | Study | Hematocrit | Hemoglobin | RBC | RDW | MCV | Platelets |
|---|---|---|---|---|---|---|---|---|---|
| Subject | Treatment | Visit | Day | (fract of 1) | (g/L) | (10E12/L) | (fract of 1) | (fL) | (10E9/L) |
| 501-0015 | 3.0 μg/kg Q2W | Screening | −61 | 0.390 | 125 | 4.52 | 0.146 (H) | 86.0 | 320 |
| 501-0015 | 3.0 μg/kg Q2W | Day 1 | 1 | 0.420 | 135 | 4.86 | 0.147 (H) | 86.0 | 316 |
| 501-0015 | 3.0 μg/kg Q2W | Day 8 | 8 | 0.390 | 125 | 4.53 | 0.148 (H) | 86.0 | 316 |
| 501-0015 | 3.0 μg/kg Q2W | Day 15 | 15 | 0.400 | 125 | 4.57 | 0.147 (H) | 87.0 | 324 |
| 501-0015 | 3.0 μg/kg Q2W | Day 22 | 22 | 0.380 | 124 | 4.44 | 0.149 (H) | 85.0 | 304 |
| 501-0015 | 3.0 μg/kg Q2W | Day 29 | 31 | 0.410 | 130 | 4.75 | 0.147 (H) | 85.0 | 300 |
| 501-0015 | 3.0 μg/kg Q2W | Day 59 | 66 | 0.400 | 129 | 4.66 | 0.148 (H) | 86.0 | 329 |
| 501-0017 | 3.0 μg/kg Q2W | Screening | −33 | 0.470 (H) | 163 (H) | 5.10 | 0.129 | 92.0 | 305 |
| 501-0017 | 3.0 μg/kg Q2W | Day 1 | 1 | 0.460 (H) | 157 (H) | 4.97 | 0.130 | 93.0 | 270 |
| 501-0017 | 3.0 μg/kg Q2W | Day 8 | 8 | 0.440 | 154 | 4.81 | 0.128 | 92.0 | 285 |
| 501-0017 | 3.0 μg/kg Q2W | Day 15 | 15 | 0.440 | 148 | 4.69 | 0.128 | 93.0 | 258 |
| 501-0017 | 3.0 μg/kg Q2W | Day 22 | 22 | 0.450 | 155 | 4.82 | 0.134 | 93.0 | 250 |
| 501-0017 | 3.0 μg/kg Q2W | Day 29 | 29 | 0.460 (H) | 155 | 4.89 | 0.135 | 94.0 | 256 |
| 501-0017 | 3.0 μg/kg Q2W | Day 59 | 57 | 0.470 (H) | 160 (H) | 4.97 | 0.129 | 94.0 | 262 |
| 501-0021 | 3.0 μg/kg Q2W | Screening | −26 | 0.450 | 157 (H) | 4.88 | 0.138 | 92.0 | 190 |
| 501-0021 | 3.0 μg/kg Q2W | Day 1 | 1 | 0.460 (H) | 152 | 4.84 | 0.137 | 94.0 | 188 |
| 501-0021 | 3.0 μg/kg Q2W | Day 8 | 8 | 0.450 | 152 | 4.84 | 0.142 | 93.0 | 200 |
| 501-0021 | 3.0 μg/kg Q2W | Day 15 | 15 | 0.470 (H) | 159 (H) | 4.98 | 0.140 | 95.0 | 194 |
| 501-0021 | 3.0 μg/kg Q2W | Day 22 | 22 | 0.440 | 153 | 4.83 | 0.140 | 92.0 | 176 |
| 501-0021 | 3.0 μg/kg Q2W | Day 29 | 31 | 0.450 | 154 | 4.88 | 0.139 | 93.0 | 182 |
| 501-0021 | 3.0 μg/kg Q2W | Day 59 | 59 | 0.450 | 155 | 4.79 | 0.136 | 93.0 | 184 |
| 502-0001 | 1.5 μg/kg Q2W | Screening | −26 | 0.417 | 142 | 4.47 | 0.119 | 93.0 | 297 |
| 502-0001 | 1.5 μg/kg Q2W | Day 1 | 1 | 0.388 | 138 | 4.18 | 0.127 | 93.0 | 270 |
| 502-0001 | 1.5 μg/kg Q2W | Day 8 | 8 | 0.383 | 133 | 4.10 | 0.126 | 93.0 | 297 |
| 502-0001 | 1.5 μg/kg Q2W | Day 15 | 15 | 0.399 | 137 | 4.26 | 0.123 | 94.0 | 302 |
| 502-0001 | 1.5 μg/kg Q2W | Day 22 | 22 | 0.383 | 135 | 4.12 | 0.123 | 93.0 | 290 |
| 502-0001 | 1.5 μg/kg Q2W | Day 29 | 29 | 0.372 | 129 | 3.97 | 0.121 | 94.0 | 281 |
| 502-0001 | 1.5 μg/kg Q2W | Day 59 | 59 | 0.428 | 144 | 4.50 | 0.123 | 95.0 | 291 |
| 502-0003 | 1.5 μg/kg Q2W | Screening | −25 | 0.468 | 166 | 5.06 | 0.132 | 93.0 | 193 |
| 502-0003 | 1.5 μg/kg Q2W | Day 1 | 1 | 0.493 | 171 (H) | 5.20 | 0.140 | 95.0 | 173 |
| 502-0003 | 1.5 μg/kg Q2W | Day 8 | 8 | 0.459 | 162 | 4.88 | 0.139 | 94.0 | 158 |
| 502-0003 | 1.5 μg/kg Q2W | Day 15 | 15 | 0.492 | 171 (H) | 5.16 | 0.137 | 95.0 | 212 |
| 502-0003 | 1.5 μg/kg Q2W | Day 22 | 22 | 0.462 | 161 | 4.89 | 0.136 | 95.0 | 214 |
| 502-0003 | 1.5 μg/kg Q2W | Day 29 | 29 | 0.468 | 166 | 4.98 | 0.138 | 94.0 | 174 |
| 502-0003 | 1.5 μg/kg Q2W | Day 59 | 59 | 0.452 | 160 | 4.89 | 0.134 | 92.0 | 205 |
| 502-0008 | 1.5 μg/kg Q2W | Screening | −22 | 0.446 | 153 | 5.06 | 0.123 | 88.0 | 293 |
| 502-0008 | 1.5 μg/kg Q2W | Day 1 | 1 | 0.477 | 160 | 5.40 | 0.126 | 88.0 | 253 |
| 502-0008 | 1.5 μg/kg Q2W | Day 8 | 8 | 0.450 | 151 | 5.09 | 0.129 | 88.0 | 296 |
| 502-0008 | 1.5 μg/kg Q2W | Day 15 | 15 | 0.449 | 149 | 4.91 | 0.132 | 92.0 | 298 |
| 502-0008 | 1.5 μg/kg Q2W | Day 22 | 22 | 0.434 | 148 | 5.03 | 0.129 | 86.0 | 258 |
| 502-0008 | 1.5 μg/kg Q2W | Day 29 | 29 | 0.454 | 149 | 5.07 | 0.128 | 90.0 | 289 |
| 502-0008 | 1.5 μg/kg Q2W | Day 59 | 59 | 0.441 | 149 | 5.04 | 0.127 | 88.0 | 290 |
| 502-0009 | 1.5 μg/kg Q2W | Screening | −18 | 0.440 | 158 (H) | 5.02 | 0.126 | 88.0 | 224 |
| 502-0009 | 1.5 μg/kg Q2W | Day 1 | 1 | 0.422 | 147 | 4.72 | 0.134 | 89.0 | 212 |
| 502-0009 | 1.5 μg/kg Q2W | Day 8 | 8 | 0.435 | 147 | 4.69 | 0.133 | 93.0 | 189 |
| 502-0009 | 1.5 μg/kg Q2W | Day 15 | 16 | 0.434 | 147 | 4.70 | 0.132 | 92.0 | 232 |
| 502-0009 | 1.5 μg/kg Q2W | Day 22 | 22 | 0.423 | 146 | 4.77 | 0.137 | 89.0 | 202 |
| 502-0009 | 1.5 μg/kg Q2W | Day 29 | 29 | 0.434 | 144 | 4.70 | 0.139 | 92.0 | 219 |
| 502-0009 | 1.5 μg/kg Q2W | Day 59 | 59 | 0.434 | 148 | 4.83 | 0.130 | 90.0 | 216 |
| 502-0012 | 1.5 μg/kg Q2W | Screening | −20 | 0.443 | 154 | 4.77 | 0.141 | 93.0 | 290 |
| 502-0012 | 1.5 μg/kg Q2W | Day 1 | 1 | 0.428 | 147 | 4.40 | 0.150 | 97.0 | 196 |
| 502-0012 | 1.5 μg/kg Q2W | Day 8 | 8 | 0.442 | 152 | 4.61 | 0.148 | 96.0 | 240 |
| 502-0012 | 1.5 μg/kg Q2W | Day 15 | 15 | 0.419 | 146 | 4.32 | 0.157 (H) | 97.0 | 251 |
| 502-0012 | 1.5 μg/kg Q2W | Day 22 | 22 | 0.429 | 148 | 4.42 | 0.157 (H) | 97.0 | 262 |
| 502-0012 | 1.5 μg/kg Q2W | Day 29 | 29 | 0.439 | 150 | 4.43 | 0.151 (H) | 99.0 (H) | 239 |
| 502-0012 | 1.5 μg/kg Q2W | Day 59 | 59 | 0.454 | 153 | 4.44 | 0.136 | 102.0 (H) | 215 |
| 502-0013 | 3.0 μg/kg Q2W | Screening | −61 | 0.451 | 155 | 4.74 | 0.128 | 95.0 | 175 |
| 502-0013 | 3.0 μg/kg Q2W | Day 1 | 1 | 0.464 | 152 | 4.75 | 0.131 | 98.0 | 165 |
| 502-0013 | 3.0 μg/kg Q2W | Day 8 | 8 | 0.462 | 154 | 4.83 | 0.132 | 96.0 | 178 |
| 502-0013 | 3.0 μg/kg Q2W | Day 15 | 14 | 0.450 | 153 | 4.65 | 0.125 | 97.0 | 163 |
| 502-0013 | 3.0 μg/kg Q2W | Day 22 | 22 | 0.462 | 156 | 4.85 | 0.135 | 95.0 | 171 |
| 502-0013 | 3.0 μg/kg Q2W | Day 29 | 29 | 0.449 | 149 | 4.59 | 0.134 | 98.0 | 183 |
| 502-0013 | 3.0 μg/kg Q2W | Day 59 | 59 | 0.467 | 159 | 4.89 | 0.131 | 96.0 | 179 |
| 502-0019 | 3.0 μg/kg Q2W | Screening | −28 | 0.425 | 145 | 4.95 | 0.145 | 86.0 | 223 |
| 502-0019 | 3.0 μg/kg Q2W | Day 1 | 1 | 0.464 (H) | 149 | 5.16 (H) | 0.144 | 90.0 | 206 |
| 502-0019 | 3.0 μg/kg Q2W | Day 8 | 8 | 0.392 | 134 | 4.60 | 0.144 | 85.0 | 203 |
| 502-0019 | 3.0 μg/kg Q2W | Day 15 | 14 | 0.422 | 147 | 4.98 | 0.129 | 85.0 | 226 |
| 502-0019 | 3.0 μg/kg Q2W | Day 22 | 22 | 0.432 | 144 | 4.97 | 0.146 | 87.0 | 200 |
| 502-0019 | 3.0 μg/kg Q2W | Day 29 | 29 | 0.411 | 135 | 4.61 | 0.147 | 89.0 | 206 |
| 502-0019 | 3.0 μg/kg Q2W | Day 59 | 59 | 0.416 | 139 | 4.76 | 0.141 | 88.0 | 223 |
| 502-0020 | 3.0 μg/kg Q2W | Screening | −27 | 0.400 | 136 | 4.14 | 0.129 | 97.0 | 297 |
| 502-0020 | 3.0 μg/kg Q2W | Day 1 | 1 | 0.405 | 136 | 4.09 | 0.128 | 99.0 (H) | 265 |
| 502-0020 | 3.0 μg/kg Q2W | Day 8 | 8 | 0.387 | 133 | 4.02 | 0.128 | 96.0 | 250 |
| 502-0020 | 3.0 μg/kg Q2W | Day 15 | 14 | 0.376 | 133 | 3.96 | 0.117 | 95.0 | 291 |
| 502-0020 | 3.0 μg/kg Q2W | Day 22 | 22 | 0.376 | 128 | 3.92 | 0.129 | 96.0 | 257 |
| 502-0020 | 3.0 μg/kg Q2W | Day 29 | 29 | 0.384 | 129 | 3.87 | 0.130 | 99.0 (H) | 265 |
| 502-0020 | 3.0 μg/kg Q2W | Day 59 | 59 | 0.403 | 132 | 4.07 | 0.131 | 99.0 (H) | 272 |
| 502-0023 | 1.5 μg/kg QW | Screening | −34 | 0.365 | 125 | 3.87 | 0.128 | 95.0 | 171 |
| 502-0023 | 1.5 μg/kg QW | Day 1 | 1 | 0.403 | 135 | 4.18 | 0.128 | 96.0 | 162 |
| 502-0023 | 1.5 μg/kg QW | Day 8 | 7 | 0.387 | 137 | 4.06 | 0.114 (L) | 95.0 | 178 |
| 502-0023 | 1.5 μg/kg QW | Day 15 | 14 | 0.390 | 135 | 4.09 | 0.117 | 95.0 | 190 |
| 502-0023 | 1.5 μg/kg QW | Day 22 | 21 | 0.393 | 138 | 4.14 | 0.114 (L) | 95.0 | 205 |
| 502-0023 | 1.5 μg/kg QW | Day 29 | 29 | 0.374 | 129 | 3.96 | 0.124 | 94.0 | 181 |
| 502-0023 | 1.5 μg/kg QW | Day 36 | 36 | 0.408 | 142 | 4.25 | 0.129 | 96.0 | 182 |
| 502-0023 | 1.5 μg/kg QW | Day 59 | 59 | 0.387 | 130 | 4.08 | 0.126 | 95.0 | 185 |
| 502-0024 | 1.5 μg/kg QW | Screening | −26 | 0.435 | 142 | 4.89 | 0.154 (H) | 89.0 | 197 |
| 502-0024 | 1.5 μg/kg QW | Day 1 | 1 | 0.402 | 133 | 4.56 | 0.152 (H) | 88.0 | 259 |
| 502-0024 | 1.5 μg/kg QW | Day 8 | 7 | 0.394 | 135 | 4.50 | 0.135 | 88.0 | 233 |
| 502-0024 | 1.5 μg/kg QW | Day 15 | 14 | 0.444 | 148 | 5.09 | 0.139 | 87.0 | 228 |
| 502-0024 | 1.5 μg/kg QW | Day 22 | 21 | 0.403 | 139 | 4.67 | 0.138 | 86.0 | 230 |
| 502-0024 | 1.5 μg/kg QW | Day 29 | 29 | 0.398 | 136 | 4.49 | 0.155 (H) | 89.0 | 220 |
| 502-0024 | 1.5 μg/kg QW | Day 36 | 36 | 0.410 | 138 | 4.67 | 0.156 (H) | 88.0 | 291 |
| 502-0024 | 1.5 μg/kg QW | Day 59 | 59 | 0.437 | 143 | 4.91 | 0.154 (H) | 89.0 | 277 |
| 503-0022 | 1.5 μg/kg QW | Screening | −47 | 0.420 | 148 | 4.80 | 0.143 | 87.0 | 195 |
| 503-0022 | 1.5 μg/kg QW | Day 1 | 1 | 0.423 | 145 | 4.80 | 0.140 | 87.4 | 187 |
| 503-0022 | 1.5 μg/kg QW | Day 15 | 15 | 0.428 | 147 | 4.90 | 0.141 | 87.7 | 182 |
| 503-0022 | 1.5 μg/kg QW | Day 22 | 22 | 0.418 | 144 | 4.80 | 0.139 | 87.3 | 181 |
| 503-0022 | 1.5 μg/kg QW | Day 29 | 29 | 0.421 | 147 | 4.90 | 0.141 | 86.3 | 181 |
| 503-0022 | 1.5 μg/kg QW | Day 36 | 38 | 0.433 | 149 | 5.00 | 0.136 | 87.0 | 200 |
| 503-0022 | 1.5 μg/kg QW | Day 59 | 57 | 0.415 | 145 | 4.80 | 0.140 | 87.0 | 196 |
| 505-0006 | 1.5 μg/kg Q2W | Screening | −33 | 0.449 | 159 | 5.00 | 0.126 | 89.8 | 211 |
| 505-0006 | 1.5 μg/kg Q2W | Day 1 | 1 | 0.431 | 152 | 4.91 | 0.126 | 87.7 | 203 |
| 505-0006 | 1.5 μg/kg Q2W | Day 8 | 8 | 0.443 | 158 | 4.97 | 0.127 | 89.1 | 191 |
| 505-0006 | 1.5 μg/kg Q2W | Day 15 | 16 | 0.433 | 152 | 4.86 | 0.127 | 89.1 | 175 |
| 505-0006 | 1.5 μg/kg Q2W | Day 22 | 23 | 0.418 | 149 | 4.74 | 0.128 | 88.2 | 322 |
| 505-0006 | 1.5 μg/kg Q2W | Day 29 | 29 | 0.418 | 147 | 4.64 | 0.129 | 89.9 | 323 |
| 505-0006 | 1.5 μg/kg Q2W | Day 59 | 59 | 0.432 | 151 | 4.83 | 0.136 | 89.4 | 205 |
| 505-0027 | 1.5 μg/kg QW | Screening | −19 | 0.380 | 131 | 4.62 | 0.120 | 82.3 | 237 |
| 505-0027 | 1.5 μg/kg QW | Day 1 | 1 | 0.379 | 129 | 4.57 | 0.126 | 83.0 | 213 |
| 505-0027 | 1.5 μg/kg QW | Day 8 | 8 | 0.387 | 131 | 4.68 | 0.123 | 82.8 | 260 |
| 505-0027 | 1.5 μg/kg QW | Day 15 | 15 | 0.380 | 128 | 4.58 | 0.130 | 82.9 | 254 |
| 505-0027 | 1.5 μg/kg QW | Day 22 | 22 | 0.365 | 124 | 4.43 | 0.122 | 82.4 | 258 |
| 505-0027 | 1.5 μg/kg QW | Day 29 | 29 | 0.380 | 129 | 4.62 | 0.126 | 82.3 | 250 |
| 505-0027 | 1.5 μg/kg QW | Day 36 | 38 | 0.383 | 127 | 4.60 | 0.123 | 83.3 | 242 |
| 505-0027 | 1.5 μg/kg QW | Day 59 | 59 | 0.373 | 128 | 4.50 | 0.122 | 82.9 | 226 |
| 506-0032 | 1.5 μg/kg QW | Screening | −51 | 0.468 | 159 | 5.00 | 13.1 (U) | 93.0 | 177 |
| 506-0032 | 1.5 μg/kg QW | Day 1 | 1 | 0.454 | 160 | 4.89 | 13.3 (U) | 93.0 | 211 |
| 506-0032 | 1.5 μg/kg QW | Day 8 | 8 | 0.447 | 157 | 4.87 | 13.4 (U) | 92.0 | 193 |
| 506-0032 | 1.5 μg/kg QW | Day 15 | 15 | 0.467 | 164 | 5.05 | 13.1 (U) | 92.0 | 171 |
| 506-0032 | 1.5 μg/kg QW | Day 22 | 23 | 0.464 | 161 | 5.00 | 13.9 (U) | 93.0 | 141 |
| 506-0032 | 1.5 μg/kg QW | Day 29 | 29 | 0.476 | 168 | 5.16 | 14.0 (U) | 92.0 | 171 |
| 506-0032 | 1.5 μg/kg QW | Day 36 | 36 | 0.464 | 164 | 4.98 | 13.2 (U) | 93.0 | 172 |
| 507-0028 | 1.5 μg/kg QW | Screening | −40 | 0.443 | 152 | 4.88 | 0.137 | 90.8 | 144 |
| 507-0028 | 1.5 μg/kg QW | Unscheduled | −22 | 0.411 (L) | 144 | 4.53 (L) | 0.129 | 90.7 | 264 |
| 507-0028 | 1.5 μg/kg QW | Day 1 | 1 | 0.424 | 144 | 4.64 (L) | 0.138 | 91.4 | 140 |
| 507-0028 | 1.5 μg/kg QW | Day 8 | 8 | 0.416 (L) | 142 | 4.50 (L) | 0.140 | 92.4 | 149 |
| 507-0028 | 1.5 μg/kg QW | Day 15 | 15 | 0.430 | 147 | 4.66 (L) | 0.138 | 92.3 | 148 |
| 507-0028 | 1.5 μg/kg QW | Day 22 | 22 | 0.424 | 145 | 4.62 (L) | 0.138 | 91.8 | 140 |
| 507-0028 | 1.5 μg/kg QW | Day 29 | 30 | 0.426 | 144 | 4.59 (L) | 0.141 | 92.8 | 149 |
| 507-0028 | 1.5 μg/kg QW | Day 36 | 37 | 0.416 (L) | 142 | 4.47 (L) | 0.138 | 93.1 | 166 |

Note:
L = below lower limit of reference range,
H = above upper limit of reference range,
M = missing reference range,
U = Collected units are unknown
If Flag = M or U then the collected result was not converted to standard result. The collected result is displayed.
Numbers following the L/H flags indicate grades based on CTCAE grading criteria

TABLE 17
Laboratory - Hematology: White Blood Cells Standard Units

| | | | | | Abs | Abs | Abs | Abs | Abs | |
|---|---|---|---|---|---|---|---|---|---|---|
| | | | Study | WBC | Lymph | Mono | Neut | Basophils | Eos | Abs Bands |
| Subject | Treatment | Visit | Day | (10E9/L) | (10E9/L) | (10E9/L) | (10E9/L) | (10E9/L) | (10E9/L) | (10E9/L) |
| 501-0015 | 3.0 μg/kg | Screening | −61 | 4.6 | 2.10 | 0.30 | 2.00 | 0.08 | 0.10 | |
| | Q2W | | | | | | | | | |
| 501-0015 | 3.0 μg/kg | Day 1 | 1 | 5.0 | 2.20 | 0.30 | 2.30 | 0.08 | 0.12 | |
| | Q2W | | | | | | | | | |
| 501-0015 | 3.0 μg/kg | Day 8 | 8 | 4.0 | 1.70 | 0.30 | 1.80 (L1) | 0.07 | 0.11 | |
| | Q2W | | | | | | | | | |
| 501-0015 | 3.0 μg/kg | Day 15 | 15 | 5.3 | 2.40 | 0.30 | 2.40 | 0.09 | 0.19 | |
| | Q2W | | | | | | | | | |
| 501-0015 | 3.0 μg/kg | Day 22 | 22 | 5.0 | 2.00 | 0.40 | 2.40 | 0.05 | 0.07 | |
| | Q2W | | | | | | | | | |
| 501-0015 | 3.0 μg/kg | Day 29 | 31 | 4.9 | 2.20 | 0.30 | 2.20 | 0.06 | 0.14 | |
| | Q2W | | | | | | | | | |
| 501-0015 | 3.0 μg/kg | Day 59 | 66 | 4.9 | 2.20 | 0.40 | 2.20 | 0.05 | 0.11 | |
| | Q2W | | | | | | | | | |
| 501-0017 | 3.0 μg/kg | Screening | −33 | 8.8 | 3.40 | 0.50 | 4.70 | 0.06 | 0.13 | |
| | Q2W | | | | | | | | | |
| 501-0017 | 3.0 μg/kg | Day 1 | 1 | 10.1 (H) | 4.00 | 0.40 | 5.50 | 0.09 | 0.13 | |
| | Q2W | | | | | | | | | |
| 501-0017 | 3.0 μg/kg | Day 8 | 8 | 10.0 (H) | 4.10 | 0.50 | 5.10 | 0.13 | 0.13 | |
| | Q2W | | | | | | | | | |
| 501-0017 | 3.0 μg/kg | Day 15 | 15 | 10.2 (H) | 3.30 | 0.50 | 6.10 | 0.06 | 0.19 | |
| | Q2W | | | | | | | | | |
| 501-0017 | 3.0 μg/kg | Day 22 | 22 | 9.2 | 3.50 | 0.60 | 5.00 | 0.08 | 0.11 | |
| | Q2W | | | | | | | | | |
| 501-0017 | 3.0 μg/kg | Day 29 | 29 | 9.5 | 3.50 | 0.60 | 5.20 | 0.05 | 0.12 | |
| | Q2W | | | | | | | | | |
| 501-0017 | 3.0 μg/kg | Day 59 | 57 | 9.9 (H) | 3.50 | 0.60 | 5.60 | 0.05 | 0.11 | |
| | Q2W | | | | | | | | | |
| 501-0021 | 3.0 μg/kg | Screening | −26 | 6.3 | 1.90 | 0.60 | 3.50 | 0.06 | 0.22 | |
| | Q2W | | | | | | | | | |
| 501-0021 | 3.0 μg/kg | Day 1 | 1 | 7.9 | 2.20 | 0.50 | 4.70 | 0.06 | 0.33 | |
| | Q2W | | | | | | | | | |
| 501-0021 | 3.0 μg/kg | Day 8 | 8 | 5.8 | 1.80 | 0.40 | 3.30 | 0.05 | 0.17 | |
| | Q2W | | | | | | | | | |
| 501-0021 | 3.0 μg/kg | Day 15 | 15 | 5.2 | 1.80 | 0.40 | 2.80 | 0.06 | 0.23 | |
| | Q2W | | | | | | | | | |
| 501-0021 | 3.0 μg/kg | Day 22 | 22 | 6.1 | 2.10 | 0.50 | 3.40 | 0.05 | 0.07 | |
| | Q2W | | | | | | | | | |
| 501-0021 | 3.0 μg/kg | Day 29 | 31 | 5.9 | 1.80 | 0.50 | 3.10 | 0.05 | 0.34 | |
| | Q2W | | | | | | | | | |
| 501-0021 | 3.0 μg/kg | Day 59 | 59 | 6.4 | 1.70 | 0.50 | 3.90 | 0.04 | 0.24 | |
| | Q2W | | | | | | | | | |
| 502-0001 | 1.5 μg/kg | Screening | −26 | 7.1 | 2.91 | 0.64 | 3.41 | 0.07 | 0.07 | |
| | Q2W | | | | | | | | | |
| 502-0001 | 1.5 μg/kg | Day 1 | 1 | 7.8 | 2.57 | 0.70 | 4.45 | 0.00 | 0.08 | |
| | Q2W | | | | | | | | | |
| 502-0001 | 1.5 μg/kg | Day 8 | 8 | 7.7 | 2.62 | 0.46 | 4.47 | 0.00 | 0.15 | |
| | Q2W | | | | | | | | | |
| 502-0001 | 1.5 μg/kg | Day 15 | 15 | 6.9 | 2.55 | 0.55 | 3.73 | 0.00 | 0.07 | |
| | Q2W | | | | | | | | | |
| 502-0001 | 1.5 μg/kg | Day 22 | 22 | 5.2 | 2.29 | 0.47 | 2.34 | 0.00 | 0.10 | |
| | Q2W | | | | | | | | | |
| 502-0001 | 1.5 μg/kg | Day 29 | 29 | 5.9 | 2.24 | 0.53 | 3.07 | 0.00 | 0.06 | |
| | Q2W | | | | | | | | | |
| 502-0001 | 1.5 μg/kg | Day 59 | 59 | 5.1 | 1.84 | 0.46 | 2.70 | 0.00 | 0.10 | |
| | Q2W | | | | | | | | | |
| 502-0003 | 1.5 μg/kg | Screening | −25 | 6.6 | 2.05 | 0.53 | 3.89 | 0.00 | 0.13 | |
| | Q2W | | | | | | | | | |
| 502-0003 | 1.5 μg/kg | Day 1 | 1 | 5.3 | 1.96 | 0.58 | 2.60 | 0.00 | 0.16 | |
| | Q2W | | | | | | | | | |
| 502-0003 | 1.5 μg/kg | Day 8 | 8 | 4.6 | 1.66 | 0.46 | 2.35 | 0.00 | 0.14 | |
| | Q2W | | | | | | | | | |
| 502-0003 | 1.5 μg/kg | Day 15 | 15 | 7.2 | 1.94 | 0.79 | 4.32 | 0.00 | 0.14 | |
| | Q2W | | | | | | | | | |
| 502-0003 | 1.5 μg/kg | Day 22 | 22 | 5.1 | 2.04 | 0.46 | 2.40 | 0.05 | 0.15 | |
| | Q2W | | | | | | | | | |
| 502-0003 | 1.5 μg/kg | Day 29 | 29 | 4.7 | 1.32 | 0.52 | 2.73 | 0.00 | 0.14 | |
| | Q2W | | | | | | | | | |
| 502-0003 | 1.5 μg/kg | Day 59 | 59 | 6.6 | 1.39 | 0.46 | 4.55 | 0.00 | 0.20 | |
| | Q2W | | | | | | | | | |
| 502-0008 | 1.5 μg/kg | Screening | −22 | 9.5 | 2.70 | 0.60 | 6.10 | 0.10 | 0.10 | |
| | Q2W | | | | | | | | | |
| 502-0008 | 1.5 μg/kg | Day 1 | 1 | 7.5 | 1.73 | 0.53 | 5.03 | 0.00 | 0.23 | |
| | Q2W | | | | | | | | | |
| 502-0008 | 1.5 μg/kg | Day 8 | 8 | 8.1 | 1.86 | 0.49 | 5.51 | 0.00 | 0.24 | |
| | Q2W | | | | | | | | | |
| 502-0008 | 1.5 μg/kg | Day 15 | 15 | 9.6 | 2.50 | 0.67 | 6.14 | 0.10 | 0.19 | |
| | Q2W | | | | | | | | | |
| 502-0008 | 1.5 μg/kg | Day 22 | 22 | 8.2 | 1.64 | 0.49 | 5.82 | 0.08 | 0.16 | |
| | Q2W | | | | | | | | | |
| 502-0008 | 1.5 μg/kg | Day 29 | 29 | 9.5 | 1.90 | 0.48 | 6.94 | 0.00 | 0.19 | |
| | Q2W | | | | | | | | | |
| 502-0008 | 1.5 μg/kg | Day 59 | 59 | 10.3 | 2.37 | 0.41 | 7.42 | 0.00 | 0.10 | |
| | Q2W | | | | | | | | | |
| 502-0009 | 1.5 μg/kg | Screening | −18 | 5.8 | 1.51 | 0.52 | 3.54 | 0.06 | 0.17 | |
| | Q2W | | | | | | | | | |
| 502-0009 | 1.5 μg/kg | Day 1 | 1 | 5.4 | 1.03 | 0.43 | 3.78 | 0.00 | 0.16 | |
| | Q2W | | | | | | | | | |
| 502-0009 | 1.5 μg/kg | Day 8 | 8 | 6.4 | 1.22 | 0.45 | 4.48 | 0.06 | 0.19 | |
| | Q2W | | | | | | | | | |
| 502-0009 | 1.5 μg/kg | Day 15 | 16 | 5.8 | 1.39 | 0.52 | 3.65 | 0.06 | 0.17 | |
| | Q2W | | | | | | | | | |
| 502-0009 | 1.5 μg/kg | Day 22 | 22 | 5.2 | 1.25 | 0.42 | 3.33 | 0.05 | 0.16 | |
| | Q2W | | | | | | | | | |
| 502-0009 | 1.5 μg/kg | Day 29 | 29 | 5.3 | 0.95 | 0.42 | 3.76 | 0.05 | 0.11 | |
| | Q2W | | | | | | | | | |
| 502-0009 | 1.5 μg/kg | Day 59 | 59 | 6.4 | 1.54 | 0.58 | 4.03 | 0.06 | 0.19 | |
| | Q2W | | | | | | | | | |
| 502-0012 | 1.5 μg/kg | Screening | −20 | 5.7 | 1.31 | 0.51 | 3.76 | 0.00 | 0.11 | |
| | Q2W | | | | | | | | | |
| 502-0012 | 1.5 μg/kg | Day 1 | 1 | 5.3 | 1.64 | 0.53 | 2.86 | 0.05 | 0.21 | |
| | Q2W | | | | | | | | | |
| 502-0012 | 1.5 μg/kg | Day 8 | 8 | 6.7 | 1.41 | 0.54 | 4.62 | 0.00 | 0.13 | |
| | Q2W | | | | | | | | | |
| 502-0012 | 1.5 μg/kg | Day 15 | 15 | 5.5 | 1.38 | 0.44 | 3.47 | 0.06 | 0.17 | |
| | Q2W | | | | | | | | | |
| 502-0012 | 1.5 μg/kg | Day 22 | 22 | 6.0 | 1.68 | 0.84 | 3.24 | 0.06 | 0.18 | |
| | Q2W | | | | | | | | | |
| 502-0012 | 1.5 μg/kg | Day 29 | 29 | 6.2 | 1.43 | 0.68 | 3.91 | 0.00 | 0.19 | |
| | Q2W | | | | | | | | | |
| 502-0012 | 1.5 μg/kg | Day 59 | 59 | 6.9 | 1.38 | 0.62 | 4.69 | 0.07 | 0.14 | |
| | Q2W | | | | | | | | | |
| 502-0013 | 3.0 μg/kg | Screening | −61 | 6.9 | 2.55 | 0.48 | 3.73 | 0.00 | 0.14 | |
| | Q2W | | | | | | | | | |
| 502-0013 | 3.0 μg/kg | Day 1 | 1 | 5.9 | 2.07 | 0.35 | 3.30 | 0.06 | 0.12 | |
| | Q2W | | | | | | | | | |
| 502-0013 | 3.0 μg/kg | Day 8 | 8 | 6.1 | 2.38 | 0.37 | 3.17 | 0.00 | 0.18 | |
| | Q2W | | | | | | | | | |
| 502-0013 | 3.0 μg/kg | Day 15 | 14 | 5.9 | 2.36 | 0.41 | 2.95 | 0.00 | 0.18 | |
| | Q2W | | | | | | | | | |
| 502-0013 | 3.0 μg/kg | Day 22 | 22 | 6.2 | 2.29 | 0.25 | 3.47 | 0.00 | 0.19 | |
| | Q2W | | | | | | | | | |
| 502-0013 | 3.0 μg/kg | Day 29 | 29 | 5.9 | 2.07 | 0.35 | 3.30 | 0.06 | 0.12 | |
| | Q2W | | | | | | | | | |
| 502-0013 | 3.0 μg/kg | Day 59 | 59 | 6.8 | 2.24 | 0.41 | 3.94 | 0.00 | 0.20 | |
| | Q2W | | | | | | | | | |
| 502-0019 | 3.0 μg/kg | Screening | −28 | 9.4 | 2.16 | 0.47 | 6.58 | 0.09 | 0.09 | |
| | Q2W | | | | | | | | | |
| 502-0019 | 3.0 μg/kg | Day 1 | 1 | 8.5 | 2.21 | 0.43 | 5.61 | 0.00 | 0.26 | |
| | Q2W | | | | | | | | | |
| 502-0019 | 3.0 μg/kg | Day 8 | 8 | 7.5 | 2.18 | 0.38 | 4.80 | 0.00 | 0.15 | |
| | Q2W | | | | | | | | | |
| 502-0019 | 3.0 μg/kg | Day 15 | 14 | 8.5 | 2.55 | 0.51 | 5.19 | 0.00 | 0.26 | |
| | Q2W | | | | | | | | | |
| 502-0019 | 3.0 μg/kg | Day 22 | 22 | 8.6 | 2.32 | 0.34 | 5.76 | 0.00 | 0.17 | |
| | Q2W | | | | | | | | | |
| 502-0019 | 3.0 μg/kg | Day 29 | 29 | 9.7 | 2.52 | 0.68 | 6.11 | 0.00 | 0.39 | |
| | Q2W | | | | | | | | | |
| 502-0019 | 3.0 μg/kg | Day 59 | 59 | 7.4 | 2.00 | 0.44 | 4.59 | 0.07 | 0.30 | |
| | Q2W | | | | | | | | | |
| 502-0020 | 3.0 μg/kg | Screening | −27 | 5.0 | 1.00 | 0.25 | 3.70 | 0.00 | 0.05 | |
| | Q2W | | | | | | | | | |
| 502-0020 | 3.0 μg/kg | Day 1 | 1 | 4.6 | 2.07 | 0.32 | 2.12 | 0.00 | 0.09 | |
| | Q2W | | | | | | | | | |
| 502-0020 | 3.0 μg/kg | Day 8 | 8 | 3.8 (L1) | 1.22 | 0.23 | 2.28 | 0.00 | 0.08 | |
| | Q2W | | | | | | | | | |
| 502-0020 | 3.0 μg/kg | Day 15 | 14 | 4.2 | 1.93 | 0.29 | 1.93 | 0.00 | 0.04 | |
| | Q2W | | | | | | | | | |
| 502-0020 | 3.0 μg/kg | Day 22 | 22 | 3.8 (L1) | 1.03 | 0.19 | 2.55 | 0.00 | 0.04 | |
| | Q2W | | | | | | | | | |
| 502-0020 | 3.0 μg/kg | Day 29 | 29 | 3.9 (L1) | 1.40 | 0.23 | 2.22 | 0.00 | 0.04 | |
| | Q2W | | | | | | | | | |
| 502-0020 | 3.0 μg/kg | Day 59 | 59 | 5.1 | 1.48 | 0.31 | 3.26 | 0.00 | 0.05 | |
| | Q2W | | | | | | | | | |
| 502-0023 | 1.5 μg/kg | Screening | −34 | 4.4 | 1.58 | 0.26 | 2.46 | 0.00 | 0.09 | |
| | QW | | | | | | | | | |
| 502-0023 | 1.5 μg/kg | Day 1 | 1 | 3.9 (L1) | 1.48 | 0.35 | 1.91 | 0.00 | 0.16 | |
| | QW | | | | | | | | | |
| 502-0023 | 1.5 μg/kg | Day 8 | 7 | 5.6 | 2.18 | 0.50 | 2.74 | 0.00 | 0.17 | |
| | QW | | | | | | | | | |
| 502-0023 | 1.5 μg/kg | Day 15 | 14 | 5.4 | 2.00 | 0.43 | 2.81 | 0.00 | 0.16 | |
| | QW | | | | | | | | | |
| 502-0023 | 1.5 μg/kg | Day 22 | 21 | 4.0 | 1.44 | 0.28 | 2.12 | 0.00 | 0.16 | |
| | QW | | | | | | | | | |
| 502-0023 | 1.5 μg/kg | Day 29 | 29 | 3.8 (L1) | 1.44 | 0.34 | 1.82 | 0.04 | 0.15 | |
| | QW | | | | | | | | | |
| 502-0023 | 1.5 μg/kg | Day 36 | 36 | 4.3 | 1.89 | 0.30 | 1.94 | 0.00 | 0.17 | |
| | QW | | | | | | | | | |
| 502-0023 | 1.5 μg/kg | Day 59 | 59 | 4.5 | 2.39 | 0.45 | 1.49 (L2) | 0.00 | 0.18 | |
| | QW | | | | | | | | | |
| 502-0024 | 1.5 μg/kg | Screening | −26 | 15.0 (H) | 2.10 | 0.90 | 11.70 (H) | 0.15 | 0.15 | |
| | QW | | | | | | | | | |
| 502-0024 | 1.5 μg/kg | Day 1 | 1 | 9.5 | 2.85 | 0.57 | 5.23 | 0.00 | 0.86 (H) | |
| | QW | | | | | | | | | |
| 502-0024 | 1.5 μg/kg | Day 8 | 7 | 7.5 | 2.33 | 0.38 | 4.28 | 0.00 | 0.53 (H) | |
| | QW | | | | | | | | | |
| 502-0024 | 1.5 μg/kg | Day 15 | 14 | 8.2 | 2.54 | 0.49 | 4.67 | 0.00 | 0.49 (H) | |
| | QW | | | | | | | | | |
| 502-0024 | 1.5 μg/kg | Day 22 | 21 | 7.3 | 2.48 | 0.44 | 3.58 | 0.00 | 0.80 (H) | |
| | QW | | | | | | | | | |
| 502-0024 | 1.5 μg/kg | Day 29 | 29 | 6.7 | 2.55 | 0.47 | 2.95 | 0.07 | 0.67 (H) | |
| | QW | | | | | | | | | |
| 502-0024 | 1.5 μg/kg | Day 36 | 36 | 7.7 | 3.00 | 0.39 | 3.54 | 0.08 | 0.69 (H) | |
| | QW | | | | | | | | | |
| 502-0024 | 1.5 μg/kg | Day 59 | 59 | 7.8 | 2.57 | 0.55 | 3.82 | 0.08 | 0.78 (H) | |
| | QW | | | | | | | | | |
| 503-0022 | 1.5 μg/kg | Screening | −47 | 7.0 | 1.70 | 0.70 | 4.33 | 0.05 | 0.22 | |
| | QW | | | | | | | | | |
| 503-0022 | 1.5 μg/kg | Day 1 | 1 | 6.8 | 1.43 | 0.67 | 4.47 | 0.04 | 0.18 | |
| | QW | | | | | | | | | |
| 503-0022 | 1.5 μg/kg | Day 15 | 15 | 6.9 | 1.65 | 0.70 | 4.31 | 0.03 | 0.21 | |
| | QW | | | | | | | | | |
| 503-0022 | 1.5 μg/kg | Day 22 | 22 | 6.6 | 1.49 | 0.55 | 4.28 | 0.04 | 0.24 | |
| | QW | | | | | | | | | |
| 503-0022 | 1.5 μg/kg | Day 29 | 29 | 6.3 | 1.55 | 0.56 | 3.94 | 0.04 | 0.21 | |
| | QW | | | | | | | | | |
| 503-0022 | 1.5 μg/kg | Day 36 | 38 | 6.4 | 1.45 | 0.66 | 4.06 | 0.04 | 0.18 | |
| | QW | | | | | | | | | |
| 503-0022 | 1.5 μg/kg | Day 59 | 57 | 6.5 | 1.42 | 0.59 | 4.24 | 0.05 | 0.21 | |
| | QW | | | | | | | | | |
| 505-0006 | 1.5 μg/kg | Screening | −33 | 7.2 | 2.45 | 0.65 | 3.96 | 0.00 | 0.14 | 0.00 |
| | Q2W | | | | | | | | | |
| 505-0006 | 1.5 μg/kg | Day 1 | 1 | 7.2 | 2.81 | 0.58 | 3.60 | 0.00 | 0.14 | 0.00 |
| | Q2W | | | | | | | | | |
| 505-0006 | 1.5 μg/kg | Day 8 | 8 | 6.1 | 2.75 | 0.37 | 2.87 | 0.00 | 0.12 | 0.00 |
| | Q2W | | | | | | | | | |
| 505-0006 | 1.5 μg/kg | Day 15 | 16 | 7.7 | 2.08 | 0.77 | 4.70 | 0.00 | 0.23 | 0.00 |
| | Q2W | | | | | | | | | |
| 505-0006 | 1.5 μg/kg | Day 22 | 23 | 10.4 | 3.22 | 0.62 | 6.45 | 0.00 | 0.21 | 3-4 (M) |
| | Q2W | | | | | | | | | |
| 505-0006 | 1.5 μg/kg | Day 29 | 29 | 6.2 | 2.54 | 0.37 | 3.16 | 0.06 | 0.06 | 0-1 (M) |
| | Q2W | | | | | | | | | |
| 505-0006 | 1.5 μg/kg | Day 59 | 59 | 6.2 | 2.36 | 0.68 | 3.04 | 0.00 | 0.12 | 0.00 |
| | Q2W | | | | | | | | | |
| 505-0027 | 1.5 μg/kg | Screening | −19 | 4.5 | 1.26 | 0.32 | 2.88 | 0.05 | 0.05 | 0.00 |
| | QW | | | | | | | | | |
| 505-0027 | 1.5 μg/kg | Day 1 | 1 | 3.4 (L1) | 1.05 | 0.27 | 2.04 | 0.03 | 0.03 | 0.00 |
| | QW | | | | | | | | | |
| 505-0027 | 1.5 μg/kg | Day 8 | 8 | 4.2 (L1) | 1.34 | 0.29 | 2.52 | 0.04 | 0.00 | 0.00 |
| | QW | | | | | | | | | |
| 505-0027 | 1.5 μg/kg | Day 15 | 15 | 3.9 (L1) | 1.09 | 0.27 | 2.50 | 0.04 | 0.04 | 0.00 |
| | QW | | | | | | | | | |
| 505-0027 | 1.5 μg/kg | Day 22 | 22 | 3.0 (L1) | 1.08 | 0.24 | 1.62 (L1) | 0.03 | 0.03 | 0.00 |
| | QW | | | | | | | | | |
| 505-0027 | 1.5 μg/kg | Day 29 | 29 | 4.0 (L1) | 1.20 | 0.24 | 2.48 | 0.04 | 0.00 | 0.00 |
| | QW | | | | | | | | | |
| 505-0027 | 1.5 μg/kg | Day 36 | 38 | 3.9 (L1) | 1.29 | 0.27 | 2.26 | 0.04 | 0.04 | 0.00 |
| | QW | | | | | | | | | |
| 505-0027 | 1.5 μg/kg | Day 59 | 59 | 4.1 (L1) | 0.94 (L1) | 0.25 | 2.87 | 0.04 | 0.04 | 0.00 |
| | QW | | | | | | | | | |
| 506-0032 | 1.5 μg/kg | Screening | −51 | 8.0 | 2.10 | 0.60 | 5.10 | 0.00 | 0.20 | |
| | QW | | | | | | | | | |
| 506-0032 | 1.5 μg/kg | Day 1 | 1 | 6.6 | 2.00 | 0.50 | 3.90 | 0.00 | 0.10 | |
| | QW | | | | | | | | | |
| 506-0032 | 1.5 μg/kg | Day 8 | 8 | 6.5 | 2.00 | 0.50 | 3.80 | 0.00 | 0.10 | |
| | QW | | | | | | | | | |
| 506-0032 | 1.5 μg/kg | Day 15 | 15 | 6.2 | 1.70 | 0.40 | 3.90 | 0.00 | 0.20 | |
| | QW | | | | | | | | | |
| 506-0032 | 1.5 μg/kg | Day 22 | 23 | 5.5 | 1.80 | 0.40 | 3.20 | 0.00 | 0.10 | |
| | QW | | | | | | | | | |
| 506-0032 | 1.5 μg/kg | Day 29 | 29 | 4.8 | 1.60 | 0.40 | 2.80 | 0.00 | 0.10 | |
| | QW | | | | | | | | | |
| 506-0032 | 1.5 μg/kg | Day 36 | 36 | 5.0 | 1.80 | 0.40 | 2.70 | 0.00 | 0.10 | |
| | QW | | | | | | | | | |
| 507-0028 | 1.5 μg/kg | Screening | −40 | 5.1 | 2.60 | 0.40 | 2.00 | 0.10 | 0.10 | |
| | QW | | | | | | | | | |
| 507-0028 | 1.5 μg/kg | Unscheduled | −22 | 5.7 | 2.70 | 8.10 (H) | 2.40 | 0.00 | 0.10 | |
| | QW | | | | | | | | | |
| 507-0028 | 1.5 μg/kg | Day 1 | 1 | 5.2 | 2.50 | 0.40 | 2.10 | 0.00 | 0.20 | |
| | QW | | | | | | | | | |
| 507-0028 | 1.5 μg/kg | Day 8 | 8 | 5.1 | 2.60 | 0.40 | 2.00 | 0.00 | 0.10 | 0 (U) |
| | QW | | | | | | | | | |
| 507-0028 | 1.5 μg/kg | Day 15 | 15 | 6.4 | 2.80 | 0.60 (H) | 3.00 | 0.00 | 0.10 | 0 (U) |
| | QW | | | | | | | | | |
| 507-0028 | 1.5 μg/kg | Day 22 | 22 | 5.2 | 2.30 | 0.40 | 2.40 | 0.00 | 0.10 | |
| | QW | | | | | | | | | |
| 507-0028 | 1.5 μg/kg | Day 29 | 30 | 6.0 | 2.70 | 0.40 | 2.80 | 0.00 | 0.10 | |
| | QW | | | | | | | | | |
| 507-0028 | 1.5 μg/kg | Day 36 | 37 | 5.5 | 2.50 | 0.50 | 2.40 | 0.00 | 0.10 | |
| | QW | | | | | | | | | |

Note:
L = below lower limit of reference range,
H = above upper limit of reference range,
M = missing reference range,
U = Collected units are unknown
If Flag = M or U then the collected result was not converted to standard result. The collected result is displayed.
Numbers following the L/H flags indicate grades based on CTCAE grading criteria Error! Bookmark not defined.

TABLE 18
Laboratory - Coagulation Standard Units

| | | | Study | PT | aPTT | | Fibrinogen |
|---|---|---|---|---|---|---|---|
| Subject | Treatment | Visit | Day | (sec) | (sec) | INR | (g/L) |
| 501-0015 | 3.0 μg/kg Q2W | Screening | −61 | 10.9 (L) | 36.6 (H1) | 1.0 | 3.34 |
| 501-0015 | 3.0 μg/kg Q2W | Day 1 | 1 | 10.6 (L) | | 0.9 | 3.82 |
| 501-0015 | 3.0 μg/kg Q2W | Day 8 | 8 | 10.3 (L) | 35.8 (H1) | 0.9 | 3.30 |
| 501-0015 | 3.0 μg/kg Q2W | Day 15 | 15 | 10.1 (L) | 35.0 (H1) | 0.9 | 4.06 |
| 501-0015 | 3.0 μg/kg Q2W | Day 22 | 22 | 10.7 (L) | 35.1 (H1) | 1.0 | 3.33 |
| 501-0015 | 3.0 μg/kg Q2W | Day 29 | 31 | 10.7 (L) | 36.7 (H1) | 1.0 | 4.06 |
| 501-0015 | 3.0 μg/kg Q2W | Day 59 | 66 | 10.8 (L) | 35.1 (H1) | 1.0 | 3.60 |
| 501-0017 | 3.0 μg/kg Q2W | Screening | −33 | 10.2 (L) | 35.2 (H1) | 0.9 | 4.85 (H) |
| 501-0017 | 3.0 μg/kg Q2W | Day 1 | 1 | 9.9 (L) | 33.3 (H1) | 0.9 | 4.72 (H) |
| 501-0017 | 3.0 μg/kg Q2W | Day 8 | 8 | 10.0 (L) | 32.4 (H1) | 0.9 | 4.40 (H) |
| 501-0017 | 3.0 μg/kg Q2W | Day 15 | 15 | 10.1 (L) | 35.6 (H1) | 0.9 | 4.78 (H) |
| 501-0017 | 3.0 μg/kg Q2W | Day 22 | 22 | 10.0 (L) | 32.1 | 0.9 | 4.40 (H) |
| 501-0017 | 3.0 μg/kg Q2W | Day 29 | 29 | 10.1 (L) | 35.3 (H1) | 0.9 | 5.34 (H) |
| 501-0017 | 3.0 μg/kg Q2W | Unscheduled | 43 | 10.2 (L) | | 0.9 | |
| 501-0017 | 3.0 μg/kg Q2W | Day 59 | 57 | 10.0 (L) | 33.7 (H1) | 0.9 | 4.94 (H) |
| 501-0021 | 3.0 μg/kg Q2W | Screening | −26 | 10.7 (L) | 34.0 (H1) | 1.0 | 3.08 |
| 501-0021 | 3.0 μg/kg Q2W | Day 1 | 1 | 10.8 (L) | 35.0 (H1) | 1.0 | 2.77 |
| 501-0021 | 3.0 μg/kg Q2W | Day 8 | 8 | 11.4 | 34.8 (H1) | 1.0 | 2.90 |
| 501-0021 | 3.0 μg/kg Q2W | Day 15 | 15 | 10.9 (L) | 37.7 (H1) | 1.0 | 2.98 |
| 501-0021 | 3.0 μg/kg Q2W | Day 22 | 22 | 10.8 (L) | 34.6 (H1) | 1.0 | 2.69 |
| 501-0021 | 3.0 μg/kg Q2W | Day 29 | 31 | 11.5 | 34.5 (H1) | 1.0 | 2.92 |
| 501-0021 | 3.0 μg/kg Q2W | Day 59 | 59 | 11.4 | 35.7 (H1) | 1.0 | 3.07 |
| 502-0001 | 1.5 μg/kg Q2W | Screening | −26 | 10.6 | 27.0 | 1.0 (L) | 2.40 |
| 502-0001 | 1.5 μg/kg Q2W | Day 1 | 1 | 10.0 | 26.0 | 1.0 (L) | 1.58 (L1) |
| 502-0001 | 1.5 μg/kg Q2W | Day 8 | 8 | 9.9 | 27.0 | 0.9 (L) | 2.74 |
| 502-0001 | 1.5 μg/kg Q2W | Day 15 | 15 | 11.0 | 29.0 | 1.1 (L) | 2.61 |
| 502-0001 | 1.5 μg/kg Q2W | Day 22 | 22 | 10.4 | 28.0 | 1.0 (L) | |
| 502-0001 | 1.5 μg/kg Q2W | Unscheduled | 25 | | | | 2.26 |
| 502-0001 | 1.5 μg/kg Q2W | Day 29 | 29 | 10.8 | 29.0 | 1.1 (L) | 3.10 |
| 502-0001 | 1.5 μg/kg Q2W | Unscheduled | 47 | | | | 2.50 |
| 502-0001 | 1.5 μg/kg Q2W | Day 59 | 59 | 10.4 | 27.0 | 1.0 (L) | 1.87 |
| 502-0003 | 1.5 μg/kg Q2W | Screening | −25 | 10.6 | 27.0 | 1.0 (L) | 3.03 |
| 502-0003 | 1.5 μg/kg Q2W | Day 1 | 1 | 10.6 | 28.0 | 1.0 (L) | 2.74 |
| 502-0003 | 1.5 μg/kg Q2W | Day 8 | 8 | 10.5 | 27.0 | 1.0 (L) | 2.41 |
| 502-0003 | 1.5 μg/kg Q2W | Day 15 | 15 | 10.6 | 28.0 | 1.0 (L) | 2.43 |
| 502-0003 | 1.5 μg/kg Q2W | Day 22 | 22 | 10.4 | 27.0 | 1.0 (L) | 3.16 |
| 502-0003 | 1.5 μg/kg Q2W | Day 29 | 29 | 10.2 | 27.0 | 1.0 (L) | 3.02 |
| 502-0003 | 1.5 μg/kg Q2W | Day 59 | 59 | 10.4 | 27.0 | 1.0 (L) | |
| 502-0003 | 1.5 μg/kg Q2W | Unscheduled | 64 | | | | 2.50 |
| 502-0008 | 1.5 μg/kg Q2W | Screening | −22 | 10.5 | 47.0 (H1) | 1.0 (L) | 2.26 |
| 502-0008 | 1.5 μg/kg Q2W | Day 1 | 1 | 9.9 | 28.0 | 1.0 (L) | 2.65 |
| 502-0008 | 1.5 μg/kg Q2W | Day 8 | 8 | 9.9 | 28.0 | 1.0 (L) | 2.00 |
| 502-0008 | 1.5 μg/kg Q2W | Day 15 | 15 | 9.9 | 27.0 | 0.9 (L) | 2.40 |
| 502-0008 | 1.5 μg/kg Q2W | Day 22 | 22 | 9.7 | 28.0 | 0.9 (L) | 1.57 (L1) |
| 502-0008 | 1.5 μg/kg Q2W | Day 29 | 29 | 9.8 | 27.0 | 1.0 (L) | 1.95 |
| 502-0008 | 1.5 μg/kg Q2W | Day 59 | 59 | 10.3 | 28.0 | 1.0 (L) | 2.26 |
| 502-0009 | 1.5 μg/kg Q2W | Screening | −18 | 10.7 | 25.0 | 1.0 (L) | 3.14 |
| 502-0009 | 1.5 μg/kg Q2W | Day 1 | 1 | 10.2 | 26.0 | 1.0 (L) | 3.10 |
| 502-0009 | 1.5 μg/kg Q2W | Day 8 | 8 | 10.2 | 27.0 | 1.0 (L) | 2.46 |
| 502-0009 | 1.5 μg/kg Q2W | Day 15 | 16 | 10.4 | 27.0 | 1.0 (L) | 1.93 |
| 502-0009 | 1.5 μg/kg Q2W | Day 22 | 22 | 10.3 | 27.0 | 1.0 (L) | 2.64 |
| 502-0009 | 1.5 μg/kg Q2W | Day 29 | 29 | 10.3 | 28.0 | 1.0 (L) | 2.50 |
| 502-0009 | 1.5 μg/kg Q2W | Day 59 | 59 | 10.4 | 27.0 | 1.0 (L) | 2.29 |
| 502-0012 | 1.5 μg/kg Q2W | Screening | −20 | 9.6 | 27.0 | 0.9 (L) | |
| 502-0012 | 1.5 μg/kg Q2W | Unscheduled | −12 | | | | 2.72 |
| 502-0012 | 1.5 μg/kg Q2W | Day 1 | 1 | 9.7 | 26.0 | 0.9 (L) | 2.37 |
| 502-0012 | 1.5 μg/kg Q2W | Day 8 | 8 | 9.4 | 29.0 | 0.9 (L) | 1.56 (L1) |
| 502-0012 | 1.5 μg/kg Q2W | Day 15 | 15 | 9.4 | 26.0 | 0.9 (L) | 2.14 |
| 502-0012 | 1.5 μg/kg Q2W | Day 22 | 22 | 9.4 | 28.0 | 0.9 (L) | 2.12 |
| 502-0012 | 1.5 μg/kg Q2W | Day 29 | 29 | 9.7 | 27.0 | 0.9 (L) | 3.68 (H) |
| 502-0012 | 1.5 μg/kg Q2W | Day 59 | 59 | 9.4 | 27.0 | 0.9 (L) | 2.53 |
| 502-0013 | 3.0 μg/kg Q2W | Screening | −61 | 10.4 | 30.0 | 1.0 (L) | 2.99 |
| 502-0013 | 3.0 μg/kg Q2W | Day 1 | 1 | 10.3 | 30.0 | 1.0 (L) | 2.05 |
| 502-0013 | 3.0 μg/kg Q2W | Day 8 | 8 | 10.0 | 30.0 | 1.0 (L) | 1.27 (L2) |
| 502-0013 | 3.0 μg/kg Q2W | Day 15 | 14 | 9.5 | 28.0 | 1.0 (L) | 1.88 |
| 502-0013 | 3.0 μg/kg Q2W | Day 22 | 22 | 10.2 | 32.0 | 1.0 (L) | 2.63 |
| 502-0013 | 3.0 μg/kg Q2W | Day 29 | 29 | 10.1 | 31.0 | 1.0 (L) | 2.35 |
| 502-0013 | 3.0 μg/kg Q2W | Day 59 | 59 | 10.6 | 33.0 | 1.1 (L) | 2.13 |
| 502-0019 | 3.0 μg/kg Q2W | Screening | −28 | 10.2 | 32.0 | 1.0 (L) | 3.51 (H) |
| 502-0019 | 3.0 μg/kg Q2W | Day 1 | 1 | 10.1 | 32.0 | 1.0 (L) | 3.01 |
| 502-0019 | 3.0 μg/kg Q2W | Day 8 | 8 | 9.9 | 30.0 | 1.0 (L) | 1.84 |
| 502-0019 | 3.0 μg/kg Q2W | Day 15 | 14 | 9.5 | 28.0 | 1.0 (L) | 3.31 |
| 502-0019 | 3.0 μg/kg Q2W | Day 22 | 22 | 10.2 | 31.0 | 1.0 (L) | 1.94 |
| 502-0019 | 3.0 μg/kg Q2W | Day 29 | 29 | 10.1 | 30.0 | 1.0 (L) | |
| 502-0019 | 3.0 μg/kg Q2W | Unscheduled | 34 | | | | 3.18 |
| 502-0019 | 3.0 μg/kg Q2W | Day 59 | 59 | 9.9 | 30.0 | 1.0 (L) | 3.83 (H) |
| 502-0020 | 3.0 μg/kg Q2W | Screening | −27 | 9.8 | 29.0 | 0.9 (L) | 2.18 |
| 502-0020 | 3.0 μg/kg Q2W | Day 1 | 1 | 9.7 | 29.0 | 0.9 (L) | 2.95 |
| 502-0020 | 3.0 μg/kg Q2W | Day 8 | 8 | 9.9 | 31.0 | 1.0 (L) | 1.89 |
| 502-0020 | 3.0 μg/kg Q2W | Day 15 | 14 | 9.1 | 29.0 | 1.0 (L) | 2.97 |
| 502-0020 | 3.0 μg/kg Q2W | Day 22 | 22 | 9.7 | 32.0 | 0.9 (L) | 3.03 |
| 502-0020 | 3.0 μg/kg Q2W | Day 29 | 29 | 9.7 | 30.0 | 0.9 (L) | 3.60 (H) |
| 502-0020 | 3.0 μg/kg Q2W | Day 59 | 59 | 10.3 | 31.0 | 1.0 (L) | 3.30 |
| 502-0023 | 1.5 μg/kg QW | Screening | −34 | 10.3 | 28.0 | 1.0 (L) | 2.02 |
| 502-0023 | 1.5 μg/kg QW | Day 1 | 1 | 10.4 | 31.0 | 1.0 (L) | |
| 502-0023 | 1.5 μg/kg QW | Day 8 | 7 | 9.5 | 30.0 | 1.0 (L) | 2.24 |
| 502-0023 | 1.5 μg/kg QW | Day 15 | 14 | 9.8 | 31.0 | 1.0 (L) | 3.63 (H) |
| 502-0023 | 1.5 μg/kg QW | Day 22 | 21 | 9.4 | 28.0 | 1.0 (L) | 2.02 |
| 502-0023 | 1.5 μg/kg QW | Day 29 | 29 | 10.5 | 32.0 | 1.1 (L) | 2.07 |
| 502-0023 | 1.5 μg/kg QW | Day 36 | 36 | 10.5 | 30.0 | 1.1 (L) | 2.18 |
| 502-0023 | 1.5 μg/kg QW | Day 59 | 59 | 10.5 | 30.0 | 1.1 (L) | 2.18 |
| 502-0024 | 1.5 μg/kg QW | Screening | −26 | 10.9 | 30.0 | 1.1 (L) | 4.55 (H) |
| 502-0024 | 1.5 μg/kg QW | Day 1 | 1 | 10.8 | 28.0 | 1.0 (L) | 1.60 (L1) |
| 502-0024 | 1.5 μg/kg QW | Day 8 | 7 | 10.0 | 26.0 | 1.1 (L) | 1.73 (L1) |
| 502-0024 | 1.5 μg/kg QW | Day 15 | 14 | 9.9 | 28.0 | 1.0 (L) | 2.88 |
| 502-0024 | 1.5 μg/kg QW | Day 22 | 21 | 9.8 | 29.0 | 1.0 (L) | 3.02 |
| 502-0024 | 1.5 μg/kg QW | Day 29 | 29 | 10.9 | 30.0 | 1.1 (L) | 2.02 |
| 502-0024 | 1.5 μg/kg QW | Day 36 | 36 | 11.0 | 29.0 | 1.1 (L) | 3.23 |
| 502-0024 | 1.5 μg/kg QW | Day 59 | 59 | 11.5 | 29.0 | 1.2 (L) | 2.78 |
| 503-0022 | 1.5 μg/kg QW | Screening | −47 | 13.6 | 26.3 | 1.0 (L) | 3.66 |
| 503-0022 | 1.5 μg/kg QW | Day 1 | 1 | 12.9 | 26.9 | 1.0 (L) | 3.76 |
| 503-0022 | 1.5 μg/kg QW | Day 15 | 15 | 13.3 | 27.4 | 1.0 (L) | 3.66 |
| 503-0022 | 1.5 μg/kg QW | Day 22 | 22 | 13.8 | 29.1 | 1.1 (L) | 3.32 |
| 503-0022 | 1.5 μg/kg QW | Day 29 | 29 | 12.7 | 40.3 (H1) | 1.0 (L) | 3.62 |
| 503-0022 | 1.5 μg/kg QW | Day 36 | 38 | 13.7 | 28.4 | 1.0 (L) | 3.89 |
| 503-0022 | 1.5 μg/kg QW | Day 59 | 57 | 13.5 | 25.9 | 1.0 (L) | |
| 505-0006 | 1.5 μg/kg Q2W | Screening | −33 | 13.7 | | 1.1 | |
| 505-0006 | 1.5 μg/kg Q2W | Unscheduled | −20 | 13.1 | 32.5 | 1.0 | 3.00 |
| 505-0006 | 1.5 μg/kg Q2W | Day 1 | 1 | 13.4 | | 1.0 | 3.15 |
| 505-0006 | 1.5 μg/kg Q2W | Day 8 | 8 | 13.5 | 34.9 | 1.0 | 2.60 |
| 505-0006 | 1.5 μg/kg Q2W | Day 15 | 16 | 13.7 | 36.0 | 1.1 | 4.43 |
| 505-0006 | 1.5 μg/kg Q2W | Day 22 | 23 | 13.2 | 31.5 | 1.0 | 4.63 (H) |
| 505-0006 | 1.5 μg/kg Q2W | Day 29 | 29 | 13.4 | 32.0 | 1.0 | 3.84 |
| 505-0006 | 1.5 μg/kg Q2W | Day 59 | 59 | 13.5 | 33.2 | 1.0 | 3.14 |
| 505-0027 | 1.5 μg/kg QW | Screening | −14 | 14.0 | 34.7 | 1.1 | 4.05 |
| 505-0027 | 1.5 μg/kg QW | Day 1 | 1 | 14.0 | 34.1 | 1.1 | 3.41 |
| 505-0027 | 1.5 μg/kg QW | Day 8 | 8 | 13.8 | 33.2 | 1.1 | 3.64 |
| 505-0027 | 1.5 μg/kg QW | Day 15 | 15 | 14.2 | 34.2 | 1.1 | 3.20 |
| 505-0027 | 1.5 μg/kg QW | Day 22 | 22 | 13.4 | 33.5 | 1.0 | 3.64 |
| 505-0027 | 1.5 μg/kg QW | Day 29 | 29 | 14.1 | 33.2 | 1.1 | 3.90 |
| 505-0027 | 1.5 μg/kg QW | Day 36 | 38 | 13.9 | 34.0 | 1.1 | 3.85 |
| 505-0027 | 1.5 μg/kg QW | Day 59 | 59 | 13.6 | 34.6 | 1.1 | 3.60 |
| 506-0032 | 1.5 μg/kg QW | Screening | −51 | 11.8 (L) | 32.0 (U) | 1.0 | 3.93 |
| 506-0032 | 1.5 μg/kg QW | Day 1 | 1 | 12.1 (L) | 34.1 (U) | 1.0 | 3.89 |
| 506-0032 | 1.5 μg/kg QW | Day 8 | 8 | 12.1 (L) | 32.8 (U) | 1.0 | 3.67 |
| 506-0032 | 1.5 μg/kg QW | Day 15 | 15 | 11.3 (L) | 34.0 (U) | 1.0 | 3.75 |
| 506-0032 | 1.5 μg/kg QW | Day 29 | 29 | 12.0 (L) | 33.8 (U) | 1.0 | 2.95 |
| 506-0032 | 1.5 μg/kg QW | Day 36 | 36 | 11.3 (L) | 35.0 (U) | 1.0 | 3.61 |
| 507-0028 | 1.5 μg/kg QW | Screening | −40 | 12.6 | 31.0 | 0.9 (M) | 3.27 |
| 507-0028 | 1.5 μg/kg QW | Day 1 | 1 | 13.6 | 29.0 | 1.0 (M) | 2.86 |
| 507-0028 | 1.5 μg/kg QW | Day 8 | 8 | 12.7 | 32.0 | 0.9 (M) | 3.00 |
| 507-0028 | 1.5 μg/kg QW | Day 15 | 15 | 12.6 | 32.0 | 0.9 (M) | 3.09 |
| 507-0028 | 1.5 μg/kg QW | Day 22 | 22 | 12.1 (L) | 32.0 | 0.9 (M) | 3.18 |
| 507-0028 | 1.5 μg/kg QW | Day 29 | 30 | 2.9 (L) | 34.0 | 0.9 (M) | 3.49 |
| 507-0028 | 1.5 μg/kg QW | Day 36 | 37 | 12.3 (L) | 31.0 | 0.9 (M) | 3.70 |

Note:
L = below lower limit of reference range,
H = above upper limit of reference range,
M = missing reference range,
U = Collected units are unknown
If Flag = M or U then the collected result was not converted to standard result. The collected result is displayed.
Numbers following the L/H flags indicate grades based on CTCAE grading criteria

The complete disclosures of the patents, patent documents, and publications cited herein are incorporated by reference in their entirety as if each were individually incorporated. Various modifications and alterations to this invention will become apparent to those skilled in the art without departing from the scope and spirit of this invention. It should be understood that this invention is not intended to be unduly limited by the illustrative embodiments and examples set forth herein and that such examples and embodiments are presented by way of example only with the scope of the invention intended to be limited only by the claims set forth herein as follows.

What is claimed is:

1. A method of treating a human patient infected or at risk of infection with the hepatitis C virus comprising administering to the human patient a therapeutically effective amount of a Pegylated Type III Interferon.

2. A method of treating a human patient infected or at risk of infection with the hepatitis C virus comprising administering to the human patient a therapeutically effective amount of a pharmaceutical formulation comprising a Pegylated Type III Interferon and a pharmaceutically acceptable vehicle.

3. The method of claims 1 and 2 wherein the Pegylated Type III Interferon is administered to the patient according to a dosing schedule selected from the group consisting of one dose per week, two doses per week, three doses per week, one dose every other day, one dose every three days, and one dose every two weeks.

4. The method of claims 1 and 2 wherein the Type III Interferon is selected from the group consisting of an IL-28A polypeptide, an IL-28B polypeptide, and an IL-29 polypeptide.

5. The method of claim 4 wherein the IL-28A polypeptide is selected from the group consisting of SEQ ID NOs:2, 4, 6, 8, 10 and 12.

6. The method of claim 4 wherein the IL-28B polypeptide is selected from the group consisting of SEQ ID NOs:14, 16, 18, 20, 22, 24, 26, 28, 30 and 32.

7. The method of claim 4 wherein the IL-29 polypeptide is selected from the group consisting of SEQ ID NOs:34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 115, 117, 119, 121 and 123.

8. The method of claims 1 and 2 wherein the Pegylated Type III Interferon or pharmaceutical formulation is administered parenterally.

9. The method of claim 8 wherein the Pegylated Type III Interferon is administered by injection or infusion.

10. The method of claim 8 wherein the Pegylated Type III Interferon or pharmaceutical formulation is administered intravenously, intramuscularly, subcutaneously, intradermally, or intraperitoneally.

11. The method of claims 1 and 2 wherein the therapeutically effective amount of Pegylated Type III Interferon or pharmaceutical formulation is administered to the patient in a dose amount selected from the group consisting of less than 0.5 μg/kg, 0.5 to 1.0 μg/kg, 1.0 to 1.5 μg/kg, 1.5 to 2.0 μg/kg, 2.0 to 2.5 μg/kg, 2.5 to 3.0 μg/kg, 3.0 to 3.5 μg/kg, 3.5 to 4.0 μg/kg, 4.0 to 4.5 μg/kg, 4.5 to 5.0 μg/kg, 5.0 to 5.5 μg/kg, 5.5 to 6.0 μg/kg, 6.0 to 6.5 μg/kg, 6.5 to 7.0 μg/kg, 7.0 to 7.5 μg/kg, 7.5 to 8.0 μg/kg, 8.0 to 8.5 μg/kg, 8.5 to 9.0 μg/kg, 9.0 to 9.5 μg/kg, 9.5 to 10.0 μg/kg, greater than 10.0 μg/kg, fixed dose of about 60-80 μg, fixed dose of about 80-100 μg, fixed dose of about 100-120 μg, fixed dose of about 120-140 μg, fixed dose of about 140-160 μg, fixed dose of about 160-180 μg, fixed dose of about 180-200 μg, fixed dose of about 200-220 μg, fixed dose of about 220-240 μg, fixed dose of about 240-260 μg, fixed dose of about 260-280 μg, and fixed dose of about 280-300 μg.

12. The method of claims 1 and 2 wherein the patient is selected from a subpopulation of hepatitis C patients consisting of treatment naïve patients with genotype I hepatitis C; treatment naïve patients with any genotype hepatitis C; patients co-infected with the human immunodeficiency virus (HIV); patients intolerant to Pegylated Interferon Alpha, Interferon Alpha or any other Pegylated or NonPegylated Type I Interferon; patients for whom treatment with Pegylated Interferon Alpha, Interferon Alpha or any other Pegylated or NonPegylated Type I Interferon is contraindicated; patients awaiting or following liver transplant; patients with decompensated liver disease; patients who are previous non-responders to treatment with Pegylated Interferon Alpha, Interferon Alpha or any other Pegylated or NonPegylated Type I Interferon either as a single agent or in combination with ribavirin or any other anti-hepatitis C agent, including patients who were null responders, responder/relapsers, or break-through patients; patients who were non-compliant with prior treatment with Pegylated Interferon Alpha, Interferon Alpha or any other Pegylated or NonPegylated Type I Interferon either as a single agent or in combination with ribavirin or other any of the anti-hepatitis C agents; patients with any base level of hepatitis C RNA; and patients with cirrhosis.

13. The method of claims 1 and 2 wherein the duration of the treatment is less than 20 weeks, 20-24 weeks, 24-28 weeks, 28-32 weeks, 32-36 weeks, 36-40 weeks, 40-44 weeks, 44-48 weeks, 48-52 weeks, or greater than 52 weeks.

14. The method of claims 1 and 2 wherein the method further comprises administering at least one anti-hepatitis C agent before, concurrently or after administration of the Pegylated Type III Interferon or pharmaceutical formulation.

15. The method of claim 14 wherein the anti-hepatitis C agent is selected from the group consisting of polymerase and/or protease inhibitors, A3AR agonists, Toll-Like Receptor agonists, monoclonal antibodies, Botanicals, anti-phospholipids, immunomodulators, anti-inflammatory drugs, thiazolides, broad spectrum immune stimulators, inflammatory/fibrosis inhibitors, cyclophilin inhibitors, pancaspase inhibitors, HCV immune globulins, antivirals, anti-infectives, RNA inhibitors, glucosidase I inhibitors, IRES inhibitors, bezafibrates, nucleoside analogs, Type I Interferons and Type II Interferons.

16. The method of claim 15 wherein the polymerase and/or protease inhibitor is VCH-916 (Virochem), GS9190 (Gilead), GSK625433 (GlaxcoSmithKline), ITMN-191 (R-7227; InterMune), R7128 (Pharmasset/Roche), VCH-759 (Virochem), R1626 (Roche), TMC435350 (Medivir/Tibotec), SCH503034 (Boceprevir, Schering-Plough), A-831 (Arrow Therapeutics), valopicitabine (NM283, Idenix Pharmaceuticals) or VX950 (Telaprevir, Vertex).

17. The method of claim 15 wherein the A3AR agonist is CF102 (Can-Fite).

18. The method of claim 15 wherein the Toll-Like Receptor agonist is IMO-2125 (Idera Pharmaceuticals), Isatoribine (ANA971, Anadys Pharmaceuticals) or Actilon (CPG10101, Coley Pharmaceutical Group).

19. The method of claim 15 wherein the monoclonal antibody is AB68 (XTL bio).

20. The method of claim 15 wherein the Botanical is PYN17 (Phynova).

21. The method of claim 15 wherein the anti-phospholipid is Bavituximab (formerly Tarvacin; Peregrine).

22. The method of claim 15 wherein the immunomodulator is NOV-205 (Novelos Therapeutics), Oglufanide disodium (Implicit Bioscience) or thymalfasin (thymosin alpha 1; SciClone/Sigma-Tau).

23. The method of claim 15 wherein the anti-inflammatory drug is CTS-1027 (Conatus) or JBK-122 (Jenken Biosciences).

24. The method of claim 15 wherein the thiazolides is Alinia (nitazoxanide; Romark Laboratories).

25. The method of claim 15 wherein the broad spectrum immune stimulator is SCV-07 (SciClone).

26. The method of claim 15 wherein the inflammatory/fibrosis inhibitor is MitoQ (mitoquinone; Antipodean Pharmaceuticals).

27. The method of claim 15 wherein the cyclophilin inhibitor is DEBIO-025 (Debio Pharm Group).

28. The method of claim 15 wherein the pancaspase inhibitor is PF-03491390 (formerly IDN-6556; Pfizer Pharmaceuticals).

29. The method of claim 15 wherein the HCV immune globulin is Civacir (Nabi).

30. The method of claim 15 wherein the antiviral is Suvus (Methylene blue, formerly BIVN-104 (Virostat); Bioenvision).

31. The method of claim 15 wherein the glucosidase I inhibitor is MX-3253 (celgosivir; Migenix).

32. The method of claim 15 wherein the IRES inhibitor is VGX-410C (Mifepristone; VGX Pharmaceuticals).

33. The method of claim 15 wherein the bezafibrate is Hepaconda (Giaconda).

34. The method of claim 15 wherein the nucleoside analog is ribavirin (Roches's Copegus or Schering-Plough's Rebetol) or viramidine (taribavirin (ribavirin pro-drug); Valeant Pharmaceuticals).

35. The method of claim 34 wherein the ribavirin or viramidine is administered orally once or twice daily to the patient at a dose amount of about 800-1200 mg.

36. The method of claim 15 wherein the Type I Interferon is Interferon alpha or pegylated Interferon alpha.

37. The method of claim 36 wherein the Interferon alpha or pegylated Interferon alpha is PEGASYS (pegylated interferon-alpha-2a or peg-IFN-α-2a; Roche), PEG-INTRON (pegylated interferon-alpha-2b or peg-IFN-α-2b; Schering-Plough), Belerofon (Nautilus Biotech), oral interferon alpha (Amarillo Biosciences), BLX-883 (Locteron; Biolex Therapeutics/OctoPlus), Multiferon (Viragen), Albuferon (Human Genome Sciences), Consensus Interferon or (Infergen; Three Rivers Pharma).

38. The method of claim 15 wherein the Type I Interferon is omega interferon (Intarcia Therapeutics).

39. A method of treating a human patient having a relapsing genotype I chronic hepatitis C infection following prior treatment comprising administering to the human patient a therapeutically effective amount of a Pegylated Type III Interferon.

40. A method of treating a human patient having a relapsing genotype I chronic hepatitis C infection following prior treatment comprising administering to the human patient a therapeutically effective amount of a pharmaceutical formulation comprising a Pegylated Type III Interferon and a pharmaceutically acceptable vehicle.

41. The method of claims 39 and 40 wherein the dosing schedule is selected from the group consisting of one dose per week, two doses per week, three doses per week, one dose every other day, one dose every three days, and one dose every two weeks.

42. The method of claims 39 and 40 wherein the Type III Interferon is selected from the group consisting of an IL-28A polypeptide, an IL-28B polypeptide, and an IL-29 polypeptide.

43. The method of claim 42 wherein the IL-28A polypeptide is selected from the group consisting of SEQ ID NOs:2, 4, 6, 8, 10 and 12.

44. The method of claim 42 wherein the IL-28B polypeptide is selected from the group consisting of SEQ ID NOs:14, 16, 18, 20, 22, 24, 26, 28, 30 and 32.

45. The method of claim 42 wherein the IL-29 polypeptide is selected from the group consisting of SEQ ID NOs:34, 36, 38, 40, 42, 44, 46, 48, 50, 52, 54, 56, 58, 60, 62, 64, 66, 68, 70, 72, 74, 76, 78, 80, 82, 84, 86, 88, 90, 92, 94, 96, 98, 100, 102, 104, 106, 108, 110, 115, 117, 119, 121 and 123.

46. The method of claims 39 and 40 wherein the Pegylated Type III Interferon or pharmaceutical formulation is administered parenterally.

47. The method of claim 46 wherein the parenterally administered Pegylated Type III Interferon or pharmaceutical formulation is by injection or infusion.

48. The method of claim 46 wherein the Pegylated Type III Interferon or pharmaceutical formulation is administered intravenously, intramuscularly, subcutaneously, intradermally, or intraperitoneally.

49. The method of claims 39 and 40 wherein the therapeutically effective amount of Pegylated Type III Interferon or pharmaceutical formulation is administered to the patient in a dosing amount selected from the group consisting of less than 0.5 μg/kg, 0.5 to 1.0 μg/kg, 1.0 to 1.5 μg/kg, 1.5 to 2.0 μg/kg, 2.0 to 2.5 μg/kg, 2.5 to 3.0 μg/kg, 3.0 to 3.5 μg/kg, 3.5 to 4.0 μg/kg, 4.0 to 4.5 μg/kg, 4.5 to 5.0 μg/kg, 5.0 to 5.5 μg/kg, 5.5 to 6.0 μg/kg, 6.0 to 6.5 μg/kg, 6.5 to 7.0 μg/kg, 7.0 to 7.5 μg/kg, 7.5 to 8.0 μg/kg, 8.0 to 8.5 μg/kg, 8.5 to 9.0 μg/kg, 9.0 to 9.5 μg/kg, 9.5 to 10.0 μg/kg, greater than 10.0 μg/kg, fixed dose of about 60-80 μg, fixed dose of about 80-100 μg, fixed dose of about 100-120 μg, fixed dose of about 120-140 μg, fixed dose of about 140-160 μg, fixed dose of about 160-180 μg, fixed dose of about 180-200 μg, fixed dose of about 200-220 μg, fixed dose of about 220-240 μg, fixed dose of about 240-260 μg, fixed dose of about 260-280 μg, and fixed dose of about 280-300 μg.

50. The method of claims 39 and 40 wherein the duration of the treatment is less than 20 weeks, 20-24 weeks, 24-28 weeks, 28-32 weeks, 32-36 weeks, 36-40 weeks, 40-44 weeks, 44-48 weeks, 48-52 weeks, or greater than 52 weeks.

51. The method of claims 39 and 40 wherein the treatment further comprises at least one anti-hepatitis C agent.

52. The method of claim 51 wherein the anti-hepatitis C agent is selected from the group consisting of polymerase and/or protease inhibitors, A3AR agonists, Toll-Like Receptor agonists, monoclonal antibodies, Botanicals, anti-phospholipids, immunomodulators, anti-inflammatory drugs, thiazolides, broad spectrum immune stimulators, inflammatory/fibrosis inhibitors, cyclophilin inhibitors, pancaspase inhibitors, HCV immune globulins, antivirals, anti-infectives, RNA inhibitors, glucosidase I inhibitors, IRES inhibitors, bezafibrates, nucleoside analogs, Type I Interferons and Type II Interferons.

53. The method of claim 52 wherein the polymerase and/or protease inhibitor is VCH-916 (Virochem), GS9190 (Gilead), GSK625433 (GlaxcoSmithKline), ITMN-191 (R-7227; InterMune), R7128 (Pharmasset/Roche), VCH-759 (Virochem), R1626 (Roche), TMC435350 (Medivir/Tibotec), SCH503034 (Boceprevir, Schering-Plough), A-831 (Arrow Therapeutics), valopicitabine (NM283, Idenix Pharmaceuticals) or VX950 (Telaprevir, Vertex).

54. The method of claim 52 wherein the A3AR agonist is CF102 (Can-Fite).

55. The method of claim 52 wherein the Toll-Like Receptor agonist is IMO-2125 (Idera Pharmaceuticals), Isatoribine (ANA971, Anadys Pharmaceuticals) or Actilon (CPG10101, Coley Pharmaceutical Group).

56. The method of claim 52 wherein the monoclonal antibody is AB68 (XTL bio).

57. The method of claim 52 wherein the Botanical is PYN17 (Phynova).

58. The method of claim 52 wherein the anti-phospholipid is Bavituximab (formerly Tarvacin; Peregrine).

59. The method of claim 52 wherein the immunomodulator is NOV-205 (Novelos Therapeutics), Oglufanide disodium (Implicit Bioscience) or thymalfasin (thymosin alpha 1; SciClone/Sigma-Tau).

60. The method of claim 52 wherein the anti-inflammatory drug is CTS-1027 (Conatus) or JBK-122 (Jenken Biosciences).

61. The method of claim 52 wherein the thiazolides is Alinia (nitazoxanide; Romark Laboratories).

62. The method of claim 52 wherein the broad spectrum immune stimulator is SCV-07 (SciClone).

63. The method of claim 52 wherein the inflammatory/fibrosis inhibitor is MitoQ (mitoquinone; Antipodean Pharmaceuticals).

64. The method of claim 52 wherein the cyclophilin inhibitor is DEBIO-025 (Debio Pharm Group).

65. The method of claim 52 wherein the pancaspase inhibitor is PF-03491390 (formerly IDN-6556; Pfizer Pharmaceuticals).

66. The method of claim 52 wherein the HCV immune globulin is Civacir (Nabi).

67. The method of claim 52 wherein the antiviral is Suvus (Methylene blue, formerly BIVN-104 (Virostat); Bioenvision).

68. The method of claim 52 wherein the glucosidase I inhibitor is MX-3253 (celgosivir; Migenix).

69. The method of claim 52 wherein the IRES inhibitor is VGX-410C (Mifepristone; VGX Pharmaceuticals).

70. The method of claim 52 wherein the bezafibrate is Hepaconda (Giaconda).

71. The method of claim 52 wherein the nucleoside analog is ribavirin (Roches's Copegus or Schering-Plough's Rebetol) or viramidine (taribavirin (ribavirin pro-drug); Valeant Pharmaceuticals).

72. The method of claim 71 wherein the ribavirin or viramidine is administered orally once or twice daily to the patient at a dose of about 800-1200 mg.

73. The method of claim 52 wherein the Type I Interferon is Interferon alpha or pegylated Interferon alpha.

74. The method of claim 73 wherein the Interferon alpha or pegylated Interferon alpha is PEGASYS (pegylated interferon-alpha-2a or peg-IFN-α-2a; Roche), PEG-INTRON (pegylated interferon-alpha-2b or peg-IFN-α-2b; Schering-Plough), Belerofon (Nautilus Biotech), oral interferon alpha (Amarillo Biosciences), BLX-883 (Locteron; Biolex Therapeutics/OctoPlus), Multiferon (Viragen), Albuferon (Human Genome Sciences) or Consensus Interferon(Infergen; Three Rivers Pharma).

75. The method of claims 1, 2, 39 and 40 wherein the polyethylene glycol (PEG) of the Pegylated Type III Interferon is 20 kD or 30 kD mPEG-propionaldehyde.

76. A method of treating a human patient infected or at risk of infection with the hepatitis C virus comprising subcutaneously administering to the human patient about 1.5-5.0 μg/kg of a pegylated polypeptide, wherein the polypeptide comprises amino acid residues 1-176 of SEQ ID NO:106, and wherein the pegylated polypeptide is pegylated with mPEG propionaldehyde.

77. A method of treating a human patient infected or at risk of infection with the hepatitis C virus comprising subcutaneously administering to the human patient a pharmaceutical formulation comprising about 1.5-5.0 μg/kg of a pegylated polypeptide and a pharmaceutically acceptable vehicle, wherein the polypeptide comprises amino acid residues 1-176 of SEQ ID NO:106, wherein the pegylated polypeptide is pegylated with mPEG propionaldehyde.

78. The method of claims 76 and 77 wherein the mPEG propionaldehyde has a molecular weight of about 20 kD or 30 kD.

79. The method of claims 76 and 77 wherein the mPEG propionaldehyde is linear.

80. The method of claims 76 and 77 further comprising administering a nucleoside analog before, concurrently or after administration of the pegylated polypeptide or pharmaceutical formulation.

81. The method of claims 76 and 77 wherein the patient is selected from a subpopulation of hepatitis C patients consisting of treatment naïve patients with genotype I hepatitis C; treatment naïve patients with any genotype hepatitis C; patients co-infected with the human immunodeficiency virus (HIV); patients intolerant to Pegylated Interferon Alpha, Interferon Alpha or any other Pegylated or NonPegylated Type I Interferon; patients for whom treatment with Pegylated Interferon Alpha, Interferon Alpha or any other Pegylated or NonPegylated Type I Interferon is contraindicated; patients awaiting or following liver transplant; patients with decompensated liver disease; patients who are previous non-responders to treatment with Pegylated Interferon Alpha, Interferon Alpha or any other Pegylated or NonPegylated Type I Interferon either as a single agent or in combination with ribavirin or any other anti-hepatitis C agent, including patients who were null responders, responder/relapsers, or break-through patients; patients who were non-compliant with prior treatment with Pegylated Interferon Alpha, Interferon Alpha or any other Pegylated or NonPegylated Type I Interferon either as a single agent or in combination with ribavirin or other any of the anti-hepatitis C agents; patients with any base level of hepatitis C RNA; and patients with cirrhosis.

82. The method of claims 76 and 77 wherein the duration of the treatment is less than 20 weeks, 20 weeks, 24 weeks, 28 weeks, 32 weeks, 36 weeks, 40 weeks, 44 weeks, 8 weeks, 52 weeks or greater than 52 weeks.

83. A method of treating a responder/relapser human patient infected with the hepatitis C virus comprising subcutaneously administering to the human patient about 1.5-5.0 μg/kg of a pegylated polypeptide, wherein the polypeptide comprises amino acid residues 1-176 of SEQ ID NO:106, and wherein the pegylated polypeptide is pegylated with mPEG propionaldehyde having molecular weight of about 20 kD.

84. A method of treating a responder/relapser human patient infected with the hepatitis C virus comprising subcutaneously administering to the human patient a pharmaceutical formulation comprising about 1.5-5.0 μg/kg of a pegylated polypeptide and a pharmaceutically acceptable vehicle, wherein the polypeptide comprises amino acid residues 1-176 of SEQ ID NO:106, wherein the pegylated polypeptide is pegylated with mPEG propionaldehyde having a molecular weight of about 20 kD.

85. The method of claims 83 and 84 wherein the duration of the treatment is less than less than 20 weeks, 20-24 weeks, 24-28 weeks, 28-32 weeks, 32-36 weeks, 36-40 weeks, 40-44 weeks, 44-48 weeks, 48-52 weeks, or greater than 52 weeks.

86. A method of treating a treatment naïve human patient infected or at risk of infection with the hepatitis C virus comprising subcutaneously administering to the human patient about 1.5-5.0 μg/kg of a pegylated polypeptide, wherein the polypeptide comprises amino acid residues 1-176 of SEQ ID NO:106, and wherein the pegylated polypeptide is pegylated with mPEG propionaldehyde having molecular weight of about 20 kD.

87. A method of treating a treatment naïve human patient infected or at risk of infection with the hepatitis C virus comprising subcutaneously administering to the human patient a pharmaceutical formulation comprising about 1.5-5.0 μg/kg of a pegylated polypeptide and a pharmaceutically acceptable vehicle, wherein the polypeptide comprises amino acid residues 1-176 of SEQ ID NO:106, wherein the pegylated polypeptide is pegylated with mPEG propionaldehyde having a molecular weight of about 20 kD.

88. The method of claims 86 and 87 wherein the method further comprises administering a nucleoside analog to the patient.

89. The method of claim 88 wherein the nucleoside analog is ribavirin or viramidine.

90. The method of claim 89 wherein the ribavirin or viramidine is administered orally once or twice daily to the patient at a dose amount of about 800-1200 mg.
